SEQUENCE LISTING (95 sequences)

SEQ ID NO: 1 - DNA, 1629 bp, Thermotoga neapolitana
aaaaaatactcctacacaatgagtgaaggaaaccttccgattgcagagggaacgatcgcatatcttgagtacgaaaaagcgggcaggttcgaagcgggagatcacatcgtctacgttagaacggtgagaaggcaaaagcctgctgaaagacgaacaaccacttctgtttgaagaacacaggctgatatagaagcagggaacttccctgccttcatttttttcaacagttttgaaaacatcctgaacagaacgcatatacgatagagatcgggaggtgattcaggtgagaagactcatcgtgctggttctggctcttttgctggttgctttttcattttcacaggtgaagaacatcatctacttcatcggagacggaatgggactttcacaggcgtatctcacgagtctgatcgagggaagaccgctctcgttcatgaaaaccccttacacaggtcttgtgaagacccactctgtggacagctgggtgacggattctgccgccgcgggaactgctctggcaagcggttttaagacgaaaaacggtatgatcaacgttcttcccgacggcacaagggttcccaccatctttgagatagcaaaagcatatggagccaaaacgggcatcgttgtcacctgcagagtgacacatgcgacaccggcagccttctatgcgcatgtcaaaagcagaagcgaagaaaacgagatagcaaggcaactcgtggaaagtgaaacagtggatctggtcatgggaggaggatgggcaaactttcttcctgaaagccttggtggtaaaagatcggacggtctcaatctcatcgaaatggcgaagaaaaagggctacgtctatgtgacaaaaaaggaagatctgatgaaacttccagagaacaccgaaaaggtcctggcgctttttgcgccgagccatcttgacccagcaagcagtagagaggaacagcccatgctctacgaaatggtgaagaaggccctggaaatactctcaaatgatgaagagcccttcttcctcatggtggaaggttctcagatcgattgggaagcacacgacaacgatatctacggtgtttggaaagaagtcgtggagttcgacagagcggttcaggtggctctcgactttgccctcaaaagaggagataccctcgtgatcgtgacagcagaccatgaaacaggaggtcttgcactctcttcgggtgactacagggttgatgtggacagaatcagaaagttctcaaagaccaccgactggatcttggcgaattactctttgaaagatcgcgaaagtttcaaaaaggccatcgaaaaatacttcggtctgactctgtccgactcggatctcgacagaatcacctcttcaaacaattccaaagtagaactcggaagggtgttgagcgaaaaggtgaacgttggctggaccaccacatcacactccggtgttccggtccccatctatgcattcggtcccggtgcggagaacttcacaggatttctggacaacaccgatatcccgaggatcatgatgaaactcgctggatactcccttcagtatccactcgtgaaagaacctgtgaaatgaaatgaggggctctgtgccctcattcaaacacgtccgattcttcg

SEQ ID NO: 2 - protein, 433 aa, Thermotoga neapolitana
MRRLIVLVLALLLVAFSFSQVKNIIYFIGDGMGLSQAYLTSLIEGRPLSFMKTPYTGLVKTHSVDSWVTDSAAAGTALASGFKTKNGMINVLPDGTRVPTIFEIAKAYGAKTGIVVTCRVTHATPAAFYAHVKSRSEENEIARQLVESETVDLVMGGGWANFLPESLGGKRSDGLNLIEMAKKKGYVYVTKKEDLMKLPENTEKVLALFAPSHLDPASSREEQPMLYEMVKKALEILSNDEEPFFLMVEGSQIDWEAHDNDIYGVWKEVVEFDRAVQVALDFALKRGDTLVIVTADHETGGLALSSGDYRVDVDRIRKFSKTTDWILANYSLKDRESFKKAIEKYFGLTLSDSDLDRITSSNNSKVELGRVLSEKVNVGWTTTSHSGVPVPIYAFGPGAENFTGFLDNTDIPRIMMKLAGYSLQYPLVKEPVK

SEQ ID NO: 3 - DNA, 1269 bp, Thermotoga neapolitana
atggctagccaggtgaagaacatcatctacttcatcggagacggaatgggactttcacaggcgtatctcccgcgtctgatcgagggaagaccgctctcgttcatgaaaaccccttacacaggtcttgtgaagaaccactctgtggacagctgggtgacggattctgccgccgcgggaactgctctggcaagcggtttaagacgaaaacggtatgatcaacgttcttcccgacggcacaaggttcccaccatctttgagatagcaaaagcatatggagccaaaacgggcatcgttgtcacctgcagagtgacacatgcgacaccggcagccttgtatgcgcatgtcaaaagcagaagcgaagaaaacgagatagcaaggcaactcgtggaaagtgaaacagtggatctggtcatgggaggaggatgggcaaactttcttcctgaaagccttggtggtaaaacatcggacggtctcaatctcatcgaaatggcgaacaaaaagggctacgtctatgtgacaaaaaaggaagatctgatgaaacttccagagaacaccgaaaaggtcctggcgctttttgcgccgagccatcttgacccagcaagcagtagagaggaacagcccatgctctacgaaatggtgaagaaggccctggaaatactctcaaatgatgaagagcccttcttcctcatggtggaaggttctcagatcgactgggaagcacacgacaacgatatctacggtgtttggaaagaagtcgtggagttcgacagagcggttcaggtggctctcgactttgccctcaaaagaggagataccctcgtgacagcagaccatgaaacaggaggtcttgctctctcttcgggtgactacagggttgatgtggacagaatcagaaagttctcaaagaccaccgactggatcttggcgaattactctttgaaagatcgcgaaagtttcaaaaaggccatcgaaaaatacttcggtctgactctgtccgactcggatctcgacagaatcacctcttcaaacaattccaaagtagaactcggaagggtgttgagcgaaaaggtgaacgttggctggaccaccacatcacactccggtgttccggtccccatctatgcattcggtcccggtgcggagaacttcacaggatttctggacaacaccgatatcccgaggatcatgatgaaactcgctggatactcccttcagtatccactcgtgaaagaacctgtgaaactcgagcaccaccaccaccaccactga

SEQ ID NO: 4 - protein, 422 aa, Thermotoga neapolitana
MASQVKNIIYFIGDGMGLSQAYLPRLIEGRPLSFMKTPYTGLVKNHSVDSWVTDSAAAGTALASGLRRKRYDQRSSRRHKVPTIFEIAKAYGAKTGIVVTCRVTHATPAALYAHVKSRSEENEIARQLVESETVDLVMGGGWANFLPESLGGKTSDGLNLIEMANKKGYVYVTKKEDLMKLPENTEKVLALFAPSHLDPASSREEQPMLYEMVKKALEILSNDEEPFFLMVEGSQIDWEAHDNDIYGVWKEVVEFDRAVQVALDFALKRGDTLVTADHETGGLALSSGDYRVDVDRIRKFSKTTDWILANYSLKDRESFKKAIEKYFGLTLSDSDLDRITSSNNSKVELGRVLSEKVNVGWTTTSHSGVPVPIYAFGPGAENFTGFLDNTDIPRIMMKLAGYSLQYPLVKEPVKLEHHHHHH

SEQ ID NO: 5 - DNA, 1242 bp, Thermotoga neapolitana
atggctagccaggtgaagaacatcatctacttcatcggagacggaatgggactttcacaggcgtatctcccgcgtctgatcgagggaagaccgctctcgttcatgaaaaccccttacacaggtcttgtgaagaaccactctgtggacagctgggtgacggattctgccgccgcgggaactgctctggcaagcggtttaagacgaaaacggtatgatcaacgttcttcccgacggcacaaggttcccaccatctttgagatagcaaaagcatatggagccaaaacgggcatcgttgtcacctgcagagtgacacatgcgacaccggcagccttgtatgcgcatgtcaaaagcagaagcgaagaaaacgagatagcaaggcaactcgtggaaagtgaaacagtggatctggtcatgggaggaggatgggcaaactttcttcctgaaagccttggtggtaaaacatcggacggtctcaatctcatcgaaatggcgaacaaaaagggctacgtctatgtgacaaaaaaggaagatctgatgaaacttccagagaacaccgaaaaggtcctggcgctttttgcgccgagccatcttgacccagcaagcagtagagaggaacagcccatgctctacgaaatggtgaagaaggccctggaaatactctcaaatgatgaagagcccttcttcctcatggtggaaggttctcagatcgactgggaagcacacgacaacgatatctacggtgtttggaaagaagtcgtggagttcgacagagcggttcaggtggctctcgactttgccctcaaaagaggagataccctcgtgacagcagaccatgaaacaggaggtcttgctctctcttcgggtgactacagggttgatgtggacagaatcagaaagttctcaaagaccaccgactggatcttggcgaattactctttgaaagatcgcgaaagtttcaaaaaggccatcgaaaaatacttcggtctgactctgtccgactcggatctcgacagaatcacctcttcaaacaattccaaagtagaactcggaagggtgttgagcgaaaaggtgaacgttggctggaccaccacatcacactccggtgttccggtccccatctatgcattcggtcccggtgcggagaacttcacaggatttctggacaacaccgatatcccgaggatcatgatgaaactcgctggatactcccttcagtatccactcgtgaaagaacctgtgaaa

SEQ ID NO: 6 - protein, 414 aa, Thermotoga neapolitana
MASQVKNIIYFIGDGMGLSQAYLPRLIEGRPLSFMKTPYTGLVKNHSVDSWVTDSAAAGTALASGLRRKRYDQRSSRRHKVPTIFEIAKAYGAKTGIVVTCRVTHATPAALYAHVKSRSEENEIARQLVESETVDLVMGGGWANFLPESLGGKTSDGLNLIEMANKKGYVYVTKKEDLMKLPENTEKVLALFAPSHLDPASSREEQPMLYEMVKKALEILSNDEEPFFLMVEGSQIDWEAHDNDIYGVWKEVVEFDRAVQVALDFALKRGDTLVTADHETGGLALSSGDYRVDVDRIRKFSKTTDWILANYSLKDRESFKKAIEKYFGLTLSDSDLDRITSSNNSKVELGRVLSEKVNVGWTTTSHSGVPVPIYAFGPGAENFTGFLDNTDIPRIMMKLAGYSLQYPLVKEPVK

SEQ ID NO: 7 - DNA, 1290 bp, Thermotoga neapolitana
gtgagaagactcatcgtgctggttctggctcttttgctggttgctttttcattttcacaggtgaagaacatcatctacttcatcggagacggaatgggactttcacaggcgtatctcccgcgtctgatcgagggaagaccgctctcgttcatgaaaaccccttacacaggtcttgtgaagaaccactctgtggacagctgggtgacggattctgccgccgcgggaactgctctggcaagcggtttaagacgaaaacggtatgatcaacgttcttcccgacggcacaaggttcccaccatctttgagatagcaaaagcatatggagccaaaacgggcatcgttgtcacctgcagagtgacacatgcgacaccggcagccttgtatgcgcatgtcaaaagcagaagcgaagaaaacgagatagcaaggcaactcgtggaaagtgaaacagtggatctggtcatgggaggaggatgggcaaactttcttcctgaaagccttggtggtaaaacatcggacggtctcaatctcatcgaaatggcgaacaaaaagggctacgtctatgtgacaaaaaaggaagatctgatgaaacttccagagaacaccgaaaaggtcctggcgctttttgcgccgagccatcttgacccagcaagcagtagagaggaacagcccatgctctacgaaatggtgaagaaggccctggaaatactctcaaatgatgaagagcccttcttcctcatggtggaaggttctcagatcgactgggaagcacacgacaacgatatctacggtgtttggaaagaagtcgtggagttcgacagagcggttcaggtggctctcgactttgccctcaaaagaggagataccctcgtgacagcagaccatgaaacaggaggtcttgctctctcttcgggtgactacagggttgatgtggacagaatcagaaagttctcaaagaccaccgactggatcttggcgaattactctttgaaagatcgcgaaagtttcaaaaaggccatcgaaaaatacttcggtctgactctgtccgactcggatctcgacagaatcacctcttcaaacaattccaaagtagaactcggaagggtgttgagcgaaaaggtgaacgttggctggaccaccacatcacactccggtgttccggtccccatctatgcattcggtcccggtgcggagaacttcacaggatttctggacaacaccgatatcccgaggatcatgatgaaactcgctggatactcccttcagtatccactcgtgaaagaacctgtgaaa

SEQ ID NO: 8 - protein, 411 aa, Thermotoga neapolitana
QVKNIIYFIGDGMGLSQAYLPRLIEGRPLSFMKTPYTGLVKNHSVDSWVTDSAAAGTALASGLRRKRYDQRSSRRHKVPTIFEIAKAYGAKTGIVVTCRVTHATPAALYAHVKSRSEENEIARQLVESETVDLVMGGGWANFLPESLGGKTSDGLNLIEMANKKGYVYVTKKEDLMKLPENTEKVLALFAPSHLDPASSREEQPMLYEMVKKALEILSNDEEPFFLMVEGSQIDWEAHDNDIYGVWKEVVEFDRAVQVALDFALKRGDTLVTADHETGGLALSSGDYRVDVDRIRKFSKTTDWILANYSLKDRESFKKAIEKYFGLTLSDSDLDRITSSNNSKVELGRVLSEKVNVGWTTTSHSGVPVPIYAFGPGAENFTGFLDNTDIPRIMMKLAGYSLQYPLVKEPVK

SEQ ID NO: 9 - protein, 19 aa, Thermotoga neapolitana
MRRLIVLVLALLLVAFSFS

SEQ ID NO: 10 - DNA, 57 bp, Thermotoga neapolitana
gtgagaagactcatcgtgctggttctggctcttttgctggttgctttttcattttca

SEQ ID NO: 11 - DNA, 93 bp, Thermotoga neapolitana
gtgaacgttggctggaccaccacatcacactccggtgttccggtccccatctatgcattcggtcccggtgcggagaacttcacaggatttctg

SEQ ID NO: 12 - DNA, 862 bp, Thermotoga neapolitana
ctgcagagtgacacatgcgacaccggcagccttgtatgcgcatgtcaaaagcagaagcgaagaaaacgagatagcaaggcaactcgtggaaagtgaaacagtggatctggtcatgggaggaggatgggcaaactttcttcctgaaagccttggtggtaaaacatcggacggtctcaatctcatcgaaatggcgaacaaaaagggctacgtctatgtgacaaaaaaggaagatctgatgaaacttccagagaacaccgaaaaggtcctggcgctttttgcgccgagccatcttgacccagcaagcagtagagaggaacagcccatgctctacgaaatggtgaagaaggccctggaaatactctcaaatgatgaagagcccttcttcctcatggtggaaggttctcagatcgactgggaagcacacgacaacgatatctacggtgtttggaaagaagtcgtggagttcgacagagcggttcaggtggctctcgactttgccctcaaaagaggagataccctcgtgacagcagaccatgaaacaggaggtcttgctctctcttcgggtgactacagggttgatgtggacagaatcagaaagttctcaaagaccaccgactggatcttggcgaattactctttgaaagatcgcgaaagtttcaaaaaggccatcgaaaaatacttcggtctgactctgtccgactcggatctcgacagaatcacctcttcaaacaattccaaagtagaactcggaagggtgttgagcgaaaaggtgaacgttggctggaccaccacatcacactccggtgttccggtccccatctatgcattcggtcccggtgcggagaacttcacaggatttctg

SEQ ID NO: 13 - protein, 450 aa, Escherichia coli
RTPEMPVLENRAAQGDITAPGGARRLTGDQTAALRDSLSDKPAKNIILLIGDGMGDSEITAARNYAEGAGGFFKGIDALPLTGQYTHYALNKKTGKPDYVTDSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLK

SEQ ID NO: 14 - protein, 430 aa, Bacillus subtilis
QEKKKGNQDEIKNVIVLIGDGMGVSYTSAYRYLKDNKKTKVVEPTAFDQYLVGQQTTYPDDPEQNVTDSAAAATAMSAGIKTYNNAIAVDNDGSEAKTVLEAAKEKGKATGLVATSEITHATPASFGSHDHSRKNMNSIADDYFDEMVNGKHKIDVLLGGGKSNFDRKDRNLIKEFKKAGYSYVDDRKDMLKNKDSQVLGLFADGGLPKKIDRTKDIPSLKDMTNTAIKKLNKDKDGFFLMVEGSQIDWAGHDNDIVGAMSEMEDFEQAYKAAIDFAKKDKHTLVVATADHSTGGYSIGADSIYNWFSEPIKAAKRTPDFMAEKIADGADVEKTLKTYIDQKKLALTKAEIQSVEEAAKSKEVLDIDNAIENIFNKRSHTGWTTGGHTGEDVPVYAYGPSSETFAGQIDNTEIAKNVFKALQYNIKINDK

SEQ ID NO: 15 - protein, 480 aa, Bos taurus
LVPVEEEDPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCKTTRGNEVTSVMNRAKKAGKSVGVVTTTRVQHASPAGAYAHTVNRNWYSDADLPADAQMNGCQDIAAQLVNNMDIDVILGGGRKYMFPVGTPDPEYPDDASVNGVRKRKQNLVQAWQAKHQGAQYVWNRTALLQAADDSSVTHLMGLFEPADMKYNVQQDHTKDPTLQEMTEVALRVVSRNPRGFYLFVEGGRIDHGHHDDKAYMALTEAGMFDNAIAKANELTSELDTLILVTADHSHVFSFGGYTLRGTSIFGLAPSKALDSKSYTSILYGNGPGYALGGGSRPDVNDSTSEDPSYQQQAAVPQASETHGGEDVAVFARGPQAHLVHGVEEETFVAHIMAFAGCVEPYTDCNLPAPT

SEQ ID NO: 16 - protein, 37 aa, Thermotoga neapolitana
VNVGWTTTSHSGVPVPIYAFGPGAENFTGFLDNTDIP

SEQ ID NO: 17 - protein, 37 aa, Bacillus subtilis
SHTGWTTGGHTGEDVPVYAYGPSSETFAGQIDNTEIA

SEQ ID NO: 18 - protein, 37 aa, Thermotoga naphthophila
VSVGWTTTTHSGTPVPIFAFGPGAENFTGFLDNTEIP

SEQ ID NO: 19 - protein, 37 aa, Thermotoga maritima; identical to SEQ ID NO: 18

SEQ ID NO: 20 - protein, 35 aa, Oceanobacillus iheyensis
IGWTSTGHTAADIPIYAYGPSAESFSGKLDNTDIP

SEQ ID NO: 21 - protein, 32 aa, Geobacillus kaustophilus
WTTTGHTAADVPLTAMGPGAERFTGIYENTRI

SEQ ID NO: 22 - protein, 28 aa, Deinococcus geothermalis
HSADPVPLFAFGPGGNLFRNQIDQTEVT

SEQ ID NO: 23 - protein, 31 aa, Halobacterium sp.
IAFTTDGHTGTDVPVFAHGPNAARFDAARDN

SEQ ID NO: 24 - protein, 31 aa, Pseudomonas aeruginosa
SSQEHTGTQLRIAAYGPQAANVTGLTDQTDL

SEQ ID NO: 25 - protein, 36 aa, Flavobacterium sp.
VAVYFITDQHSGELIPVFAAGKGADHFKGIYQNSAI

SEQ ID NO: 26 - protein, 32 aa, Antarctic bacterium TAB5
FSTGGHSATLIPVFAYGPGSEEFIGIYENNEI

SEQ ID NO: 27 - protein, 6 aa, Artificial Sequence
  Synthetic
VNVGWT

SEQ ID NO: 28 - protein, 7 aa, Artificial Sequence
  Synthetic
ENFTGFL

SEQ ID NO: 29 - skipped

SEQ ID NO: 30 - DNA, 20 bp, Artificial Sequence
  Synthetic
  misc_feature (3)..(3): n is a, c, g, or t
  misc_feature (9)..(9): n is a, c, g, or t
  misc_feature (12)..(12): n is a, c, g, or t
  misc_feature (18)..(18): n is a, c, g, or t
gtnaaygtnggntggacnac

SEQ ID NO: 31 - DNA, 24 bp, Artificial Sequence
  Synthetic
  misc_feature (2)..(2): n is a, c, g, or t
  misc_feature (8)..(8): n is a, c, g, or t
  misc_feature (11)..(11): n is a, c, g, or t
  misc_feature (23)..(23): n is a, c, g, or t
cnarraanccngtraarttytcng

SEQ ID NO: 32 - DNA, 20 bp, Artificial Sequence
  Synthetic
gaccagatccactgtttcac

SEQ ID NO: 33 - DNA, 19 bp, Artificial Sequence
  Synthetic
cacgagttgccttgctatc

SEQ ID NO: 34 - DNA, 30 bp, Artificial Sequence
  Synthetic
tatctcgagtttcacaggttctttcacgag

SEQ ID NO: 35 - DNA, 30 bp, Artificial Sequence
  Synthetic
atagctagccaggtgaagaacatcatctac

SEQ ID NO: 36-37 - skipped (2 entries)

SEQ ID NO: 38 - protein, 5 aa, Thermotoga neapolitana
QVKNI

SEQ ID NO: 39 - DNA, 1629 bp, Thermotoga neapolitana; identical to SEQ ID NO: 1

SEQ ID NO: 40 - DNA, 1305 bp, Thermotoga maritima
atgaaaaggctttttacactgatactgatttccctgctggtcgcgctcgctttttctcaggtgaagaacgttatctacctcattggagacggtatgggtctttctcaagtctatctcaccagcatgctcgaaggaagacccctctcgttcatgaagactccttacatcggacttgtgaagacacactccgcaaacagctgggtgactgattccgcagcagcgggcacagcgctggcatcggggttcaaaacgaacaacggtatgatcaacattctccccgatggaacggtggtacccactatcttcgaagtggcaaagacctacggagttcgaacgggtatcgttgttacctgcagagtaacacatgcgacgccggctgctttctacgcacacgttaaaagtagagatgaagagaacgaaatagcaagacaactcgttgagaatgaaacagtcgatgtggtgatggggggaggttgggcaaactttcttcccaaggatctcggtggaaaacgagatgacaacctcaacttgattgaacttgcaaaagaaaaagggtatatatacgtgaaaaccagagaggaactctcaaaaatctccgcggattcagataaaatactcgctctctttgcaccaagtcacctggatcctgcaagtagcagaaaggagcagccgatgctctatgaaatggtggaaaaagcgctcgagatactgtcgaaagatgatgaacccttcttcctcatggtggaaggctcccagatagactgggaagcccacgataacgacatctacggggtctggaaagaagtcgtggaattcgacaaagccgttcaggtggcgctcgatttcgccctcaagaggggagacaccctcgtcatcgtaactgccgaccacgaaacgggtggattagggctttcttcgggcgactacagagtagatgtggacaaaatcagaaacttcaaaaaaacaacggactggatcatggcaaattactcgccaaaagatcgggagaaattcaaaaaggccatcgaagagtatttcggtttgactctttctgatgaggatctgaacaggatttccatgtccaaaaatccaaagatcgaacttggaaggatcctgggagaaaaggttagcgttgggtggaccaccacaacacattcaggaactcccgttcccatcttcgcttttggtccgggtgccgaaaacttcaccggatttctcgacaacaccgagattccaaggatcataatgaaactcacagggtactctcttcagtatccacttctgaaagaacccgtaacgaaatga

SEQ ID NO: 41 - DNA, 1305 bp, Thermotoga naphthophila
atgaaaaggctttttacgctgatactgatttccctgctggtcgcgctcgttttttctcaggtgaaaaacgttatctatttcattggagatggtatgggtctttctcaagtctatctcaccagcatgctcgaaggaagaccactctcgctcatgaagactccttacatcggacttgtgaagacacactccgcaaacagctgggtgactgattccgcagcagcgggcacagcgctggcatcggggttcaaaacgaacaacggtatgatcaacattctccccgatggaacggtggtacccactatcttcgaagtggcaaagacctacggagttcgaacgggtatcgttgttacctgcagagtaacacatgcgacgccggctgctttctacgcacacgttaaaagtagagatgaagagaacgaaatagcaagacaactcgttgagaatgaaacagtcgatgtggtgatggggggaggttgggcaaactttcttcccaaggatctcggtggaaaacgagatgacaacctcaacttgattgaacttgcaaaagaaaaagggtatatatacgtgaaaaccagagaggaactctcaaaaatctccgcggattcagataaaatactcgctctctttgcaccaagtcacctggatcctgcaagtagcagaaaggagcagccgatgctctatgaaatggtggaaaaagtgctcgagatactgtcgaaagatgatgaacccttcttcctcatggtggaaggctcccagatagactgggaagcccacgataacgacatctacggggtctggaaagaagtcaaggaattcgacaaagccgttcaggtggcgcttgatttcgccctcaagaggggagacaccctcgtcattgtaactgccgaccacgaaacgggtggattagggctttcttcgggcgactacagagtagatgtggacaaaatcagaaacttcaaaaaaacaacggactggatcatggcaaattactctccaaaagatcaagagaaattcaaaaaggccatcgaagagtatttcggtttgactctttctgatgaggatctgaacaggatttccatgtccaaaaatccaaagatcgaacttggaaggatcctgggagaaaaggttagcgttgggtggaccaccacaacacattcaggaactcccgttcccatcttcgcttttggtccgggtgccgaaaacttcaccggatttctcgacaacaccgagattccaaggatcataatgaaactcacagggtactctcttcaatatccacttctgaaagaacccgtaacgaaatga

SEQ ID NO: 42 - DNA, 1335 bp, Bacillus halodurans
ttagctcgctttttcccactcaagatccatcacatctgcgaggatgagtggtatgtctgtgttgtctaacaatccatgaagccgatcagagcaaggcccgaacgcataaataggaatatccgttcccgtgtgaacaggggtcgtccatcctatcaacgcacgtctgctaatgacttgattaatggcattcgttaagtgatccgcctttttaatttgtttgagctctttttttgttagctcaaacccagtgtatgttttcataacgtcagctaggttggtccggcgtttgttcacttgactggcaataaatttcccagttgcttttacttgtttgagcatatcgggttttgctttgtactgtccatatgcacctacggaaagtccacccgtatcatggtcgcctacgacaacgacgagggtattcccgtccttctccgcaaaatcaaacgcgacgctcaccgcttcttcaaacgcttcaatatccttcatggcccacgcagcatcattgtcatgaccagcccaatcaatttggcttccttcaaccattaagaagaagccgttcggattttgttttaacgtgtggagcgcagcttctgtcatttctgctaagctcggctcttgtgtcccttgtcgatccagctctggtgccaaggctccctcggaaaataggccgagaacctttgctgaatttacattttgcagttcttcctttgtttcgatcaacgcataacccgcgtcctccgcttcatttagtagatgccggctattttgcattcctccttctgcttcgggaacaaacatttttttcccgccccctagtaacacatccacctgtccaatcatttgcggcgcaatcttcgcttcatcgattcttaatggaacacttgctacgaaggcggctggtgtcgcatgggtaagtcgagtcgtaacgacgattcctgtcccttttttctgctttcccgcagcttgtaaaatggactcaagctcttctccttcagtggacataccgatcgtcccgttgctcgtttttgtacctgtcgccagcgcagttccggcggcagccgaatcggtgacccaactgtcagccgaatgagtttttaccatcccgacaagatgggggtcccagatcggctcctcctcgcctttataaatacgataattggtagcataactagccgagtacccatcggggatcatataaataatgtttttaacttgttcgctgtcaggtgtgattgttccctcctgaacaaaagaagcaaacacggtttccgctggcaagataagcacaatcataacaaaaagtaagacggttacatgtacgcgatgtttcat

SEQ ID NO: 43 - DNA, 1416 bp, Escherichia coli
gtgaaacaaagcactattgcactggcactcttaccgttactgtttacccctgtgacaaaagcccggacaccagaaatgcctgttctggaaaaccgggctgctcagggcgatattactgcacccggcggtgctcgccgtttaacgggtgatcagactgccgctctgcgtgattctcttagcgataaacctgcaaaaaatattattttgctgattggcgatgggatgggggactcggaaattactgccgcacgtaattatgccgaaggtgcgggcggcttttttaaaggtatagatgccttaccgcttaccgggcaatacactcactatgcgctgaataaaaaaaccggcaaaccggactacgtcaccgactcggctgcatcagcaaccgcctggtcaaccggtgtcaaaacctataacggcgcgctgggcgtcgatattcacgaaaaagatcacccaacgattctggaaatggcaaaagccgcaggtctggcgaccggtaacgtttctaccgcagagttgcaggatgccacgcccgctgcgctggtggcacatgtgacctcgcgcaaatgctacggtccgagcgcgaccagtgaaaaatgtccgggtaacgctctggaaaaaggcggaaaaggatcgattaccgaacagctgcttaacgctcgtgccgacgttacgcttggcggcggcgcaaaaacctttgctgaaacggcaaccgctggtgaatggcagggaaaaacgctgcgtgaacaggcacaggcgcgtggttatcagttggtgagcgatgctgcctcactgaattcggtgacggaagcgaatcagcaaaaacccctgcttggcctgtttgctgacggcaatatgccagtgcgctggctaggaccgaaagcaacgtaccatggcaatatcgataagcccgcagtcacctgtacgccaaatccgcaacgtaatgacagtgtaccaaccctggcgcagatgaccgacaaagccattgaattgttgagtaaaaatgagaaaggctttttcctgcaagttgaaggtgcgtcaatcgataaacaggatcatgctgcgaatccttgtgggcaaattggcgagacggtcgatctcgatgaagccgtacaacgggcgctggaattcgctaaaaaggagggtaacacgctggtcatagtcaccgctgatcacgcccacgccagccagattgttgcgccggataccaaagctccgggcctcacccaggcgctaaataccaaagatggcgcagtgatggtgatgagttacgggaactccgaagaggattcacaagaacataccggcagtcagttgcgtattgcggcgtatggcccgcatgccgccaatgttgttggactgaccgaccagaccgatctcttctacaccatgaaagccgctctggggctgaaataa

SEQ ID NO: 44 - DNA, 1317 bp, Escherichia coli
gctcagagcgatattactgcacccggcggtgctcgccgcttaacgggtgatcagaccgccgctctgcgtgattctcttagcgataaacctgcaaaaaatattattttgctgattggcgatgggatgggggactcggaaattactgccgcacgcaattatgccgaaggtgcgggcggcttttttaaaggtatcgatgccttaccgcttaccgggcaatacactcactatgcgctgaataaaaaaaccggcaaaccggactacgtcaccgactcggctgcatcagcaaccgcctggtcaactggtgtcaaaacctataacggcgcgctgggcgtcgatattcacgaaaaagatcacccaacgattctggaaatggcaaaagccgcaggtctggcgaccggtaacgtttctaccgcagagttgcaggatgccacgcccgctgcgctggtggcgcatgtgacctcgcgcaaatgctacggtccgagtgcgaccagtgaaaaatgtccgggtaacgctctggaaaaaggcggaaaaggatcgattaccgaacagctgcttaacgcccgtgccgatgttacgcttggcggcggcgcaaaaacctttgctgaaacggcaaccgccggtgaatggcagggaaaaacgctgcgtgaacaggcacaggcgcgtggttatcagttggtgagcgatgctgcctcactgaattcggtgacggaagcgaatcagcaaaaacccctattaggactgtttgctgacggcaatatgccagtgcgctggcaaggaccgaaagcaacgtaccacggtaatatagataagcccgcagtcacctgtacgcctaatccgcaacgtaatgacagtgtaccgaccctggcgcagatgaccgacaaagccattgaattgttgagtaaaaatgagaaaggctttttcctgcaagttgaaggtgcgtcaatcgataaacaggatcacgctgcgaatccttgtggggcaattggcgagacggtcgatctcgatgaagccgtacaacgggcgctggaattcgctaaaaaggatggtaacacgttggtcatagtcaccgctgatcacgcccatgccagccagatagttgcgccagataccaaagctccgggcctcacccaggcgctaaataccaaagatggcgcagtgatggtgatgagttacgggaactccgaagaggattcacaagaacataccggcagtcagttgcgtattgcggcgtatggcccgcatgccgccaatgttgttggactgaccgaccagaccgatctcttctacaccatgaaagccgccctggggctgaaataa

SEQ ID NO: 45 - DNA, 1341 bp, Bacillus clausii
ttaaatccccagcgctgctttcatgattttcgggacttcagtgttgtcgatcgtgccggtaaaccgttctgccccggcgccgtaagcgtacagtggcacgtcaatgcctgtatgatctaacgatgaccaggctacgcctgctttttcgctgatggccgtattgatcgctaattccagtgagtcgctttctttcagttgatccaattcttcttctgtccattcaaaattggtgtacgtttcaagcacttcttttaagttgctataattccgttcgacttctgcagccatgtcgctccctagagcagagacaccatgaagcttagacggatccccttcttctgatgcgtgaacggccatcccccctgtatcatgatcgccgaccatgacgacaagcgtctcgccattctcatcggcaaaatcgaccgctgcttcgactgcttcttcaaaagcggccgtatctgtcatggcgtaataggcgtcattggcgtgtccagcccagtcaatttggctgccttctaccattaagaaaaacccattgtcctcttttgacaagaattcaattgctttttcagtcatctcccctaaactaggctcgtccgtccgtttattagctgccggcgacagtgcttcttcagcaaacaagccaagcaagcggtcttcttcaccattccagttcgctaattgccgcgctgtctctacaaatgcaaaccctttttcttctgcttcgcttattaaatcgacgccatcttcgcgaataccgccttggtcttcaggcacaaactctgtccggccaccaccaagcattaaatcgatatgatcgtttgcgaccatttgtgaggcgatttctgtataactgttccgtgactctacgctaacagcaaaagcggcaggcgtcgcgtgtgtaatcgttgatgtagccacaagtcctgtgcgcttgccgttttctttcgctgagtcgagaattgtcggcaatgtttgtccagcaggagttacgccgatcatgccattattcgttttttccccggttgctaaagctgtgcctgctgcagcggaatcggtcacttctgcatttgccgagtgcgtttgcacgaaggcgcgcagcatgttgcgttcgtcccaaataggctcgccatcttccttgtataaacggtaattgtttgtatagccttgcgaaaagccatcaggaattaaaaagatgacatttttggcttgtccactgctaggctgattgccttcttctcctacttccccactgtctgatgcaaatgccgccgtgtaggtcgctccaagacacacgatcccaaccagccctgccactgccaatttctttgaacgtttcat

SEQ ID NO: 46 - DNA, 1332 bp, Oceanobacillus iheyensis
gtgttaaagaaggtaggatggatttcagtaataagtctattaatctttgcaggaattatgggagcatcttttggtaacgaagaagatgcaacatcagcaaagggtaataataaaaaagtagagaatgtaatatatatgattccagatggatttagttcagattatgcttctaattaccgtgcctataaaggagaagaagtagtatgggattctcacttaaaaggtctttttacaacacattcagcagattcagatgttactgattcggcagcagccggcacagcaatggctacgggtgagaaaacaaacaatggtgttattggtaaagatacgaatgggaataatttagaaactattttagaaaaatccaaacaacatcataaagcaaccggattagtagctacatcaacaattacacacgcaactccagctgcatttgcaacccatgtagaagatagaaataatgaaacggagattgcacgacaattaattaaatctgaagtggatgttttacttggtggaggaatgaacaaatttattggcgaagaagaaggcggtaatcaagaagatggtgagctaatgcaaaaggctcaagatacgggctatatcttagctgaaaataaacaggaaatgctagaacaaaatatagatatcaagaaagacgaaaaattacttggcttgtttgcagatgaagcgttatctccagaacttcatagaaaagatacggaagaaccaagtttagaggacatgactacacatgcgattgatcaactgaatcagaataaaaaaggatttttcttaatggtggaaggtagtcaaattgattgggctggtcatgataatgattctgcctatgcgatgagtgatactgaagcatttgaaaaagcagttcaagctgcaattgattttgctgaagaggatggagaaacattagttgtagttgcggctgatcatgatacaggaggaatgacaactggtggatatgaccagatggaattaaatgcaaacattcttaatgatgtaacagcaactggagaatatatggctaatcaactagatgaagagcgttctaatgtccatgaagtcgtaaatacgtatacaggatttaaattgacagaagaagaaatagaatctattgaaacagcagaagatccaaaacttgctatcaatcatgtcgttagtgcacgatctaccattggttggacaagtactggtcacactgctgcagatattccgatttatgcatatggtccaagtgcagaatcattttctggcaaattggataacactgatattccaaagttaatagaaaaggcgatgaaattaaaataa

SEQ ID NO: 47 - DNA, 1602 bp, Bos taurus
atgcagggggcctgcgtgctgctgctgctgggcctgcatctacagctctccctaggcctcgtcccagttgaggaggaagaccccgccttctggaaccgccaggcagcccaggccctcgatgtggctaagaagctgcagcccatccagacagccgccaagaatgtcatcctcttcttgggggatgggatgggggtgcctacggtgacagccactcggatcctaaaggggcagatgaatggcaaactgggacctgagacacccctggccatggaccagttcccatacgtggctctgtccaagacatacaacgtggacagacaggtgccagacagcgcaggcactgccactgcctacctgtgtggggtcaagggcaactacagaaccattggtgtaagtgcagccgcccgctacaaccagtgcaaaacgacacgtgggaatgaggtcacgtctgtgatgaaccgggccaagaaagcagggaagtccgtgggagtggtgaccaccaccagggtgcagcatgcctccccagccggggcctacgcgcacacggtgaaccgaaactggtactcagacgccgacctgcctgctgatgcacagatgaatggctgccaggacatcgccgcacagctggtcaacaacatggatattgacgtgatcctgggtggaggccgaaaatacatgtttcctgtggggaccccagaccctgaatacccagatgatgccagtgtgaatggagtccggaagcgaaagcagaacctggtgcaggcatggcaggccaagcaccagggagcccagtatgtgtggaaccgcactgcgctccttcaggcggccgatgactccagtgtaacacacctcatgggcctctttgagccggcagacatgaagtataatgttcagcaagaccacaccaaggacccgaccctgcaggaaatgacagaggtggccctgcgagtcgtaagcaggaaccccaggggcttctacctctttgtggagggaggccgcattgaccacggtcaccatgatgacaaagcttatatggcactgaccgaggcgggtatgtttgacaatgccatcgccaaggctaatgagctcactagcgaactggacacgctgatccttgtcactgcagaccactctcatgtcttctcttttggtggctatacactgcgtgggacctccatttttggtctggcccccagcaaggccttagacagcaagtcctacacctccatcctctatggcaatggcccaggctatgcgcttggcgggggctcgaggcccgatgttaatgacagcacaagcgaggacccctcgtaccagcagcaggcggccgtgccccaggctagcgagacccacgggggcgaggacgtggcggtgttcgcgcgcggcccgcaggcgcacctggtgcacggcgtcgaggaggagaccttcgtggcgcacatcatggcctttgcgggctgcgtggagccctacaccgactgcaatctgccagcccccaccaccgccaccagcatccccgacgccgcgcacctggcggccagcccgcctccactggcgctgctggctggggcgatgctgctgctgctggcgcccaccttgtactaa

SEQ ID NO: 48 - DNA, 1506 bp, Thermus thermophilus
atgaagcgaagggacatcctgaaaggtggcctggctgcgggggccctggccctcctgccccggggccatacccagggggctctgcagaaccagccttccttggggaggcgctaccgcaacctgatcgtcttcgtctacgacgggttttcctgggaggactacgccatcgcccaggcctacgcccggaggaggcagggccgggttctcgccctggagcgcctcctcgcccgctaccccaacgggctcatcaacacctacagcctcaccagctacgtcaccgagtccagcgccgcggggaacgccttctcctgcggggtgaagacggtgaacggggggctcgccatccacgccgacgggacccccctcaagcccttcttcgccgcggccaaggaagcggggaaggccgtggggctcgtgaccaccaccaccgtcacccacgccaccccggcgagcttcgtgatctccaatcccgaccggaacgccgaggagaggatcgccgagcagtacctggagttcggggccgaggtgtacctggggggcggggaccgcttcttcaaccccgccaggcgcaaggacgggaaggacctctacgccgccttcgccgccaaggggtacggggtggtgcgcacccccgaggagctcgtccgttccaacgccacccggctcttgggcgtcttcgccgacggccacgtgccctacgagattgaccgccgcttccagggccttggggtgccgagcctcaaggaaatggtccaggccgctttgccccggcttgccgcccaccgcgggggcttcgtccttcaggtggaagcggggcggattgaccacgccaaccacttgaacgacgccggggccaccctttgggacgtgctggcggcggacgaggtcctggagctcctcaccgccttcgtggaccggaacccggacaccctcctcatcgtggtctcggaccacgccaccggggtaggggggctttacggggccgggcggagctacctggagagctcccaaggggtggacctcctggagccgcagcgggcgagctttgagcacatgctccgcgtcctcggccaggccccggaggcctcccaggtcaaggaggccttccgggccatgaagggggtggacctcgaggacgccgaggcggaaagggtggtgcgggccatccgggagaaggtctactggccggagggggtgcgccaaggggtccagcccgccaacaccatggcctgggccatggtgcagcgggacgcccagaagcccgaccggcccaacatcggctggtcctcggggcagcacacggcgagccccgtgatgctcctcctctacggccagggcctgcgctttgtgaacctgggcctcgtggacaacacccacgtcttccgcctcatgggggaggcccttggcctccgctaccagaacccggtgatgagcgaggaggaggccctggagatcctcaaggccaggccccaggggatgcgccaccccgaggacgtctgggcctaa

SEQ ID NO: 49 - DNA, 1428 bp, Pandalus borealis
  misc_feature (6)..(6): n is a, c, g, or t
aargcntaytggaayaargatgcccaagatgcactggataaacagctggggataaaactgcgagagaagcaggccaagaacgtgatcttcttcctaggagacgggatgtccctcagtacagttaccgcagcaagaatctacaaaggtggattgaccgggaaatttgaacgggaaaagatttcgtgggaagaattcgactttgctgctttgagtaagacgtacaatacggacaagcaagtgacagatagtgctgccagtgccacagcctacctcactggggtgaagaccaaccagggagtcatcggccttgatgccaataccgtgagaacaaactgctcttaccaacttgatgaatccctcttcacttactccatcgcccattggttccaggaggctggcagatcaacaggtgtcgtgacatcgaccagggtaactcatgctactcctgcaggaacttatgctcacgtggctgacagagattgggagaacgacagtgatgttgtccacgacagagaagatccagaaatatgtgatgacatagctgaacaactggtattcagagaacctggaaagaacttcaaggtcatcatgggcggcggtcggcgtggattctttcctgaggaagcgcttgatatcgaagatggcattccaggggaaagggaagatggcaaacacctcataaccgattggttggacgataaggcatctcagggagccacagcatcgtatgtctggaaccgagatgatctattggctgttgacattcgaaacacagactaccttatgggactgttttcatacacacatctagacacagttttaaccagagacgccgaaatggatcccaccttgccggaaatgacaaaggtcgccatcgaaatgctgacgaaggatgaaaatggctttttcctcttagtagaaggtggacggattgaccatatgcaccacgccaatcagatccgtcagtcgttagcggagacgctggacatggaggaagctgtatccatggctctctctatgacagacccagaggaaacaatcattctggtcacagctgatcacggtcacactctcaccatcactggatacgcagacaggaacaccgatattttggatttcgccggcatcagcgacttggatgataggaggtacaccatcctggactatggcagtggacctggatatcatatcacggaagatggcaaacgttacgaacctacagaggaagatttgaaagacatcaatttccgctacgcatcggcagcgcccaagcattcagttacccatgatggcacagacgttggtatttgggtcaacggacctttcgctcatctcttcacgggtgtctatgaagagaattacattccccacgccttggcctacgcagcctgcgttggtacaggacgcactttctgtgacgaaaaatga

SEQ ID NO: 50 - DNA, 1608 bp, Homo sapiens
atgctggggccctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcccatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccggggcggtccgtggtccccgcgttgcttcctctgctggccgggaccctgctgctgctggagacggccactgctccctga

SEQ ID NO: 51 - DNA, 1623 bp, Rattus norvegicus
atgcagggagactgggtgctgctgctgcttttgggtctcaggatacatctgtcctttggtgtcatcccagtggaggaggagaacccagtcttctggaatcaaaaggcgaaggaggccctggatgttgccaaaaagctgcagcccattcagacatcggccaagaacctcatcctcttcctgggagatgggatgggggtacccacagtgacagccaccaggatactaaagggacaactgggtggtcatctaggacctgagacacccctggccatggaccacttcccattcacggctctgtccaagacatacaacgtggacagacaggtcccagacagtgcaggcacagccaccgcctatctctgtggggtcaaagccaactacaagaccatcggtgtgagcgcggctgctcgattcaaccagtgcaacagcacatttggcaacgaggtcttctcagtgatgcaccgtgcaaagaaagcagggaagtctgtgggcgtggtgaccacgaccagagtgcagcatgcctctccagccggcacctacgcgcacacggttaaccgagactggtactcggatgcagacatgccttcctccgccctgcaggagggctgcaaggacatcgccacacagctcatttccaacatggacattgatgtgatccttggtggaggccgaaagtccatgtttcccaaggggacaccagaccctgaatacccaggtgactctgaccagtctggagtcaggctggatagtcggaacttggtggaggagtggctggcaaaataccaggggacccggtatgtttggaatcgagagcagctcatgcgggcctctcaggacccagcagtgactcgcctcatgggcctctttgagccaacagaaatgaaatatgatgtcaaccgaaacgcctcagcagacccctccctggcagagatgacagaggtggccgtgcgcctgctgagcaggaatccacaaggcttctacctctttgtagaaggaggtcgtattgaccaaggccaccatgcgggcacggcttacctggcactgactgaggctgccatgttcgactccgccatcgaaaaggccagccagcttaccaatgagaaggacacgctaacgctcatcactgcggaccactcccatgtctttgcttttggtggctatacacttagggggacctccatctttggtctggccccactcaatgcccaggatggcaaatcctacacctccattctctatggcaacggcccaggttatgtgcttaattcaggcaaccggcccaatgtcactgatgccgagagtggtgatgtcaactacaagcagcaggcagcagtacccctgtcgtcagagacccacggtggggaggacgtggccatattcgcgcgtggcccacaggcgcacctggtgcacggagtgcaggaacagaactacatcgcgcacgtcatggccttcgcaggctgcctggagccctacaccgactgcggtctggcgccccctgcagacgagaacagaccaaccaccccggtccagaacagtgccatcactatgaacaatgtccttctgtccctgcagctcctggtcagcatgctgctgctagtcgggacagccttggtggtgtcctga

SEQ ID NO: 52 - DNA, 1593 bp, Bos taurus
atgcagggggcctgcgtgctgctgctgctgggcctgtggctacagctctccctcgccttcatcccagttgaggaggaagaccccgccttctggaaccgccaggcagcccaggcccttgatgtggctaagaagctgcagcccatccagaaagccgccaagaatgtcatcctcttcttgggagatgggatgggggtgcctacggtgacagccactcggatactgaaggggcagatgaatgacaagctgggacctgagacacccctggccatggaccagttcccatacgtggctctgtccaagacatacaacgtggacagacaggtgccagacagcgcaggcactgccactgcctacctgtgtggggtcaagggcaactacagaaccatcggtgtaagtgcagccgcccgctacaatcagtgcaacacgacacgtgggaatgaggtcacgtctgtgatgaaccgggccaagaaagcagggaagtcagtgggagtggtgaccaccaccagggtgcagcacgcctccccagccggtgcttatgcacacacggtgaaccgtgactggtactcagacgccgacctgcctgccgatgcacagacgtatggctgccaggacatcgccacacaactggtcaacaacatggatattgacgtgatcctgggtggaggccgaaagtacatgtttcctgaggggaccccagaccctgaatacccacacgatgccagtgtgaatggagtccggaaggacaagcggaatctggtgcaggagtggcaggccaagcaccagggagcccagtatgtgtggaaccgcacggagctccttcaggcagccaatgactccagtgttacacatctcatgggcctctttgagccggcagacatgaagtataatgttcagcaagaccccaccaaggacccgaccctggaggagatgacggaggcggccctgcaagtgctgagcaggaacccccagggcttctacctcttcgtggagggaggccgcattgaccacggtcaccatgatagcaaagcttatatggcgctgactgaggcggtcatgtttgacaatgccatcgccaaggctaacgagctcactagcgaactggacacgctgatccttgtcactgcagaccactcccatgtcttctcttttggtggctacacactgcgtgggacctccattttcggtctggcccccagcaaggcctcagacaagaagtcctacacctccatcctctatggcaatggccctggctacgtgcttggtgggggctcaaggcccgatgttaatgacagcataagcgaggacccctcataccggcagcaggcggccgtgcccctgtctagcgagacccacgggggcgaagacgtggcggtgttcgcgcgaggcccgcaggcgcacctggtgcacggcgtgcaggaggagaccttcgtggcgcacgtcatggcctttgcgggctgcgtggagccctacaccgactgcaatctgccggccccctctggcctctccgacgccgcgcacctggcggccagcgcgccttcgctagcgctgctggccggggcgatgctgctgctgctggcgcccgccttgtactga

SEQ ID NO: 53 - DNA, 1680 bp, Mus musculus
atgcagggaccctgggtgctgctgctgctgggcctcaggctacagctgtcccttagtgtcattccagtggaggaggagaacccggccttctggaacaagaaggcagccgaggccctggatgctgccaagaagctgcagcccattcagacatcagctaagaacctcatcatcttcctgggtgacggaatgggggtaccaacagtgacagccaccaggatcctaaagggacagttggaaggtcatctaggacctgagacacccctagccatggaccgcttcccatatatggctctgtccaagacatacagtgtggacagacaggttccagacagtgcaagcacggccaccgcctacctgtgtggggtcaagaccaactacaagaccatcggcttgagtgcagccgcgagattcgaccagtgcaacaccacatttggcaatgaggtcttctcagtgatgtaccgtgccaagaaagcaggaaaatccgtaggtgtggtgaccaccaccagagtgcagcacgcctctccctcgggcacatatgttcacacagtgaaccgcaattggtatggggatgctgacatgcctgcctctgcgctgcgggaaggttgcaaggacattgctacacaactcatctccaacatggacattaatgtgatccttggtggggggcgaaaatacatgtttcctgctggaaccccagaccccgagtatccaaatgatgctaatgagactggaaccagattggatggcaggaatctggtgcaggaatggctgtcaaagcaccagggatcccagtatgtttggaatcgtgaacaactcattcagaaggcccaggatccgtcagtgacatacctcatgggcctctttgagcctgtagacacaaaatttgatattcaacgagatcccctgatggacccatctctgaaggatatgacagagacggccgtgaaagtgctaagcaggaaccccaaaggcttttatctctttgtggaggggggccgaatcgaccgtggtcaccatctgggcacagcttatctggcgctgactgaggctgtgatgttcgacttagccatcgagagggccagccagctcactagtgaacgcgacactctgaccatagtcactgctgaccactcccatgtcttctcctttggtggctacacacttcgagggacctccatcttcgggctggctcccctcaatgctctggacggcaagccctacacctccatcctgtatggcaacggcccaggctatgtcggtacaggggaaagacccaacgtcaccgccgctgaaagcagtggctcatcgtaccgcaggcaggctgctgtgccggtgaagtcggagacccacggcggggaggacgtggcgatattcgcgcgtggcccgcaggcgcacttggtgcacggggtgcaggagcagaactacatcgcgcacgtcatggcctctgcaggctgcctggagccctacaccgactgcggcttggcaccccctgcagatgaaagccagaccaccacgacaacccgccagaccaccatcaccaccaccaccaccaccaccaccaccacaaccaccccggtccataacagcgccagaagcctgggcccagccaccgccccgctggctctggcgctgctggccggaatgctgatgctactactaggggctcctgcggagtcctaa

SEQ ID NO: 54 - DNA, 300 bp, Streptococcus pneumoniae
atggcaaacaaacaagatttgatcgctaaagtagcagaagctacagaattgactaagaaagactcagcagcagcagttgaagctgtatttgcagcagtagctgactatcttgcagctggtgaaaaagttcaattgatcggttttggtaactttgaagttcgtgagcgtgcagaacgtaaaggtcgcaacccacaaactggtaaagaaatgacaattgcagcttctaaagtaccagcattcaaagctggtaaagctcttaaagacgctgttaaactcgagcaccaccaccaccaccactga

SEQ ID NO: 55 - DNA, 1494 bp, Thermococcus sp.
atgaaaggaaagtctcttgttagcggtctgttgttgggtcttttaattttgagcctgatttcattccagccaagctttgcatactccccacacggcggtgtcaaaaacatcataatcctggttggagacggcatgggtcttgggcatgtagaaattacaaagctcgtttatggacacttaaacatggaaaactttccagttactggatttgagcttactgattccctaagtggtgaagttacagattctgctgcggcaggaactgcaatatccactggagctaaaacgtataatggtatgatttcagtaaccaacataaccggaaagatagttaacttaacaaccctacttgaagtggctcaagagcttgggaagtcaacagggctggtcaccacaacaaggattacccatgcaactccagcagtttttgcgtcccatgtcccagatagggatatggagggggagatacccaagcaactcataatgcacaaagttaacgtcttgttgggtggtggaagggagaaattcgatgagaaaaatttggagctggccaaaaagcagggatacaaagtagttttcacgaaggaagagcttgaaaaagttgaaggagattatgtcctaggactctttgcagaaagtcacatcccttacgtattggatagaaaacccgatgatgttggacttttagaaatggccaaaaaggcaatttcaatactcgagaagaacccgagcggattctttctcatggttgagggcggaaggattgaccatgcagcccatggaaacgatgtcgcatcggttgttgcagaaactaaggagtttgacgatgttgtcagatacgtgctggaatatccgaagaagaggggagataccttggtaatagtgcttgccgatcacgaaactggaggtcttgcaataggtctaacgtatggaaatgcaatcgatgaagatgccataagaaaaataaaagcaagcacgttgaggatgcccaaagaggttaaggcagggagtagtgtaaaagagtcctcaaaggtatgccggatttgtcccaacagaggaagaagtcagtatattgagaatgcgctgcactcgacaaacaagtatgccctctcaaatgcagtagccgatgttataaacaggcgtattggtgttggattcacctcctatgagcatacaggagttccagttccgctcttagcttacggtcccggggcagagaacttcagaggtttcttacaccatgtggatacagcaagattagttgcaaagttaatgctctttggaaggaggaatattccagttaccatttcaagcgtgagcagtgttaagggagacataaccggtgattacagggttgatgagaaggatgcctacgttacgctcatgatgtttctcggagaaaaagtggataatgaaattgaaaagagagtcgatatagacaacaacggcatggttgacttaaatgacgtcatgttgattctccaggaagcttga

SEQ ID NO: 56 - DNA, 1778 bp, Bacillus subtilis
tttttatttaaacaaacatatcatgcaaagacagagaggtaaagatttttctgaaaaatgaatgctttacatcaaataaggcaagataacgaaaagcgttttttcatttccttacaaggctttcattattgtttacatgatcaacagccgcatttaacaaagtttccctaacatgataaacggaatacattaaaggaggcatgaaaaaaatgagtttgtttcaaaatatgaaatcaaaacttctgccaatcgccgctgtttctgtccttacagctggaatctttgccggagctgagcttcagcaaacagaaaaggccagcgccaaaaaacaagacaaagctgagatcagaaatgtcattgtgatgataggcgacggcatggggacgccttacataagagcctaccgttccatgaaaaataacggtgacacaccgaataacccgaagttaacagaatttgaccggaacctgacaggcatgatgatgacgcatccggatgaccctgactataatattacagattcagcagcagccggaacagcattagcgacaggcgttaagacatataacaatgcaattggcgtcgataaaaacggaaaaaaagtgaaatctgtacttgaagaggccaaacagcaaggcaagtcaacagggcttgtcgccacgtctgaaattaaccacgccactccagccgcatatggcgcccacaatgaatcacggaaaaacatggaccaaatcgccaacagctatatggatgacaagataaaaggcaaacataaaatagacgtgctgctcggcggcggaaaatcttattttaaccgcaagaacagaaacttgacaaaggaattcaaacaagccggctacagctatgtgacaactaaacaagcattgaaaaaaaataaagatcagcaggtgctcgggcttttcgcagatggagggcttgctaaagcgctcgaccgtgacagtaaaacaccgtctctcaaagacatgacggtttcagcaattgatcgcctgaaccaaaataaaaaaggatttttcttgatggtcgaagggagccagattgactgggcggcccatgacaatgatacagtaggagccatgagcgaggttaaagattttgagcaggcctataaagccgcgattgaatttgcgaaaaaagacaaacatacacttgtgattgcaactgctgaccatacaaccggcggctttaccattggcgcaaacggggaaaagaattggcacgcagaaccgattctctccgctaagaaaacacctgaattcatggccaaaaaaatcagtgaaggcaagccggttaaagatgtgctcgcccgctatgccaatctgaaagtcacatctgaagaaatcaaaagcgttgaagcagctgcacaggctgacaaaagcaaaggggcctccaaagccatcatcaagatttttaatacccgctccaacagcggatggacgagtaccgatcataccggcgaagaagtaccggtatacgcgtacggccccggaaaagaaaaattccgcggattgattaacaatacggaccaggcaaacatcatatttaagattttaaaaactggaaaataaaaaggatgcagaacgctgcatcctcttttttataaatatctgcggtgaacaccttttccgtcgtacgaaaacatgacttccttatcctcaagcacagactcaatgtgcacgcttcttccccaaagctggaataaataaggaagcactttctccaaatatttcaaatccaactctatgccctcataccaatgc

SEQ ID NO: 57 - DNA, 1602 bp, Bos taurus; identical to SEQ ID NO: 47

SEQ ID NO: 58 - protein, 433 aa, Thermotoga neapolitana; identical to SEQ ID NO: 2

SEQ ID NO: 59 - protein, 434 aa, Thermotoga maritima
MKRLFTLILISLLVALAFSQVKNVIYLIGDGMGLSQVYLTSMLEGRPLSFMKTPYIGLVKTHSANSWVTDSAAAGTALASGFKTNNGMINILPDGTVVPTIFEVAKTYGVRTGIVVTCRVTHATPAAFYAHVKSRDEENEIARQLVENETVDVVMGGGWANFLPKDLGGKRDDNLNLIELAKEKGYIYVKTREELSKISADSDKILALFAPSHLDPASSRKEQPMLYEMVEKALEILSKDDEPFFLMVEGSQIDWEAHDNDIYGVWKEVVEFDKAVQVALDFALKRGDTLVIVTADHETGGLGLSSGDYRVDVDKIRNFKKTTDWIMANYSPKDREKFKKAIEEYFGLTLSDEDLNRISMSKNPKIELGRILGEKVSVGWTTTTHSGTPVPIFAFGPGAENFTGFLDNTEIPRIIMKLTGYSLQYPLLKEPVTK

SEQ ID NO: 60 - protein, 434 aa, Thermotoga naphthophila
MKRLFTLILISLLVALVFSQVKNVIYFIGDGMGLSQVYLTSMLEGRPLSLMKTPYIGLVKTHSANSWVTDSAAAGTALASGFKTNNGMINILPDGTVVPTIFEVAKTYGVRTGIVVTCRVTHATPAAFYAHVKSRDEENEIARQLVENETVDVVMGGGWANFLPKDLGGKRDDNLNLIELAKEKGYIYVKTREELSKISADSDKILALFAPSHLDPASSRKEQPMLYEMVEKVLEILSKDDEPFFLMVEGSQIDWEAHDNDIYGVWKEVKEFDKAVQVALDFALKRGDTLVIVTADHETGGLGLSSGDYRVDVDKIRNFKKTTDWIMANYSPKDQEKFKKAIEEYFGLTLSDEDLNRISMSKNPKIELGRILGEKVSVGWTTTTHSGTPVPIFAFGPGAENFTGFLDNTEIPRIIMKLTGYSLQYPLLKEPVTK

SEQ ID NO: 61 - protein, 444 aa, Bacillus halodurans
MKHRVHVTVLLFVMIVLILPAETVFASFVQEGTITPDSEQVKNIIYMIPDGYSASYATNYRIYKGEEEPIWDPHLVGMVKTHSADSWVTDSAAAGTALATGTKTSNGTIGMSTEGEELESILQAAGKQKKGTGIVVTTRLTHATPAAFVASVPLRIDEAKIAPQMIGQVDVLLGGGKKMFVPEAEGGMQNSRHLLNEAEDAGYALIETKEELQNVNSAKVLGLFSEGALAPELDRQGTQEPSLAEMTEAALHTLKQNPNGFFLMVEGSQIDWAGHDNDAAWAMKDIEAFEEAVSVAFDFAEKDGNTLVVVVGDHDTGGLSVGAYGQYKAKPDMLKQVKATGKFIASQVNKRRTNLADVMKTYTGFELTKKELKQIKKADHLTNAINQVISRRALIGWTTPVHTGTDIPIYAFGPCSDRLHGLLDNTDIPLILADVMDLEWEKAS

SEQ ID NO: 62 - protein, 471 aa, Escherichia coli
MKQSTIALALLPLLFTPVTKARTPEMPVLENRAAQGDITAPGGARRLTGDQTAALRDSLSDKPAKNIILLIGDGMGDSEITAARNYAEGAGGFFKGIDALPLTGQYTHYALNKKTGKPDYVTDSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLK

SEQ ID NO: 63 - protein, 471 aa, Escherichia coli
MKQSTIALALLPLLFTPVTKARTPEMPVLENRAAQSDITAPGGARRLTGDQTAALRDSLSDKPAKNIILLIGDGMGDSEITAARNYAEGAGGFFKGIDALPLTGQYTHYALNKKTGKPDYVTDSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWQGPKATYHGNIDKPAVTCTPNPQRNDSVPTLAQMTDKAIELLSKNEKGFFLQVEGASIDKQDHAANPCGAIGETVDLDEAVQRALEFAKKDGNTLVIVTADHAHASQIVAPDTKAPGLTQALNTKDGAVMVMSYGNSEEDSQEHTGSQLRIAAYGPHAANVVGLTDQTDLFYTMKAALGLK

SEQ ID NO: 64 - protein, 446 aa, Bacillus clausii
MKRSKKLAVAGLVGIVCLGATYTAAFASDSGEVGEEGNQPSSGQAKNVIFLIPDGFSQGYTNNYRLYKEDGEPIWDERNMLRAFVQTHSANAEVTDSAAAGTALATGEKTNNGMIGVTPAGQTLPTILDSAKENGKRTGLVATSTITHATPAAFAVSVESRNSYTEIASQMVANDHIDLMLGGGRTEFVPEDQGGIREDGVDLISEAEEKGFAFVETARQLANWNGEEDRLLGLFAEEALSPAANKRTDEPSLGEMTEKAIEFLSKEDNGFFLMVEGSQIDWAGHANDAYYAMTDTAAFEEAVEAAVDFADENGETLVVMVGDHDTGGMAVHASEEGDPSKLHGVSALGSDMAAEVERNYSNLKEVLETYTNFEWTEEELDQLKESDSLELAINTAISEKAGVAWSSLDHTGIDVPLYAYGAGAERFTGTIDNTEVPKIMKAALGI

SEQ ID NO: 65 - protein, 443 aa, Oceanobacillus iheyensis
MLKKVGWISVISLLIFAGIMGASFGNEEDATSAKGNNKKVENVIYMIPDGFSSDYASNYRAYKGEEVVWDSHLKGLFTTHSADSDVTDSAAAGTAMATGEKTNNGVIGKDTNGNNLETILEKSKQHHKATGLVATSTITHATPAAFATHVEDRNNETEIARQLIKSEVDVLLGGGMNKFIGEEEGGNQEDGELMQKAQDTGYILAENKQEMLEQNIDIKKDEKLLGLFADEALSPELHRKDTEEPSLEDMTTHAIDQLNQNKKGFFLMVEGSQIDWAGHDNDSAYAMSDTEAFEKAVQAAIDFAEEDGETLVVVAADHDTGGMTTGGYDQMELNANILNDVTATGEYMANQLDEERSNVHEVVNTYTGFKLTEEEIESIETAEDPKLAINHVVSARSTIGWTSTGHTAADIPIYAYGPSAESFSGKLDNTDIPKLIEKAMKLK

SEQ ID NO: 66 - protein, 501 aa, Thermus thermophilus
MKRRDILKGGLAAGALALLPRGHTQGALQNQPSLGRRYRNLIVFVYDGFSWEDYAIAQAYARRRQGRVLALERLLARYPNGLINTYSLTSYVTESSAAGNAFSCGVKTVNGGLAIHADGTPLKPFFAAAKEAGKAVGLVTTTTVTHATPASFVISNPDRNAEERIAEQYLEFGAEVYLGGGDRFFNPARRKDGKDLYAAFAAKGYGVVRTPEELVRSNATRLLGVFADGHVPYEIDRRFQGLGVPSLKEMVQAALPRLAAHRGGFVLQVEAGRIDHANHLNDAGATLWDVLAADEVLELLTAFVDRNPDTLLIVVSDHATGVGGLYGAGRSYLESSQGVDLLEPQRASFEHMLRVLGQAPEASQVKEAFRAMKGVDLEDAEAERVVRAIREKVYWPEGVRQGVQPANTMAWAMVQRDAQKPDRPNIGWSSGQHTASPVMLLLYGQGLRFVNLGLVDNTHVFRLMGEALGLRYQNPVMSEEEALEILKARPQGMRHPEDVWA

SEQ ID NO: 67 - protein, 533 aa, Bos taurus
MQGACVLLLLGLHLQLSLGLVPVEEEDPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCKTTRGNEVTSVMNRAKKAGKSVGVVTTTRVQHASPAGAYAHTVNRNWYSDADLPADAQMNGCQDIAAQLVNNMDIDVILGGGRKYMFPVGTPDPEYPDDASVNGVRKRKQNLVQAWQAKHQGAQYVWNRTALLQAADDSSVTHLMGLFEPADMKYNVQQDHTKDPTLQEMTEVALRVVSRNPRGFYLFVEGGRIDHGHHDDKAYMALTEAGMFDNAIAKANELTSELDTLILVTADHSHVFSFGGYTLRGTSIFGLAPSKALDSKSYTSILYGNGPGYALGGGSRPDVNDSTSEDPSYQQQAAVPQASETHGGEDVAVFARGPQAHLVHGVEEETFVAHIMAFAGCVEPYTDCNLPAPTTATSIPDAAHLAASPPPLALLAGAMLLLLAPTLY

SEQ ID NO: 68 - protein, 475 aa, Pandalus borealis
KAYWNKDAQDALDKQLGIKLREKQAKNVIFFLGDGMSLSTVTAARIYKGGLTGKFEREKISWEEFDFAALSKTYNTDKQVTDSAASATAYLTGVKTNQGVIGLDANTVRTNCSYQLDESLFTYSIAHWFQEAGRSTGVVTSTRVTHATPAGTYAHVADRDWENDSDVVHDREDPEICDDIAEQLVFREPGKNFKVIMGGGRRGFFPEEALDIEDGIPGEREDGKHLITDWLDDKASQGATASYVWNRDDLLAVDIRNTDYLMGLFSYTHLDTVLTRDAEMDPTLPEMTKVAIEMLTKDENGFFLLVEGGRIDHMHHANQIRQSLAETLDMEEAVSMALSMTDPEETIILVTADHGHTLTITGYADRNTDILDFAGISDLDDRRYTILDYGSGPGYHITEDGKRYEPTEEDLKDINFRYASAAPKHSVTHDGTDVGIWVNGPFAHLFTGVYEENYIPHALAYAACVGTGRTFCDEK

SEQ ID NO: 69 - protein, 497 aa, Thermococcus sp.
MKGKSLVSGLLLGLLILSLISFQPSFAYSPHGGVKNIIILVGDGMGLGHVEITKLVYGHLNMENFPVTGFELTDSLSGEVTDSAAAGTAISTGAKTYNGMISVTNITGKIVNLTTLLEVAQELGKSTGLVTTTRITHATPAVFASHVPDRDMEGEIPKQLIMHKVNVLLGGGREKFDEKNLELAKKQGYKVVFTKEELEKVEGDYVLGLFAESHIPYVLDRKPDDVGLLEMAKKAISILEKNPSGFFLMVEGGRIDHAAHGNDVASVVAETKEFDDVVRYVLEYPKKRGDTLVIVLADHETGGLAIGLTYGNAIDEDAIRKIKASTLRMPKEVKAGSSVKESSKVCRICPNRGRSQYIENALHSTNKYALSNAVADVINRRIGVGFTSYEHTGVPVPLLAYGPGAENFRGFLHHVDTARLVAKLMLFGRRNIPVTISSVSSVKGDITGDYRVDEKDAYVTLMMFLGEKVDNEIEKRVDIDNNGMVDLNDVMLILQEA

SEQ ID NO: 70 - protein, 461 aa, Bacillus subtilis
MKKMSLFQNMKSKLLPIAAVSVLTAGIFAGAELQQTEKASAKKQDKAEIRNVIVMIGDGMGTPYIRAYRSMKNNGDTPNNPKLTEFDRNLTGMMMTHPDDPDYNITDSAAAGTALATGVKTYNNAIGVDKNGKKVKSVLEEAKQQGKSTGLVATSEINHATPAAYGAHNESRKNMDQIANSYMDDKIKGKHKIDVLLGGGKSYFNRKNRNLTKEFKQAGYSYVTTKQALKKNKDQQVLGLFADGGLAKALDRDSKTPSLKDMTVSAIDRLNQNKKGFFLMVEGSQIDWAAHDNDTVGAMSEVKDFEQAYKAAIEFAKKDKHTLVIATADHTTGGFTIGANGEKNWHAEPILSAKKTPEFMAKKISEGKPVKDVLARYANLKVTSEEIKSVEAAAQADKSKGASKAIIKIFNTRSNSGWTSTDHTGEEVPVYAYGPGKEKFRGLINNTDQANIIFKILKTGK

SEQ ID NO: 71 - protein, 535 aa, Homo sapiens
MLGPCMLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGRSVVPALLPLLAGTLLLLETATAP

SEQ ID NO: 72 - protein, 540 aa, Rattus norvegicus
MQGDWVLLLLLGLRIHLSFGVIPVEEENPVFWNQKAKEALDVAKKLQPIQTSAKNLILFLGDGMGVPTVTATRILKGQLGGHLGPETPLAMDHFPFTALSKTYNVDRQVPDSAGTATAYLCGVKANYKTIGVSAAARFNQCNSTFGNEVFSVMHRAKKAGKSVGVVTTTRVQHASPAGTYAHTVNRDWYSDADMPSSALQEGCKDIATQLISNMDIDVILGGGRKSMFPKGTPDPEYPGDSDQSGVRLDSRNLVEEWLAKYQGTRYVWNREQLMRASQDPAVTRLMGLFEPTEMKYDVNRNASADPSLAEMTEVAVRLLSRNPQGFYLFVEGGRIDQGHHAGTAYLALTEAAMFDSAIEKASQLTNEKDTLTLITADHSHVFAFGGYTLRGTSIFGLAPLNAQDGKSYTSILYGNGPGYVLNSGNRPNVTDAESGDVNYKQQAAVPLSSETHGGEDVAIFARGPQAHLVHGVQEQNYIAHVMAFAGCLEPYTDCGLAPPADENRPTTPVQNSAITMNNVLLSLQLLVSMLLLVGTALVVS

SEQ ID NO: 73 - protein, 530 aa, Bos taurus
MQGACVLLLLGLWLQLSLAFIPVEEEDPAFWNRQAAQALDVAKKLQPIQKAAKNVILFLGDGMGVPTVTATRILKGQMNDKLGPETPLAMDQFPYVALSKTYNVDRQVPDSAGTATAYLCGVKGNYRTIGVSAAARYNQCNTTRGNEVTSVMNRAKKAGKSVGVVTTTRVQHASPAGAYAHTVNRDWYSDADLPADAQTYGCQDIATQLVNNMDIDVILGGGRKYMFPEGTPDPEYPHDASVNGVRKDKRNLVQEWQAKHQGAQYVWNRTELLQAANDSSVTHLMGLFEPADMKYNVQQDPTKDPTLEEMTEAALQVLSRNPQGFYLFVEGGRIDHGHHDSKAYMALTEAVMFDNAIAKANELTSELDTLILVTADHSHVFSFGGYTLRGTSIFGLAPSKASDKKSYTSILYGNGPGYVLGGGSRPDVNDSISEDPSYRQQAAVPLSSETHGGEDVAVFARGPQAHLVHGVQEETFVAHVMAFAGCVEPYTDCNLPAPSGLSDAAHLAASAPSLALLAGAMLLLLAPALY

SEQ ID NO: 74 - protein, 559 aa, Mus musculus
MQGPWVLLLLGLRLQLSLSVIPVEEENPAFWNKKAAEALDAAKKLQPIQTSAKNLIIFLGDGMGVPTVTATRILKGQLEGHLGPETPLAMDRFPYMALSKTYSVDRQVPDSASTATAYLCGVKTNYKTIGLSAAARFDQCNTTFGNEVFSVMYRAKKAGKSVGVVTTTRVQHASPSGTYVHTVNRNWYGDADMPASALREGCKDIATQLISNMDINVILGGGRKYMFPAGTPDPEYPNDANETGTRLDGRNLVQEWLSKHQGSQYVWNREQLIQKAQDPSVTYLMGLFEPVDTKFDIQRDPLMDPSLKDMTETAVKVLSRNPKGFYLFVEGGRIDRGHHLGTAYLALTEAVMFDLAIERASQLTSERDTLTIVTADHSHVFSFGGYTLRGTSIFGLAPLNALDGKPYTSILYGNGPGYVGTGERPNVTAAESSGSSYRRQAAVPVKSETHGGEDVAIFARGPQAHLVHGVQEQNYIAHVMASAGCLEPYTDCGLAPPADESQTTTTTRQTTITTTTTTTTTTTTPVHNSARSLGPATAPLALALLAGMLMLLLGAPAES

SEQ ID NO: 75 - DNA, 270 bp, Escherichia coli
atgggtggtatcagtatttggcagttattgattattgccgtcatcgttgtactgctttttggcaccaaaaagctcggctccatcggttccgatcttggtgcgtcgatcaaaggctttaaaaaagcaatgagcgatgatgaaccaaagcaggataaaaccagtcaggatgctgattttactgcgaaaactatcgccgataagcaggcggatacgaatcaggaacaggctaaaacagaagacgcgaagcgccacgataaagagcaggtgtaa

SEQ ID NO: 76 - protein, 89 aa, Escherichia coli
MGGISIWQLLIIAVIVVLLFGTKKLGSIGSDLGASIKGFKKAMSDDEPKQDKTSQDADFTAKTIADKQADTNQEQAKTEDAKRHDKEQV

SEQ ID NO: 77 - protein, 204 aa, Escherichia coli
MTHFSQQDNFSVAARVLGALFYYAPESAEAAPLVAVLTSDGWETQWPLPEASLAPLVTAFQTQCEETHAQAWQRLFVGPWALPSPPWGSVWLDRESVLFGDSTLALRQWMREKGIQFEMKQNEPEDHFGSLLLMAAWLAENGRQTECEELLAWHLFPWSTRFLDVFIEKAEHPFYRALGELARLTLAQWQSQLLIPVAVKPLFR

SEQ ID NO: 78 - DNA, 615 bp, Escherichia coli
atgacccatttttcacagcaagataatttttctgtcgcggcgcgcgtgttgggcgcgctgttttattacgctccagagagtgcggaagccgcacctctggttgcggtactcaccagtgacggctgggaaactcagtggcctctaccagaagcgtcattagcgccgctggtgacggcttttcagacacagtgtgaagagactcacgcccaggcctggcagcgtttgtttgtcggcccgtgggcactgccgtctccgccgtgggggtcggtctggctggatcgcgaatctgtgctgtttggcgattcaacattggcacttcgtcagtggatgcgcgagaaaggcattcagtttgaaatgaagcaaaacgaaccggaagatcattttggttcactgctgttgatggctgcgtggttggcggagaatggtcgccagacagaatgcgaagaattgctggcatggcacctttttccgtggtcaacacgttttcttgatgtttttatcgaaaaagcagaacaccctttctaccgtgcactgggtgaactggctcgcttaacgctggcgcaatggcaatcacaactgttaattcctgtcgcggttaaaccgctgtttcgatag

SEQ ID NO: 79 - DNA, 777 bp, Escherichia coli
atgtctgtagaagatactcaaccgcttatcacgcatctgattgagctgcgtaagcgtctgctgaactgcattatcgcggtgatcgtgatattcctgtgtctggtctatttcgccaatgacatctatcacctggtatccgcgccattgatcaagcagttgccgcaaggttcaacgatgatcgccaccgacgtggcctcgccgttctttacgccgatcaagctgacctttatggtgtcgctgattctgtcagcgccggtgattctctatcaggtgtgggcatttatcgccccagcgctgtataagcatgaacgtcgcctggtggtgccgctgctggtttccagctctctgctgttttatatcggcatggcattcgcctactttgtggtctttccgctggcatttggcttccttgccaataccgcgccggaaggggtgcaggtatccaccgacatcgccagctatttaagcttcgttatggcgctgtttatggcgtttggtgtctcctttgaagtgccggtagcaattgtgctgctgtgctggatggggattacctcgccagaagacttacgcaaaaaacgcccgtatgtgctggttggtgcattcgttgtcgggatgttgctgacgccgccggatgtcttctcgcaaacgctgttggcgatcccgatgtactgtctgtttgaaatcggtgtcttcttctcacgcttttacgttggtaaagggcgaaatcgggaagaggaaaacgacgctgaagcagaaagcgaaaaaactgaagaataa

SEQ ID NO: 80 - protein, 258 aa, Escherichia coli
MSVEDTQPLITHLIELRKRLLNCIIAVIVIFLCLVYFANDIYHLVSAPLIKQLPQGSTMIATDVASPFFTPIKLTFMVSLILSAPVILYQVWAFIAPALYKHERRLVVPLLVSSSLLFYIGMAFAYFVVFPLAFGFLANTAPEGVQVSTDIASYLSFVMALFMAFGVSFEVPVAIVLLCWMGITSPEDLRKKRPYVLVGAFVVGMLLTPPDVFSQTLLAIPMYCLFEIGVFFSRFYVGKGRNREEENDAEAESEKTEE

SEQ ID NO: 81 - protein, 550 aa, Escherichia coli
MAAKDVKFGNDARVKMLRGVNVLADAVKVTLGPKGRNVVLDKSFGFTAPTITKDGVSVAREIELEDKFENMGAQMVKEVASKANDAAGDGTTTATVLAQAIITEGLKAVAAGMNPMDLKRGIDKAVTAAVEELKALSVPCSDSKAIAQVGTISANSDETVGKLIAEAMDKVGKEGVITVEDGTGLQDELDVVEGMQFDRGYLSPYFINKPETGAVELESPFILLADKKISNIREMLPVLEAVAKAGKPLLIIAEDVEGEALATAVVNTIRGIVKVAAVKAPGFGDRRKAMLQDIATLTGGTVISEEIGMELEKATLEDLGQAKRVVINKDTTTIIDGVGEEAAIQGRVAQIRQQIEEATSDYDREKLQERVAKLAGGVAVIKVGAATEVEMKEKKARVEDALHATRAAVEEGVVAGGGVALIRVASKLADLRGQNEDQNVGIKVALRAMEAPLRQIVLNCGEEPSVVANTVKGGDGNYGYNAATEEYGNMIDMGILDPTKVTRSALQYAASVAGLMITTECMVTDLPKNDAADLGAAGGMGGMGGMGGMM

SEQ ID NO: 82 - DNA, 1647 bp, Escherichia coli
atggcagctaaagacgtaaaattcggtaacgacgctcgtgtgaaaatgctgcgcggcgtaaacgtactggcagatgcagtgaaagttaccctcggtccaaaaggccgtaacgtagttctggataaatctttcggtgcaccgaccatcaccaaagatggtgtttccgttgctcgtgaaatcgaactggaagacaagttcgaaaatatgggtgcgcagatggtgaaagaagttgcctctaaagcaaacgacgctgcaggcgacggtaccaccactgcaaccgtactggctcaggctatcatcactgaaggtctgaaagctgttgctgcgggcatgaacccgatggacctgaaacgtggtatcgacaaagcggttaccgctgcagttgaagaactgaaagcgctgtccgtaccatgctctgactctaaagcgattgctcaggttggtaccatctccgctaactccgacgaaaccgtaggtaaactgatcgctgaagcgatggacaaagtcggtaaagaaggcgttatcaccgttgaagacggtaccggtctgcaggacgaactggacgtggttgaaggtatgcagttcgaccgtggctacctgtctccttacttcatcaacaagccggaaactggcgcagtagaactggaaagcccgttcatcctgctggctgacaagaaaatctccaacatccgcgaaatgctgccggttctggaagctgttgccaaagcaggcaaaccgctgctgatcatcgctgaagatgtagaaggcgaagcgctggcaactgctgttgttaacaccattcgtggcatcgtgaaagtcgctgcggttaaagcaccgggcttcggcgatcgtcgtaaagctatgctgcaggatatcgcaaccctgactggcggtaccgtgatctctgaagagatcggtatggagctggaaaaagcaaccctggaagacctgggtcaggctaaacgtgttgtgatcaacaaagacaccaccactatcatcgatggcgtgggtgaagaagctgcaatccagggccgtgttgctcagatccgtcagcagattgaagaagcaacttctgactacgaccgtgaaaaactgcaggaacgcgtagcgaaactggcaggcggcgttgcagttatcaaagtgggtgctgctaccgaagttgaaatgaaagagaaaaaagcacgcgttgaagatgccctgcacgcgacccgtgctgcggtagaagaaggcgtggttgctggtggtggtgttgcgctgatccgcgtagcgtctaaactggctgacctgcgtggtcagaacgaagaccagaacgtgggtatcaaagttgcactgcgtgcaatggaagctccgctgcgtcagatcgtattgaactgcggcgaagaaccgtctgttgttgctaacaccgttaaaggcggcgacggcaactacggttacaacgcagcaaccgaagaatacggcaacatgatcgacatgggtatcctggatccaaccaaagtaactcgttctgctctgcagtacgcagcttctgtggctggcctgatgatcaccaccgaatgcatggttaccgacctgccgaaaaacgatgcagctgacttaggcgctgctggcggtatgggcggcatgggtggcatgggcggcatgatgtaa

SEQ ID NO: 83 - protein, 97 aa, Escherichia coli [sequence omitted]

SEQ ID NO: 84 - DNA, 294 bp, Escherichia coli
atgaatattcgtccattgcatgatcgcgtgatcgtcaagcgtaaagaagttgaaactaaatctgctggcggcatcgttctgaccggctctgcagcggctaaatccacccgcggcgaagtgctggctgtcggcaatggccgtatccttgaaaatggcgaagtgaagccgctggatgtgaaagttggcgacatcgttattttcaacgatggctacggtgtgaaatctgagaagatcgacaatgaagaagtgttgatcatgtccgaaagcgacattctggcaattgttgaagcgtaa

SEQ ID NO: 85 - protein, 155 aa, Escherichia coli
MSEQNNTEMTFQIQRIYTKDISFEAPNAPHVFQKDWQPEVKLDLDTASSQLADDVYEVVLRVTVTASLGEETAFLCEVQQGGIFSIAGIEGTQMAHCLGAYCPNILFPYARECITSMVSRGTFPQLNLAPVNFDALFMNYLQQQAGEGTEEHQDA

SEQ ID NO: 86 - DNA, 468 bp, Escherichia coli
atgtcagaacaaaacaacactgaaatgactttccagatccaacgtatttataccaaggatatctctttcgaagcgccgaacgcgccgcacgttttccagaaagattggcaaccagaagttaaacttgatctggatacggcatcttcccaactggcagatgacgtatacgaagtggtactgcgtgttaccgtaacggcctctttgggcgaagaaaccgcgttcctgtgtgaagttcagcagggcggtattttctccatcgcgggtatcgaaggcacccagatggcgcattgcctgggagcatactgcccgaacattctgttcccgtatgctcgtgagtgcatcaccagcatggtatcccgcggtacattcccgcaactgaaccttgcgccggttaacttcgatgcgctgttcatgaactatttgcagcagcaggctggcgaaggtactgaagaacatcaggatgcctga

SEQ ID NO: 87 - DNA, 101 bp, Artificial Sequence
  Synthetic
agatctcgatcccgcgaaattaatacgactcactataggggaattgtgagcggataacaattcccctctagaaataattttgtttaactttaagaaggaga

SEQ ID NO: 88 - DNA, 110 bp, Artificial Sequence
  Synthetic
  CDS (8)..(109)
tatacatatggctagcatgactggtggacagcaaatgggtcgcggatccgaattcgagctccgtcgacaagcttgcggccgcactcgagcaccaccaccaccaccactga

SEQ ID NO: 89 - protein, 34 aa, Artificial Sequence
  Synthetic Construct
MASMTGGQQMGRGSEFELRRQACGRTRAPPPPPL

SEQ ID NO: 90 - DNA, 72 bp, Artificial Sequence
  Synthetic
  CDS (1)..(72)
ggtcgggatccgaattcgagctccgtcgacaagcttgcggccgcactcgagcaccaccaccaccaccactga

SEQ ID NO: 91 - protein, 23 aa, Artificial Sequence
  Synthetic Construct
GRDPNSSSVDKLAAALEHHHHHH

SEQ ID NO: 92 - DNA, 71 bp, Artificial Sequence
  Synthetic
  CDS (1)..(69)
ggtcggatccgaattcgagctccgtcgacaagcttgcggccgcactcgagcaccaccaccaccaccactga

SEQ ID NO: 93 - protein, 23 aa, Artificial Sequence
  Synthetic Construct
GRIRIRAPSTSLRPHSSTTTTTT

SEQ ID NO: 94 - DNA, 111 bp, Artificial Sequence
  Synthetic
gatccggctgctaacaaagcccgaaaggaagctgagttggctgctgccaccgctgagcaataactagcataaccccttggggcctctaaacgggtcttgaggggttttttg

SEQ ID NO: 95 - DNA, 13 bp, Artificial Sequence
  Synthetic
  CDS (5)..(13)
taccatggctagc

(19) United States
(12) Patent Application Publication
(10) Pub. No.: US 2008/0176283 A1
(43) Pub. Date: Jul. 24, 2008

(54) Thermotoga neapolitana DSM 5068 alkaline phosphatase (PhoA) gene and recombinant enzyme production

(75) Inventors: Claire Vieille, Lansing, MI (US); J. Gregory Zeikus, Okemos, MI (US); Maris Laivenieks, East Lansing, MI (US)

Correspondence Address: Medlen & Carroll, LLP, Suite 350, 101 Howard Street, San Francisco, CA, 94105, US

(73) Assignee: Michigan State University, Lansing, MI (US)

(21) Appl. No.: 11/906,045

(22) Filed: Sep. 28, 2007

Related U.S. Application Data
(60) Provisional application No. 60/847,718, filed on Sep. 28, 2006.

(51) Int. Cl. C12P 21/04; C12N 15/00; C12N 15/11

(52) U.S. Cl. 435/69.1; 435/320.1; 536/23.2

(57) ABSTRACT

The present invention relates to compositions and methods for providing a recombinant thermostable Thermotoga neapolitana alkaline phosphatase enzyme. More particularly, the invention relates to engineering Escherichia coli with T. neapolitana alkaline phosphatase gene expression vectors for providing an inducible system for thermostable enzyme production, wherein the expressed enzyme is readily soluble with a high degree of activity. These methods provide for commercial quantities of a thermostable alkaline phosphatase enzyme.

This invention was made in part with government support under grant MCB-0445750 from the National Science Foundation. As such, the United States Government has certain rights in the invention.

### FIELD OF THE INVENTION

The present invention relates to compositions and methods for providing a recombinant thermostable Thermotoga neapolitana alkaline phosphatase enzyme. More particularly, the invention relates to engineering Escherichia coli with T. neapolitana alkaline phosphatase gene expression vectors for providing an inducible system for thermostable enzyme production, wherein the expressed enzyme is readily soluble with a high degree of activity and stability. These methods provide for producing commercial quantities of a thermostable alkaline phosphatase enzyme.

### BACKGROUND

Alkaline phosphatase (AP) (orthophosphoric-monoester phosphohydrolase) (EC 3.1.3.1) is a non-specific phosphomonoesterase. This enzyme functions through a phosphoseryl intermediate (Engstrom, (1962) Biochim. Biophys. Acta 56:606-609; herein incorporated by reference) that can produce either an alcohol and inorganic phosphate in a hydrolysis reaction or transfers the phosphate to an acceptor such as ethanolamine or Tris (Dayan et al., (1964) Biochim. Biophys. Acta 81:620-623; Wilson et al., (1964) J. Biol. Chem. 239:4182-4185; all of which are herein incorporated by reference).

The importance of AP in clinical in vitro diagnostics and molecular biology renders it a popular subject for scientific studies and commercial development (Ferley (1971) p. 417-447, In P. D. Boyer (ed.), The Enzymes, vol. IV, Academic Press, NY; McComb et al. (ed.), (1979) Alkaline Phosphatase, Plenum Press, NY; Vallee et al., (1993) Biochemistry 32:6493-6500; all of which are herein incorporated by reference). AP and horseradish peroxidase are two major diagnostic enzymes with a world market of $15 million each (West, (1996) In T. Godfrey and S. West (ed.), Industrial Enzymology, Stockton Press, New York p. 61-68; herein incorporated by reference). During the last two decades, AP was widely used in enzyme-linked immunosorbent assay (ELISA) systems (Manson (ed.), (1992) Immunochemical Protocols, Humana Press, Totowa, N.J.; herein incorporated by reference) and non-isotopic probing, blotting, and sequencing systems (Jablonski et al., (1986) Nucleic Acids Res 14:6115-28; herein incorporated by reference).

AP has been purified and characterized from a variety of bacterial, fungal, alga, invertebrate, and vertebrate species (McComb et al., supra). However, the primary AP used commercially is calf intestine AP (CIAP) due to its high specific activity. Its usefulness, however, is limited by its inherently low thermostability and shelf life. This enzyme also has been purified from mesophiles and thermophiles. However, in general, these enzymes are unstable at room temperature or when heated, even when obtained from a thermophile. For example, a relatively unstable AP was characterized from a thermophilic Thermus species (Hartog et al., (1992) Int. J. Biochem., 24:1657-1660; herein incorporated by reference).

What is needed, therefore, is an economical and readily available thermostable AP, ideally from a hyperthermophilic organism, for use in clinical medicine and molecular biology.

### SUMMARY OF THE INVENTION

The present invention relates to compositions and methods for providing a recombinant thermostable Thermotoga (T.) neapolitana alkaline phosphatase (AP) enzyme. More particularly, the invention relates to engineering Escherichia (E.) coli with T. neapolitana alkaline phosphatase (phoA) gene expression vectors for providing an inducible system for thermostable enzyme production, wherein the expressed enzyme is readily soluble with a high degree of activity. These methods provide for producing commercial quantities of a thermostable AP enzyme.

In some embodiments, the invention provides an expression vector comprising a nucleic acid at least 78% identical to SEQ ID NO:03 encoding an alkaline phosphatase polypeptide operably linked to an inducible promoter. In other embodiments, nucleic acids are at least 78%, 80%, 85%, 90%, 95%, 98%, 99% (or more) identical to SEQ ID NO:03. In other embodiments, said nucleic acid is selected from the group consisting of SEQ ID NO:01, SEQ ID NO:03, SEQ ID NO:05, and SEQ ID NO:07. In other embodiments, the nucleic acid sequence further comprises a signal sequence SEQ ID NO: 10. In other embodiments, said alkaline phosphatase polypeptide comprises an amino acid sequence at least 79% identical to SEQ ID NO:04. Accordingly, in some embodiments, the polypeptide is at least 79%, 80%, 85%, 90%, 95%, 98%, 99% (or more) identical to SEQ ID NO:04. In still other embodiments, the nucleic acid sequence further comprises a sequence encoding a signal peptide identical to SEQ ID NO:09. In other embodiments, the nucleic acid sequence further comprises a sequence encoding an amino acid sequence at least 87% identical to

(SEQ ID NO:16)

VNVGWTTTSHSGVPVPIYAFGPGAENFTGFLDNTDIP.

Accordingly, in some embodiments, the amino acid sequence is at least 87%, 90%, 95%, 98%, 99% (or more) identical to SEQ ID NO:16. In other embodiments, said polypeptide comprises a C-terminal 6×His tag. The present invention is not limited to any particular type of polypeptide tag, indeed a variety of tags are contemplated including but not limited to C-terminal tags such as 6×His tag and green fluorescent protein. In some embodiments the nucleic acid sequence derives from a thermophilic bacterium. The present invention is not limited to any particular type of thermophilic bacterium for providing a nucleic acid sequence of the present inventions. Indeed, the use of a variety of thermophilic bacteria is contemplated including but not limited to hyperthermophilic bacterium. In some embodiments the nucleic acid derives from a Thermotoga species. In some embodiments the Thermotoga species is a Thermotoga neapolitana. In other embodiments, said expression vector further comprises a nucleic acid for increasing polypeptide production. In other embodiments, said expression vector further comprises a nucleic acid encoding a protein for increasing extracellular export of said polypeptide. In other embodiments, said protein for increasing polypeptide production is a chaperone protein. In other embodiments, said expression vector further comprises a nucleic acid selected from the group consisting of SEQ ID NOs: 75, 78, 79, 82, 84, and 86. The present invention is not limited to any particular type of promoter. Indeed, the use of a variety of promoters is contemplated including but not limited to a prokaryotic promoter, an exogenous promoter, and an inducible promoter. In other embodiments, said expression vector further comprises an inducible promoter selected from the group consisting of isopropyl-β-D thiogalactopyranosid inducible promoters.

In some embodiments, the invention provides a composition comprising a heterologous nucleic acid, wherein said nucleic acid is at least 78% identical to SEQ ID NO:03. In other embodiments the nucleic acid is at least 78%, 80%, 85%, 90%, 95%, 98%, 99% (or more) identical to SEQ ID NO:03. In other embodiments, said nucleic acid is selected from the group consisting of SEQ ID NO:01, SEQ ID NO:03, SEQ ID NO:05, and SEQ ID NO:07. In other embodiments the nucleic acid is selected from the group consisting of SEQ ID NO:01, SEQ ID NO:03, SEQ ID NO:05, and SEQ ID NO:07. In other embodiments the nucleic acid encodes a polypeptide at least 79% identical to SEQ ID NO:04. In other embodiments the nucleic acid encodes a polypeptide capable of alkaline phosphatase activity. In other embodiments alkaline phosphatase polypeptide demonstrates a specific activity of at least 1,500 U/mg at room temperature. In other embodiments the alkaline phosphatase polypeptide demonstrates a specific activity of at least 3,000 U/mg at room temperature. In other embodiments the alkaline phosphatase polypeptide demonstrates a specific activity of at least 10,000 U/mg at room temperature. In other embodiments the composition further comprises an expression vector. In other embodiments the composition further comprises an Escherichia coli host cell. The present invention is not limited to any particular type of host microorganism. Indeed, the use of a variety of host microorganisms is contemplated. In some embodiments, the microorganism is a bacterium. In other embodiments the microorganism is an Escherichia coli. In other embodiments the E. coli is a lysogenic strain of Escherichia coli In other embodiments the microorganism is a strain of E. coli. In other embodiments the Escherichia coli/is a BL21 strain.

In some embodiments, the invention provides an alkaline phosphatase (AP) polypeptide at least 79% identical to SEQ ID NO:04. Accordingly, in some embodiments, the alkaline phosphatase polypeptide is at least 79%, 80%, 85%, 90%, 95%, 98%, 99% (or more) identical to SEQ ID NO:04. In still other embodiments, the polypeptide sequence further comprises a sequence encoding a signal peptide identical to SEQ ID NO:09. In other embodiments, the polypeptide sequence further comprises a sequence at least 87% identical to

(SEQ ID NO:16)

VNVGWTTTSHSGVPVPIYAFGPGAENFTGFLDNTDIP.

Accordingly, in some embodiments, the amino acid sequence is at least 87%, 90%, 95%, 98%, 99% (or more) identical to SEQ ID NO:16. In other embodiments, said polypeptide comprises a C-terminal 6×His tag. In some embodiments the alkaline phosphatase polypeptide derives from a thermophilic bacterium. The present invention is not limited to any particular type of thermophilic bacterium for providing an alkaline phosphatase polypeptide of the present inventions. Indeed, the use of a variety of thermophilic bacteria is contemplated including but not limited to hyperthermophilic bacterium. In some embodiments the alkaline phosphatase polypeptide derives from a Thermotoga species. In other embodiments the Thermotoga species is a Thermotoga neapolitana. In some embodiments, said polypeptide comprises a C-terminal 6×His tag. In some embodiments the polypeptide is capable of alkaline phosphatase activity. In other embodiments the alkaline phosphatase polypeptide demonstrates a specific activity of at least 1,500 U/mg at room temperature. In other embodiments the alkaline phosphatase polypeptide demonstrates a specific activity of at least 3,000 U/mg at room temperature. In other embodiments the alkaline phosphatase polypeptide demonstrates a specific activity of at least 10,000 U/mg at room temperature.

In some embodiments, the invention provides a composition comprising an alkaline phosphatase (AP) polypeptide at least 79% identical to SEQ ID NO:04. Accordingly, in some embodiments, the alkaline phosphatase polypeptide is at least 79%, 80%, 85%, 90%, 95%, 98%, 99% (or more) identical to SEQ ID NO:04. In still other embodiments, the polypeptide sequence further comprises a sequence encoding a signal peptide identical to SEQ ID NO:09. In other embodiments, the polypeptide sequence further comprises a sequence at least 87% identical to

(SEQ ID NO:16)

VNVGWTTTSHSGVPVPIYAFGPGAENFTGFLDNTDIP.

Accordingly, in some embodiments, the amino acid sequence is at least 87%, 90%, 95%, 98%, 99% (or more) identical to SEQ ID NO:16. In other embodiments, said polypeptide comprises a C-terminal 6×His tag.

In some embodiments, the invention provides a method for using a thermostable alkaline phosphatase of the present inventions, comprising, a) providing, i) an alkaline phosphatase (AP) polypeptide at least 79% identical to SEQ ID NO:04, and ii) a substrate, and b) adding said alkaline phosphatase polypeptide to said substrate for providing a detectable product. In some embodiments, the product is detectable by eye. It is not intended that the present invention be limited by the type of detectable product. Indeed, in some embodiments a variety of detectable products may be used so long as the amount is in proportion to the amount of alkaline phosphatase polypeptide in the sample.

In some embodiments, the invention provides an alkaline phosphatase kit, comprising an alkaline phosphatase (AP) polypeptide at least 79% identical to SEQ ID NO:04. In some embodiments, the kit further comprises a substrate for providing a detectable product. In some embodiments, the kit further comprises instructions for use of said alkaline phosphatase (AP) polypeptide.

In some embodiments, the invention provides a method for providing commercial quantities of alkaline phosphatase of the present inventions, comprising, a) providing, i) a microorganism comprising an expression vector, wherein said expression vector comprises a nucleic acid at least 78% identical to SEQ ID NO:03, operably linked to an inducible promoter; ii) an inducing agent for said inducible promoter; ii) culture media for said microorganism; and b) contacting said microorganism with an inducing agent for expressing a commercial quantity of alkaline phosphatase polypeptide in said culture media. In other embodiments, the nucleic acid is at least 78%, 80%, 85%, 90%, 95%, 98%, 99% (or more) identical to SEQ ID NO:03. In other embodiments the method provides a commercial quantity of alkaline phosphatase at least 10 mg of purified enzyme per liter of culture media. In other embodiments the method provides a commercial quantity of alkaline phosphatase at least 15 mg of purified enzyme per liter of culture media. In other embodiments the method provides more than 15 mg of purified enzyme per liter of culture media. In other embodiments the alkaline phosphatase said nucleic acid encodes the polypeptide is at least 79% identical to SEQ ID NO:4. Accordingly, in some embodiments, the polypeptide is at least 79%, 80%, 85%, 87%, 90%, 95%, 98%, 99% (or more) identical to SEQ ID NO:4. In other embodiments the method provides a polypeptide comprising a C-terminal 6×His tag. The present invention is not limited to any particular type of promoter. Indeed, the use of a variety of promoters is contemplated. In other embodiments, the nucleic acid molecule is operably linked to an exogenous promoter. In some embodiments, the promoter is a prokaryotic promoter. In other embodiments, said expression vector further comprises an inducible promoter selected from the group consisting of isopropyl-β-D thiogalactopyranosid inducible promoters. The present invention is not limited to any particular type of inducing agent. Indeed, the use of a variety of inducing agents is contemplated. In some embodiments, the inducing agent is isopropyl-β-D thiogalactopyranosid. The present invention is not limited to any particular type of host microorganism. Indeed, the use of a variety of host microorganisms is contemplated. In some embodiments, the microorganism is a bacterium. In other embodiments the microorganism is an E. coli. In other embodiments the E. coli is a lysogenic strain of E. coli. In other embodiments the microorganism is a strain of E. coli. In other embodiments the E. coli is a BL21 strain. In other embodiments the expression vector further comprises a nucleic acid for enhancing polypeptide production by the host cell. In other embodiments the expression vector further comprises a nucleic acid encoding a protein for enhancing polypeptide production by the host cell. The present invention is not limited to any particular type of nucleic acid or protein for enhancing polypeptide production by the host cell. Indeed, the use of a variety of nucleic acids or proteins is contemplated. In other embodiments the nucleic acid for enhancing polypeptide production by the host cell is selected from the group consisting of a chaperone polypeptide, a chaperonin polypeptide, a polypeptide-export polypeptide, a polypeptide translocase polypeptide, a Sec export polypeptide, a Sec-independent translocase polypeptide, and a twin-argninine leader-binding polypeptide. In other embodiments the expression vector further comprises a nucleic acid for enhancing polypeptide secretion into said culture media. In other embodiments the expression vector further comprises a nucleic acid encoding a protein for enhancing polypeptide secretion into said culture media. The present invention is not limited to any particular type of nucleic acid or protein for enhancing polypeptide secretion into said culture media. In other embodiments the nucleic acid for enhancing polypeptide secretion is selected from the group consisting of a chaperone polypeptide, a chaperonin polypeptide, a polypeptide-export polypeptide, a polypeptide translocase polypeptide, a Sec export polypeptide, a Sec-independent translocase polypeptide, and a twin-argninine leader-binding polypeptide. In other embodiments the secretion enhancing nucleic acid is selected from the group consisting of SEQ ID NOs:75, 78, 79, 82, 84, and 86.

### DESCRIPTION OF THE FIGURES

FIG. 1 shows an exemplary sequence of A) and B). A) Sequenced T. neapolitana phoA gene (SEQ ID NO:01) lined up with its deduced amino acid sequence (SEQ ID NO:02). Ribosome binding sites are italicized and underlined. A 37-residue internal peptide identified by protein sequencing is underlined (SEQ ID NO:16). B) cloned T. neapolitana phoA gene fragment (SEQ ID NO:03) and protein sequence (SEQ ID NO:04) as cloned and expressed in pET24a(+).

FIG. 2 shows exemplary sequences of A) T. neapolitana AP nucleic acids and amino acids of the present inventions SEQ ID NOs:01-12.

FIG. 3 shows an exemplary alignment of AP proteins from E. coli (Ecol) (SEQ ID NO:13), B. subtilis (Bsub) (SEQ ID NO:14), T. neapolitana (Tnea) (SEQ ID NO:08), and bovine intestine (Bint) (SEQ ID NO:15). Active site conserved residues are in red/boxed. Active site non-conserved residues are in blue/BOLD.

FIG. 4 shows exemplary AP sequences, SEQ ID NOs:39-74.

FIG. 5 shows exemplary sequences for use in embodiments for enhancing protein production and/or secretion of T. neapolitana AP from E. coli: SEQ ID NOs:75-86.

FIG. 6 shows an exemplary plasmid map for pET24a(+) used in the present inventions.

### DEFINITIONS

To facilitate an understanding of the present invention, a number of terms and phrases as used herein are defined below:

The use of the article “a” or “an” is intended to include one or more.

As used herein, terms defined in the singular are intended to include those terms defined in the plural and vice versa.

The terms “bacteria” and “bacterium” refer to all prokaryotic organisms, including those within all of the phyla in the Kingdom Procaryotae. It is intended that the terms encompass all microorganisms considered to be bacteria, for example, Pseudomonas sp. including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms that are gram negative or gram positive. “Gram negative” and “gram positive” refer to staining patterns with the Gram-staining process, which is well known in the art. (See, e.g., Finegold and Martin, Diagnostic Microbiology, 6th Ed., CV Mosby St. Louis, pp. 13-15 [1982]; herein incorporated by reference). “Gram positive bacteria” are bacteria that retain the primary dye used in the Gram stain, causing the stained cells to appear dark blue to purple under the microscope. “Gram negative bacteria” do not retain the primary dye used in the Gram stain, but are stained by the counterstain. Thus, gram negative bacteria appear red.

As used herein, the term “microorganism” refers to any species or type of microorganism, including but not limited to, bacteria, yeast, archaea, fungi, protozoans, mycoplasma, and parasitic organisms.

The terms “eukaryotic” and “eukaryote” are used in their broadest sense. The terms include, but are not limited to, any organisms containing membrane-bound nuclei and membrane-bound organelles. Examples of eukaryotes include but are not limited to animals, yeast, alga, diatoms, and fungi.

The terms “prokaryote” and “prokaryotic” are used in their broadest sense. The terms include, but are not limited to, any organisms without a distinct nucleus. Examples of prokaryotes include but are not limited to bacteria, blue-green algae, archaebacteria, actinomycetes, and mycoplasma.

The term “gene” encompasses the coding regions of a structural gene and includes sequences located adjacent to the coding region on both the 5′ and 3′ ends for a distance of about 1 kb on either end such that the gene corresponds to the length of the full-length messenger RNA (mRNA) transcript. The sequences that are located 5′ of the coding region and that are present on the mRNA are referred to as 5′ non-translated sequences. The sequences that are located 3′ or downstream of the coding region and that are present on the mRNA are referred to as 3′ non-translated sequences. The term “gene” encompasses both cDNA and genomic forms of a gene. A genomic form or clone of a gene contains the coding regions termed “exons” or “expressed regions” or “expressed sequences” interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the mature mRNA. The mRNA functions during translation to specify the sequence or order of amino acids in a nascent polypeptide.

In addition to containing introns, genomic forms of a gene may also include sequences located on both the 5′ and 3′ ends of the sequences that are present on the mRNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage and polyadenylation.

The terms “allele” and “alleles” refer to each version of a gene for a same locus that has more than one sequence. For example, there are multiple alleles for eye color at the same locus.

The terms “nucleic acid sequence,” “nucleotide sequence of interest,” or “nucleic acid sequence of interest” refer to any nucleotide sequence (e.g., RNA or DNA), the manipulation of which may be deemed desirable for any reason (e.g., treat disease, confer improved qualities, etc.), by one of ordinary skill in the art. Such nucleotide sequences include, but are not limited to, coding sequences of structural genes (e.g., reporter genes, selection marker genes, oncogenes, disease resistance genes, growth factors, etc.), and non-coding regulatory sequences that do not encode an RNA or protein product (e.g., promoter sequence, polyadenylation sequence, termination sequence, enhancer sequence, etc.).

The term “oligonucleotide” refers to a molecule comprised of two or more deoxyribonucleotides or ribonucleotides, preferably more than three, and usually more than ten. The exact size will depend on many factors, including the ultimate function or use of the oligonucleotide. The oligonucleotide may be generated in any manner, including chemical synthesis, DNA replication, reverse transcription, or a combination thereof.

The term “polynucleotide” refers to a molecule comprised of several deoxyribonucleotides or ribonucleotides, and is used interchangeably with oligonucleotide. Typically, oligonucleotide refers to shorter lengths, and polynucleotide refers to longer lengths, of nucleic acid sequences.

The term “an oligonucleotide (or polypeptide) having a nucleotide sequence encoding a gene” or “a nucleic acid sequence encoding” a specified polypeptide refers to a nucleic acid sequence comprising the coding region of a gene or in other words the nucleic acid sequence that encodes a gene product. The coding region may be present in a cDNA, genomic DNA, or RNA form. When present in a DNA form, the oligonucleotide may be single-stranded (i.e., the sense strand) or double-stranded. Suitable control elements such as enhancers/promoters, splice junctions, polyadenylation signals, etc., may be placed in close proximity to the gene coding region if needed to permit proper transcription initiation and/or correct processing of the primary RNA transcript. Alternatively, the coding region utilized in the expression vectors of the present invention may contain endogenous enhancers, exogenous promoters, splice junctions, intervening sequences, polyadenylation signals, etc., or a combination of endogenous and exogenous control elements.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” when only a subset of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

When made in reference to a nucleic acid molecule, the term “recombinant” refers to a nucleic acid molecule that is comprised of segments of nucleic acid joined together by means of molecular biological techniques. When made in reference to a protein or a polypeptide, the term “recombinant” refers to a protein molecule that is expressed using a recombinant nucleic acid molecule.

The terms “protein,” “polypeptide,” “peptide,” “encoded product,” and “amino acid sequence” are used interchangeably to refer to compounds comprising amino acids joined via peptide bonds. A “protein” encoded by a gene is not limited to the amino acid sequence encoded by the gene, but includes post-translational modifications of the protein. Where the term “amino acid sequence” is recited herein to refer to an amino acid sequence of a protein molecule, the term “amino acid sequence” and like terms, such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein. The deduced amino acid sequence from a coding nucleic acid sequence includes sequences that are derived from the deduced amino acid sequence and modified by post-translational processing, where modifications include but are not limited to glycosylations, hydroxylations, and phosphorylations, as well as amino acid deletions, substitutions, and additions. Thus, an amino acid sequence comprising a deduced amino acid sequence is understood to include post-translational modifications of the encoded and deduced amino acid sequence. The term “X” may represent any amino acid.

As used herein, the terms “polymerase chain reaction” and “PCR” refer to the method described in U.S. Pat. Nos. 4,683,195, 4,889,818, and 4,683,202, all of which are hereby incorporated by reference. These patents describe methods for increasing the concentration of a segment of a target or heterologous sequence in a mixture of genomic DNA without cloning or purification. This process for amplifying the target sequence consists of adding a large excess of two oligonucleotide primers to the DNA mixture containing the desired target sequence, followed by a precise sequence of thermal cycling in the presence of a DNA polymerase (e.g., Taq). The primers are either complementary or identical to their respective strands of the double stranded target sequence. To effect amplification, the mixture is denatured and the primers then annealed to their complementary sequences within the target molecule. Following annealing, the primers are extended with a polymerase to form a new pair of complementary strands. The steps of denaturation, primer annealing, and polymerase extension can be repeated many times (i.e., denaturation, annealing, and extension constitute one “cycle”; there can be numerous “cycles”) to obtain a high concentration of an amplified segment of the desired target sequence. The length of the amplified segment of the desired target sequence is determined by the relative positions of the primers with respect to each other, thus this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA or mRNA to a level detectable by several different methodologies (i.e., agarose gels stained with ethidium bromide, hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

As used herein, the terms “PCR product” and “PCR fragment” refer to the resultant mixture of compounds after two or more cycles of the PCR steps of denaturation, annealing, and extension are complete. These terms encompass the case where there has been amplification of one or more segments of one or more target sequences.

The term “reverse-transcriptase-PCR” or “RT-PCR” refers to a type of “PCR” and “polymerase chain reaction” where the starting material is mRNA. The starting mRNA is enzymatically converted to complementary DNA or “cDNA” using a reverse transcriptase enzyme. The cDNA is then used as a “template” for a “PCR” reaction.

The term “primer” refers to an oligonucleotide (whether occurring naturally as in a purified restriction digest or produced synthetically) that can act as a point of initiation of synthesis when placed under conditions in which synthesis of a primer extension product which is complementary to a nucleic acid strand is induced (i.e., in the presence of nucleotides and an inducing agent such as DNA polymerase and at a suitable temperature and pH). The primer is preferably single-stranded for maximum efficiency in amplification, but may alternatively be double-stranded. If double-stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact length of the primer will depend on many factors, including temperature, source of primer, and the use of the method.

The term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “isolated” when used in relation to a nucleic acid or polypeptide, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be used to express a protein, the oligonucleotide will contain at a minimum the sense or coding strand (i.e., the oligonucleotide may be single-stranded), but may contain both the sense and anti-sense strands (i.e., the oligonucleotide may be double-stranded).

The term “purified” refers to molecules, either nucleic or amino acid sequences that are removed from their natural environment. An “isolated nucleic acid sequence” is therefore a purified nucleic acid sequence. “Substantially purified” molecules are at least 60% free, preferably at least 75% free, and more preferably at least 90% free from other components with which they are naturally associated.

As used herein, the terms “purified” and “to purify” or “purifying” also refer to the removal of contaminants from a sample. The removal of contaminating proteins results in an increase in the percent of polypeptide of interest in the sample. In another example, recombinant polypeptides are expressed in bacterial, yeast, bacteria, or mammalian host cells and the polypeptides are purified by the removal of host cell proteins; the percent of recombinant polypeptides is thereby increased in the sample.

The term “host cell” refers to any cell capable of replicating and/or transcribing and/or translating a heterologous gene, such as a heterologous gene encoded by an expression vector. Thus, a “host cell” refers to any eukaryotic or prokaryotic cell (e.g., bacterial cells such as E. coli, other bacterial cells, yeast cells, animal cells, and plant cells), whether located in vitro or in vivo.

The terms “transfection” and “transfecting” refer to the introduction of foreign DNA into cells. Transfection may be accomplished by a variety of means known to the art including mixing competent bacteria with plasmid DNA followed by incubation, electroporation, microinjection, phage infection, and the like.

The term “competent” refers to the ability of a host cell to take up exogenous DNA and thereby be transformed (e.g., competent bacteria).

The term “transgene” refers to a foreign gene that is placed into an organism by the process of transfection.

The terms “foreign gene” or “heterologous gene” refers to any nucleic acid (e.g., gene sequence) that is introduced into the genome of an organism by experimental manipulations and may include gene sequences found in that organism so long as the introduced gene does not reside in the same location, as does the naturally occurring gene. For example, a heterologous gene includes a gene from one species introduced into another species. A heterologous gene also includes a gene native to an organism that has been altered in some way (e.g., mutated, added in multiple copies, linked to non-native regulatory sequences, etc).

The terms “transformants” and “transformed cells” include the primary transformed cell and cultures derived from a transfected cell without regard to the number of transfers following transfection. Progeny may not be precisely identical in DNA content, due to deliberate or inadvertent mutations. Mutant progeny that have the same functionality as screened for in the originally transformed cell are included in the definition of transformants.

The term “selectable marker” refers to a gene that encodes an enzyme or protein having an activity that confers resistance to an antibiotic or drug to the cell in which the selectable marker is expressed, or which confers expression of a trait that can be detected (e.g., color of colonies, luminescence or fluorescence, or antibiotic resistance, or growth factor). Selectable markers may be “positive” or “negative.” Examples of positive selectable markers include the neomycin phosphotrasferase (NPTII) gene that confers resistance to G418 and to kanamycin, and the bacterial hygromycin phosphotransferase gene (hyg), which confers resistance to the antibiotic hygromycin. Negative selectable markers encode an enzymatic activity whose expression is cytotoxic to the cell when grown in an appropriate selective medium. For example, the HSV-tk gene is commonly used as a negative selectable marker. Expression of the HSV-tk gene in cells grown in the presence of gancyclovir or acyclovir is cytotoxic; thus, growth of cells in selective medium containing gancyclovir or acyclovir selects against cells able to express a functional HSV TK enzyme.

The term “reporter gene” refers to a gene encoding a protein that may be assayed. Examples of reporter genes include, but are not limited to, luciferase (See, e.g., deWet et al., Mol. Cell. Biol. 7:725 (1987) and U.S. Pat. Nos. 6,074,859; 5,976,796; 5,674,713; and 5,618,682; all of which are incorporated herein by reference), green fluorescent protein (e.g., GenBank Accession Number U43284; a number of GFP variants are commercially available from ClonTech Laboratories, Palo Alto, Calif.), chloramphenicol acetyltransferase, β-galactosidase, alkaline phosphatase, and horseradish peroxidase. The results of the assay are a signal produced by the reporter gene; the signal is detectable when it is above background.

The term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and may further refer to the process, where applicable, of converting genetic information of mRNA into protein, through mRNA “translation.” Gene expression can be regulated at many stages in the process. “Up-regulation” or “activation” refers to regulation that increases the production of gene expression products (i.e., RNA or protein), while “down-regulation” or “repression” refers to regulation that decreases production.

Molecules (e.g., transcription factors) involved in up-regulation and down-regulation are often called “activators” and “repressors,” respectively.

The terms “in operable combination”, “in operable order” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “regulatory element” refers to a genetic element that controls some aspect of the expression of nucleic acid sequences. For example, a promoter is a regulatory element that facilitates the initiation of transcription of an operably linked coding region. Other regulatory elements are splicing signals, enhancers, polyadenylation signals, termination signals, et cetera.

Transcriptional control signals or transcriptional regulatory elements in eukaryotes comprise “promoter” and “enhancer” elements. Promoters and enhancers consist of short arrays of DNA sequences that interact specifically with cellular proteins involved in transcription (Maniatis, et al., Science 236:1237, 1987; herein incorporated by reference). Promoter and enhancer elements have been isolated from a variety of eukaryotic sources including genes in yeast, insect, and mammalian cells. Promoter and enhancer elements have also been isolated from viruses, and analogous control elements, such as promoters, are also found in prokaryotes. The selection of a particular promoter and enhancer depends on the cell type used to express the protein of interest. Some eukaryotic promoters and enhancers have a broad host range while others are functional in a limited subset of cell types (for review, see Voss, et al., (1986) Trends Biochem. Sci., 11:287; and Maniatis, et al., supra 1987; all of which are herein incorporated by reference).

The terms “promoter element,” “promoter,” or “promoter sequence” as used herein, refer to a DNA sequence that is located at the 5′ end (i.e. precedes) the protein coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

Promoters may be constitutive or inducible. The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue.

In contrast, a “regulatable” or “inducible” promoter is a promoter able to direct a level of transcription of an operably linked nuclei acid sequence, in the presence of an “inducing agent” or “inducing stimulus” (e.g., IPTG, heat shock, chemicals, light, etc.), that is different from the level of transcription of the operably linked nucleic acid sequence in the absence of the stimulus.

The enhancer and/or promoter may be “endogenous” or “exogenous” or “heterologous.” An “endogenous” enhancer or promoter is one that is naturally linked with a given gene in the genome. An “exogenous” or “heterologous” enhancer or promoter is one that is placed in juxtaposition to a gene by means of genetic manipulation (i.e., molecular biological techniques) such that transcription of the gene is directed by the linked enhancer or promoter. For example, an endogenous promoter in operable combination with a first gene can be isolated, removed, and placed in operable combination with a second gene, thereby making it a “heterologous promoter” in operable combination with the second gene. A variety of such combinations are contemplated (e.g., the first and second genes can be from the same species, or from different species.

Efficient expression of recombinant DNA sequences in eukaryotic cells requires expression of signals directing the efficient termination and polyadenylation of the resulting transcript. Transcription termination signals are generally found downstream of the polyadenylation signal and are a few hundred nucleotides in length. The term “poly(A) site” or “poly(A) sequence” as used herein denotes a DNA sequence which directs both the termination and polyadenylation of the nascent RNA transcript. Efficient polyadenylation of the recombinant transcript is desirable, as transcripts lacking a poly(A) tail are unstable and are rapidly degraded. The poly(A) signal utilized in an expression vector may be “heterologous” or “endogenous.” An endogenous poly(A) signal is one that is found naturally at the 3′ end of the coding region of a given gene in the genome. A heterologous poly(A) signal is one which has been isolated from one gene and positioned 3′ to another gene. A commonly used heterologous poly(A) signal is the SV40 poly(A) signal. The SV40 poly(A) signal is contained on a 237 bp BamHI/BclI restriction fragment and directs both termination and polyadenylation (Sambrook, supra, at 16.6-16.7).

The term “vector” refers to nucleic acid molecules that transfer DNA segment(s) from one cell to another. The term “vehicle” is sometimes used interchangeably with “vector.”

The terms “expression vector” or “expression cassette” refer to a recombinant DNA molecule containing a desired coding sequence, such as a phoA coding region or coding regions for proteins for enhancing protein secretion, such as proteins for enhancing production and/or extracellular transport of AP proteins, and appropriate nucleic acid sequences necessary for the expression of the operably linked coding sequence in a particular host organism, such as a promoter sequence. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The term “sample” is used in its broadest sense. In one sense it can refer to a bacterial cell or culture medium or secreted product. In another sense, it is meant to include a protein or culture obtained from any source, as well as biological and environmental samples. Biological samples may be obtained from bacteria or animals (including humans) and encompass cells, growth medium, secreted products, fluids, solids, tissues, and gases. These examples are not to be construed as limiting the sample types applicable to the present invention.

The term “wild-type” when made in reference to a nucleic acid sequence or amino acid sequence refers to a sequence that has the characteristics of sequence s isolated from a naturally occurring source. The term “wild-type” when made in reference to a sequence also refers to a gene and a gene product, that have the characteristics of a gene and a gene product isolated from a naturally occurring organism. A wild-type sequence is that which is most frequently observed in a population and is thus arbitrarily designated the “normal” or “wild-type” form of the sequence and genes found within that organism.

In contrast, the term “modified” or “mutant” when made in reference to a sequence refers to a sequence comprising a gene or to a gene product, respectively, that displays modifications in sequence and/or functional properties (i.e., altered characteristics) when compared to the wild-type gene or gene product expressed in wild-type organism. It is noted that naturally-occurring mutants can be isolated; these are identified by the fact that they have altered characteristics when compared to the wild-type sequence and the expressed wild-type gene or gene product.

As used herein, “substrate” as in a biocatalytic reaction refers to a chemical entity whose conversion to a product or products such as a “detectable product,” is catalysed by one or several enzymes, examples of a substrate for use with the alkaline phosphatases of the present inventions include but are not limited to reduction of tetrazolium salts, such as those described herein, or the production of colored diazo compounds, such as 5-bromo-4-chloro-3-indolyl phosphate.

As used herein, “detectable product” refers to any product detectable by any means known in the art, such as by eye, film, phosphorous screen, and the like.

As used herein, the terms “alkaline phosphatase,” “phosphate-monoester phosphohydrolase,” “alkaline optimum,” “alkaline phosphomonoesterase,” “phosphomonoesterase,” “glycerophosphatase,” alkaline phosphohydrolase,” “alkaline phenyl phosphatase,” “orthophosphoric-monoester phosphohydrolase,” refer to an enzyme described under IUBMB Enzyme Nomenclature as EC 3.1.3.1.

As used herein, the term “secretory protein” refers to a protein intended for export from a cell, such as exporting an alkaline phosphatase from a bacterium.

As used herein, the terms “chaperone” and “molecular chaperone” refer to a protein whose function is to assist other proteins in achieving proper folding, or unfolding, for altering exportation of a protein from a cell. A chaperone may include but is not limited to a “high temperature protein” or “htp” and “heat shock protein” or Hsp,” “chaperonin,” SecB,” “Syc,” and the like.

As used herein, the term “chaperonin” refers to a protein or a protein complex that assists in the folding of nascent, non-native polypeptides into their native, functional state, and for altering exportation of protein or a protein complex from a cell. Examples include molecular chaperones or Group I chaperonins or Group II chaperonins.

As used herein, the term “Group I chaperonin” refers to a chaperonin found in prokaryotes, for example, a “GroEL/GroES” complex in E. coli.

As used herein, the term “GroEL” refers to a protein chaperone that is required for the proper folding of many proteins in prokaryotes, in addition to a “GroES” referred to in general as a “cochaperone protein” or specifically as a “chaperonin 60” and “chaperonin 10,” respectively to GroEL/GroES.

As used herein, the term “dnaK” refers to a gene encoding a “Hsp70” chaperone protein (approximately 70 kDa) in E. coli that is sometimes regulated by a “dnaJ” that encodes a “Hsp40.”

As used herein, the term “HtpG” in reference to a protein refers to an E. coli chaperone protein in E. coli related to “Hsp90.”

As used herein, the term “Clp” refers to a family of E. coli proteins that target and unfold tagged and misfolded proteins, for example, ClpA and ClpX are related to Hsp100 chaperones and associate with such proteins as a serine protease ClpP.

As used herein, the terms “secretion-enhancing nucleic acid” and “secretion-enhancing protein” refers to a nucleic acid sequence and its encoded protein involved in increasing the amount of secretory protein that is exported from a cell, e.g. a protein for increasing extracellular AP protein when compared to the amount of extracellular protein measured in the absence of the secretion-enhancing nucleic acid or protein for increasing secretion of an AP protein.

As used herein, the terms “protein for enhancing protein production” or “protein for enhancing protein secretion” or “protein for increasing protein production” or “protein for increasing protein secretion” refers to a protein or polypeptide sequence that when present increases the amount of desired or target protein produced over the amount produced under identical conditions when the protein is not present. For the purposes of the present inventions, the term “increasing the amount of protein produced” refers to both increasing the amount of protein within a host cell and increasing the amount of protein secreted by the host cell into the cell medium.

As used herein, the term “enhancing” is equivalent to “increasing” and “enhanced” is equivalent to “increased” that for the purpose of the present inventions refers to a value of a sample that is at least 2× or more greater than a value for a comparison sample.

### DESCRIPTION OF THE INVENTION

The present invention relates to compositions and methods for providing a recombinant thermostable Thermotoga neapolitana alkaline phosphatase (AP) enzyme. More particularly, the invention relates to engineering Escherichia coli with Thermotoga neapolitana alkaline phosphatase gene (phoA) expression vectors for providing an inducible system for thermostable AP enzyme production, wherein the expressed enzyme is readily soluble with a high degree of activity. These methods provide for producing commercial quantities of a thermostable AP enzyme.

Currently, commercially available mammalian AP is isolated from calf intestine, (Invitrogen, Calbiochem, New England Biolabs, etc.) and an invertebrate AP is isolated from cold-living northern shrimp (SAP) (P. borealis). These enzymes are inactivated by heating at 65° C. for 15 min. (see, Roche Diagnostics Corporation R&D). Recombinant AP is commercially available from mouse (i.e. an alpl gene (rmALPL, expressed with a N-terminal signal peptide and a C-terminal 6H is tag in a murine myeloma cell line, NS0; R&D Systems) and human (i.e. an ALPL gene (rhALPL) expressed with a N-terminal signal peptide and a C-terminal 6H is tag in a murine myeloma cell line, NS0; R&D Systems). Further, an extremophile Antarctic AP is commercially available (i.e. produced from an E. coli strain that carries the TAB5 AP gene (New England Biolabs) however this AP is 100% heat inactivated in 5 minutes at 65° Celsius. Despite their rapid inactivation when exposed to higher temperatures (around 65° Celsius) and their well-known characteristic of becoming inactive when stored or used for short time-periods at room temperature, these AP enzymes are widely used in molecular biology and other applications because of their relative high specific activity. Specific activity of commercially available mammalian AP refers to ≧1500 units/mg, where one unit is defined as the amount of enzyme that will hydrolyze 1.0 μmol of p-nitrophenyl phosphate (pNPP) per minute at 25° C. at pH 9.6. However, the usefulness of calf intestine AP is limited by its inherently low thermostability and short shelf life, for example, calf intestine IP quickly looses activity within 30 minutes at room temperature.

Other APs were purified and characterized from a variety of bacterial, fungal, algal, invertebrate, and vertebrate species (McComb et al., (ed.), (1979) Alkaline Phosphatase, Plenum Press, NY; herein incorporated by reference) including APs from mesophiles, thermophiles (for example, Hartog et al., (1992) Int. J. Biochem. 24:1657-1660; herein incorporated by reference), an extreme thermophile (Kim et al., (1997) J. Biochem. Mol. Biol. 30:262-268; herein incorporated by reference), a psychrophilic bacterium (as in bacteria thriving at relatively low temperatures, (for example, bacteria isolated in the Anarctic; and in Rina et al., (2000) Eur. J. Biochem. 267, 1230-1238; herein incorporated by reference) and hyperthermophiles (for examples, Lee, et al. (1996) Biosci Biotechnol Biochem. 60(5):840-6; Wojciechowski et al., 2002, Protein Sci. 11(4):903-11; Dong and Zeikus, Enzyme Microb Technol. 1997 October; 21(5):335-40; all of which are herein incorporated by reference). A relatively unstable AP was characterized from a thermophilic Thermus species (Hartog et al., (1992) Int. J. Biochem. 24:1657-1660; herein incorporated by reference) while a thermostable AP was isolated from Thermus caldophilus GK24 (Kim et al., (1997) J. Biochem. Mol. Biol. 30:262-268; herein incorporated by reference) and from a thermophilic species, Thermus thermophilus, (see, for example, U.S. Pat. No. 5,633,138; herein incorporated by reference).

The E. coli AP enzyme is a homodimer, of which each dimer is composed of 449-residue subunits (Bradshaw et al., (1981) Proc. Natl. Acad. Sci. USA 78:3473-3477; Chang et al., (1986) Gene 44:121-125; all of which are herein incorporated by reference). Each monomer folds into an alpha/beta structure, with 10 β-strands making up the central β-sheet, flanked by 15 helices. Each monomer contains two Zn²⁺ and one Mg²⁺ cations located near the active site and interacting with phosphate. Three main functional differences can be distinguished between eubacterial (E. coli) and mammalian APs: (1) eubacterial APs are considerably more thermostable than their mammalian counterparts; (2) mammalian APs are 20 to 30 times more active; and (3) mammalian APs are optimally active at higher pHs. Mammalian (i.e., calf intestine) AP's higher catalytic activity was explained by the presence of two histidines in this enzyme at positions corresponding to Asp 153 and Lys328 in the E. coli enzyme (Dealwis et al., (1995) Protein Eng. 8:865-871; Murphy et al., (1994) Mol. Microbiol. 12:351-357; Murphy et al., (1995) J. Mol. Biol. 253:604-617; all of which are herein incorporated by reference). Thus although E. coli AP was more stable than mammalian (calf) AP, it demonstrated a significantly lower specific activity.

In contrast, a thermostable AP was purified from a hyperthermophilic bacterium, T. neapolitana (Dong et al., (1997) Enzyme Microbial Technol. 21:335-340; herein incorporated by reference). The enzyme was a homodimer composed of two 45 kDa subunits. The isolated enzyme was optimally active at 85° C. and pH 9.9. Under these conditions, it displayed 30% higher activity than calf intestine AP did on pNPP. T. neapolitana AP (TNAP) demonstrated a half-life of 238 min at 90° C. (compared to 60 min. at 65° C. for a commercial mammalian AP enzyme) and was stable at room temperature over a broad pH range. The inventors contemplated that the unique features of thermostability and high specific activity would make this enzyme very attractive for thermostability studies and for industrial and commercial applications. Further, thermostability, including retaining stability when stored at room temperature, and a high specific activity of AP from a native T. neapolitana AP, renders this enzyme ideally suitable for molecular biological applications requiring these characteristics, see, for example, T. neapolitana alkaline phosphatase protein isolation and characteristics (see, for example, Dong and Zeikus, Enzyme Microb Technol. (1997) 21(5):335-40; and U.S. Pat. No. 5,980,890; all of which are herein incorporated by reference).

Therefore, the inventors contemplated that an AP enzyme from hyperthernophiles, in particular TNAP, is an attractive alternative to calf intestine AP for diagnostic applications in the pharmaceutical and food industries, such as for use in immunoassays, provided they are active at moderate temperatures (i.e., under conditions compatible with the biological activity and stability of the other reagents involved in the assay) (see, Vieille and Zeikus, 2001, Microbiol Mol Biol, Rev 65:1-43; herein incorporated by reference).

It is not intended that the present invention be limited by the particular thermophilic AP sequence. For example, AP may derive from such thermophiles and hyperthernophiles from Order “Thermotogales” (see, Reysenbach 2002, Int J Syst Evol Microbiol 2002 52: 685-690; herein incorporated by reference) or Thermales (see, Rainey and Da Costa 2002, Int. J. Syst. Evol. Microbiol., 2002, 52:7-76; herein incorporated by reference; Genus Thermus, Meiothermus, Marinithermus, Oceanithermus, Vulcanithermus, and the like; Order Deinococcales (see, Rainey et al. 1997, Int. J. Syst. Bacteriol., 1997, 47:510-514; herein incorporated by reference), Genus Deinococcus, Thermus, Thermales, and the like; and thermophilic Bacillus; Aquificales; Archaeoglobales; Thermococcales, and the like. In a preferred embodiment, the inventors contemplate an AP derived from hyperthermophilic genera within the Bacteria (Aquifex, Thermotoga), Euryarchaeota (Archaeoglobus, Ferroglobus, Thermococcus, Pyrococcus, Palaeococcus), and Crenarchaeota (Pyrodictium, Staphylothermus, Thermodiscus). The phenotypes of the well-characterized isolates include aerobes and anaerobes, chemolithoautotrophs and heterotrophs, acidophiles, and neutrophiles. Specifically, examples of bacterial sources of AP sequences include T. neapolitana DSM 4359, Thermotoga lettingae TMOT, Thermotoga petrophila RKU-1, Thermotoga maritima MSB8, Thermus flavus, Thermus thermophilus HB8, Thermus thermophilus HB27, and Deinococcus-Thermus, such as Deinococcus geothermalis DSM11300, and the like.

The Examples presented herein, describe a cloned and sequenced T. neapolitana phoA gene, expressed as a catalytically active protein, including expressed with a C-terminal 6×His tag, purified, and characterized as a recombinant enzyme of the present inventions.

The following sections describe Compositions and Methods for providing commercial quantities of recombinant T. neapolitana AP: I) Native and Recombinant AP production in Escherichia coli, II) T. neapolitana AP production in Escherichia (E) coli, and III) Methods for increasing T. neapolitana AP production in Escherichia (E) coli. Further provided are methods of use for the AP enzymes of the present inventions.

I. Native and Recombinant AP Production in Escherichia (E) coli.

In Escherichia coli, native AP is located in the periplasmic space, also known as the periplasm, located between the plasma membrane and the outer membrane as in other gram-negative bacteria. AP is involved in recovering phosphate from esters when free inorganic phosphate is depleted (Schwartz et al., (1961) Proc. Natl. Acad. Sci. USA 47: 1996-2005; herein incorporated by reference). Escherichia coli was shown to express heterologous E. coli AP from expression vectors comprising such AP genes. One example of an E. coli expressing a recombinant E. coli AP is shown in International publication No. WO1994001531; herein incorporated by reference.

However, when sequences encoding various types of heterologous APs, also referred to as recombinant APs, were transformed into E. coli, these APs, similar to other expressed heterologous proteins, form inclusion bodies in the cytoplasm, inhibiting production of soluble and active enzyme, and thus yielding amounts too small for commercial purposes. Several attempts were made to increase the production and secretion of AP produced in bacteria, in particular, mutating E. coli AP, see, for example, International publication No. WO1994001531; herein incorporated by reference, or attaching signal sequences and further attaching secretion enhancing sequences from heterologous proteins, see, for example, International publication No. WO/1989/010971; herein incorporated by reference. Further, low yields were measured when a hyperthermophilic Thermotoga maritima phoA gene was expressed in a T7 RNA polymerase system (pET23a, Novagen®) in E. coli, (Wojciechowski, et al., 2002, Protein Sci. 11(4):903-11; herein incorporated by reference).

Therefore, there is a need for compositions and methods of providing commercially viable levels of recombinant AP that remains thermostable while catalytically active.

II. T. neapolitana AP (TNAP) Production in Escherichia (E) coli.

Unexpectedly, the DNA construct of the present invention, lacking a coding region for a TNAP signal sequence, expressed in E. coli cells of the present invention, yielded approximately 15 mg of catalytically active enzyme from one liter of the bacterial culture (see, Example VII). This quantity is increased, at least 3× greater, than published yields of 2-5 mg of pure protein earlier produced per liter culture, see, Wojciechowski, supra. Even lower yields were obtained when the T. maritima phoA gene was expressed with a putative signal sequence in the E. coli EK1597 strain using the IMPACT-CN system from New England Biolabs and a pET24 derivative pEK453. This expressed AP showed a specific activity of 2 U/mg where activity was increased on addition of Co(II) and Mg(II) and exposure to heat to 88 U/mg at 25° C., and under certain conditions attained a maximal activity of 289 U/mg, see, Wojciechowski, supra. Thus compositions and methods of the present invention provide commercial quantities of AP enzyme. Additionally, the inventors contemplate compositions and methods of further increasing the yield of catalytically active AP, such that the amount of AP produced by a host cell provides an economically viable source of AP for commercial production.

The following sections describe aspects of the present invention with embodiments based upon actual experiments, of which certain exemplary information is shown in Examples below, or described as embodiments contemplated by the inventors.

A. TNAP Genes, Coding Sequences, and Polypeptides.

The present invention provides hyperthermophilic bacterially derived AP genes and proteins, including their homologs, orthologs, paralogs, variants, and mutants. The present invention is not limited to the use of any particular homolog or variant or mutant of the TNAP gene or the TNAP protein. Indeed, in some embodiments a variety of TNAP genes or TNAP proteins, variants and mutants thereof, may be used so long as they retain at least some of the activity of the corresponding wild-type protein. Functional variants can be screened for by expressing the variant in an appropriate vector (described in more detail below) in a bacterial cell and analyzing the enzyme's economical viability (e.g. stability at room temperature or at 4° C., specific activity, amount of protein produced per liter of bacterial culture, specific activity per liter of bacterial culture, etc.).

A preferred AP gene sequence of the present invention is a DNA sequence that encodes a prokaryotic AP that when expressed in bacteria, such as E. coli, using methods of the present inventions, demonstrates a specific activity equal to or greater than 1500 U/mg, at least 3000 U/mg, and further at least 7000 U/mg to 10,000 U/mg or higher at 80° Celsius. TNAP activity is preferably measured by following the release of p-nitrophenol from pNPP in 0.2 M Tris-HCl (pH 10.4) at 80° Celsius. One enzyme activity unit (U) represents the hydrolysis of 1 μmole of substrate per min under these standard assay conditions. For comparison, commercially available calf intestinal AP), (CIP) is reported to demonstrate a specific activity of at least 10,000 units/ml, alternatively, a specific activity of 3,500 units/mg, where one “Unit” or “U” refers to the amount of enzyme that hydrolyzes 1 μmol of pNPP to p-nitrophenol in a total reaction volume of 1 ml in 1 minute at 37° C. (Mossner, et al., (1980) Hoppe Seyler's Z. Physiol. Chem. 361, 543-549; herein incorporated by reference) of which the reaction takes place in a standard reaction buffer, for example, 1 M diethanolamine-HCl (pH 9.8) with 0.5 mM MgCl₂ and 10 mM pNPP (see, New England BioLabs Technical Bulletin #M0290S (Jul. 31, 2006).

1. T. neapolitana AP (TNAP) Genes.

The present invention provides hyperthermophilic bacterially derived phoA genes, including their homologs, orthologs, paralogs, variants, and mutants. In some embodiments, isolated nucleic acid sequences comprising phoA coding regions are provided, for example, SEQ ID NO: 01. These sequences include nucleic acid sequences comprising a phoA coding region for a TNAP protein, for example, SEQ ID NO: 02. The present invention further provides nucleic acid sequences having a portion of the coding sequence for a mature TNAP protein (or a portion of a TNAP protein), for example, SEQ ID NO: 07, for a mature TNAP protein, for example, SEQ ID NO: 08. In some embodiments, isolated nucleic acid sequences comprising cloned TNAP coding regions are provided, for example, SEQ ID NO: 03. In some embodiments, the TNAP coding region is at least 78% identical to SEQ ID NO:03.

Any given gene may have none, one or many allelic forms. Common mutational changes that give rise to alleles are generally ascribed to deletions, insertions, or substitutions of nucleic acids. Each of these types of changes may occur alone, or in combination with the others, and at the rate of one or more times in a given sequence. In some embodiments of the present invention, mutations in these genes, which alter expression of the genes, result in increased enzyme production. In some embodiments of the present invention, mutations in these genes, which alter location of the expressed protein, result in increased enzyme production. In some embodiments of the present invention, mutations in these genes, which alter activity of the expressed protein, result in increased specific activity of enzyme.

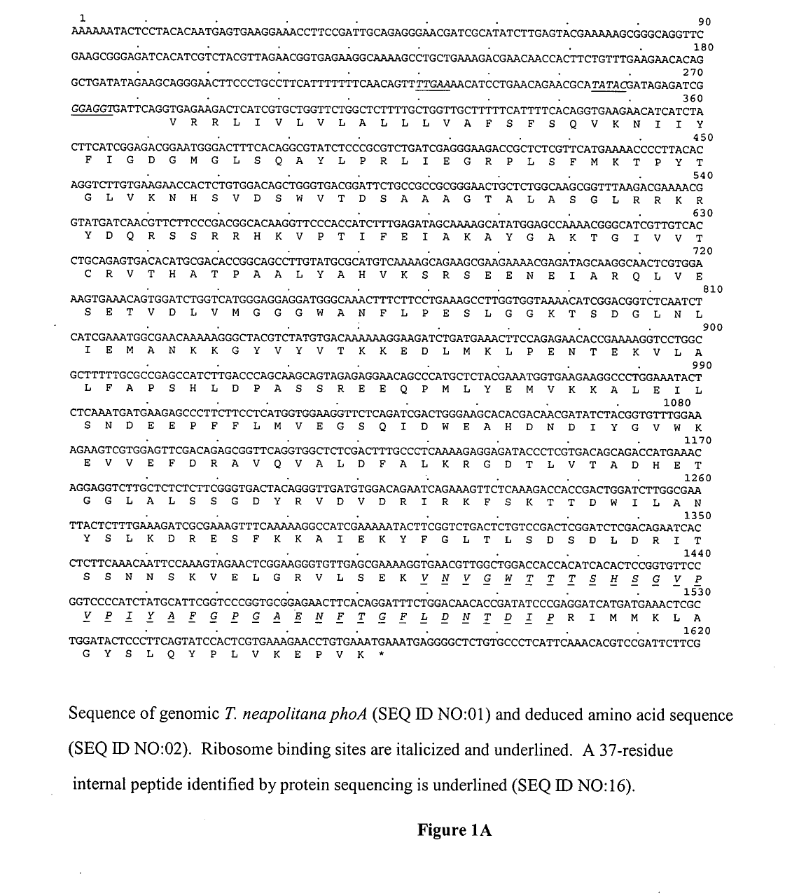

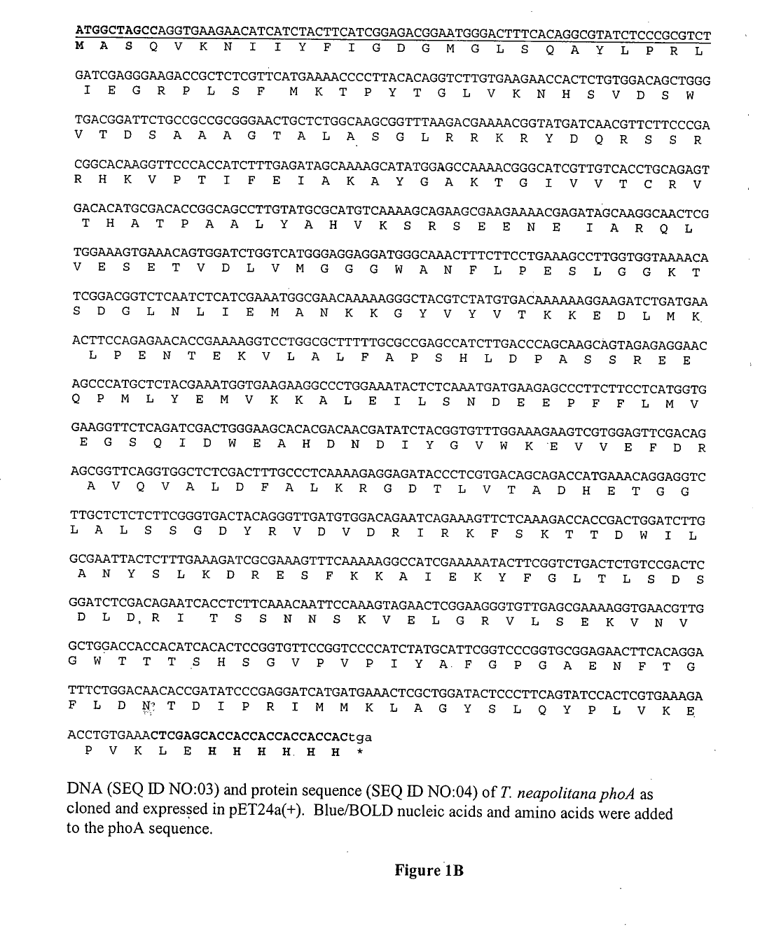

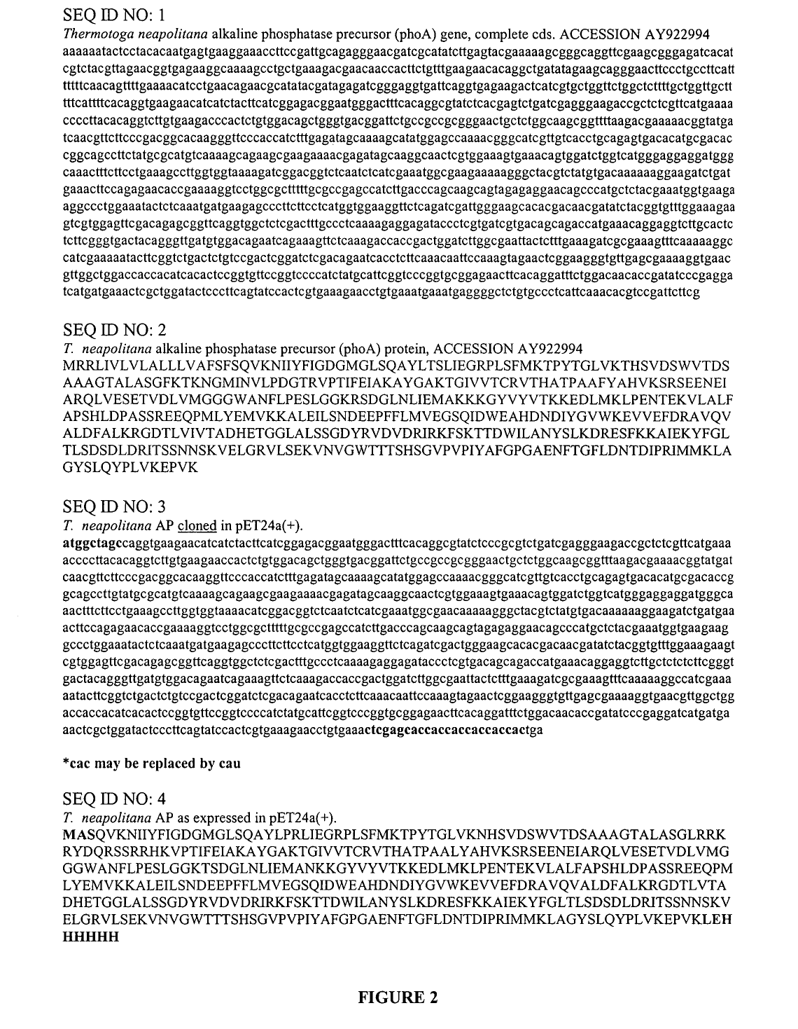

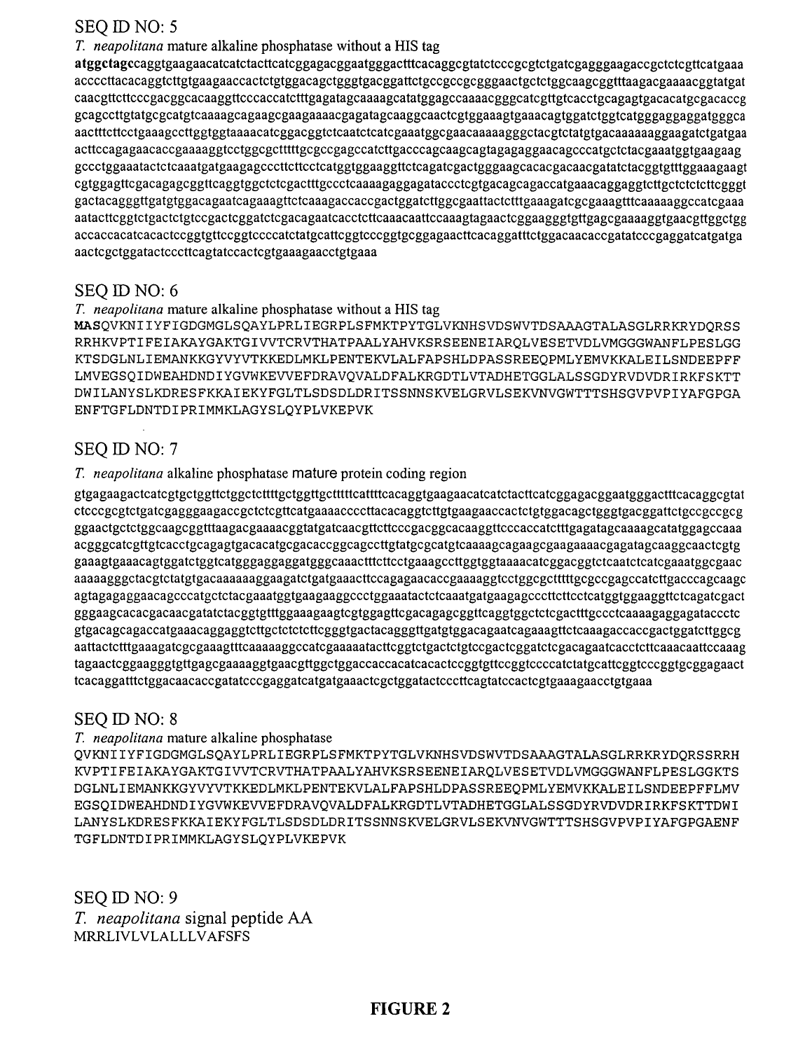

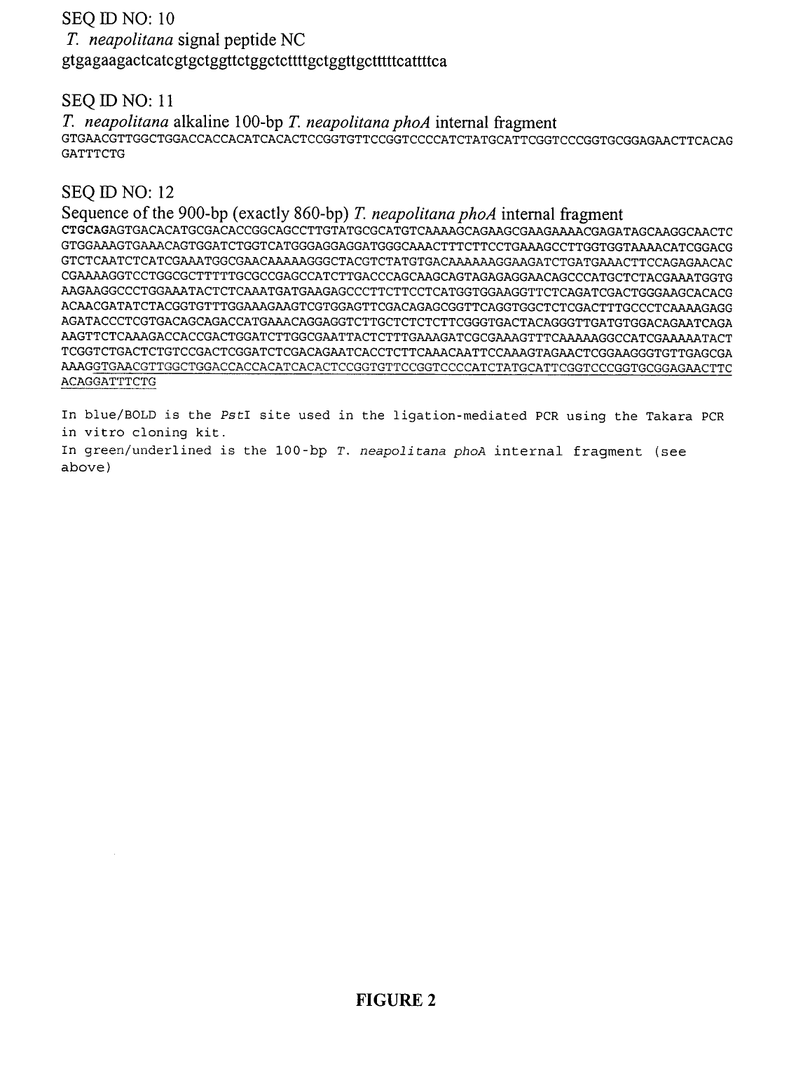

Mutational changes in alleles also include rearrangements, insertions, deletions, or substitutions in upstream regulatory regions. In some embodiments of the present invention, mutations in these genes, which alter ribosomal binding sites, result in increased enzyme expression. In some embodiments, the inventors contemplate altering identified ribosomal binding sites, see, FIG. 1A.

2. Thermotoga neapolitana AP (TNAP) Polypeptides.

The present invention provides isolated TNAP and/or TNAP-like polypeptides, as well as variants, homologs, mutants or fusion proteins thereof, as described above. In some embodiments of the present invention, the polypeptide is a naturally purified product, while in other embodiments it is a product of chemical synthetic procedures, and in still other embodiments it is produced by recombinant techniques using a prokaryotic or eukaryotic host (e.g., by bacterial, yeast, insect, and mammalian cells in culture). In some embodiments, depending upon the host employed in a recombinant production procedure, the polypeptide of the present invention is glycosylated or non-glycosylated. In other embodiments, the polypeptides of the invention also include initial MAS amino acid residues at the amino terminal end. In other embodiments, the polypeptides of the invention also include LE amino acid residues at the carboxy terminal end. In other embodiments, the polypeptides of the invention further include HHHHHH (6×H) amino acid residues at the carboxy terminal end.

a. Purification of TNAP Polypeptides.

The present invention provides purified AP polypeptides or contemplates purified AP polypeptide variants, homologs, mutants or fusion proteins thereof, as described herein. The present invention also provides methods for recovering and purifying TNAP, with or without tags, such as polyhistidine tags, from recombinant cells. In other embodiments of the present invention, cells are typically harvested by centrifugation, disrupted by physical or chemical means, and the resulting crude extract retained for further purification. In still other embodiments of the present invention, microbial cells employed in expression of proteins can be disrupted by any convenient method, including freeze-thaw cycles, sonication, passage through a French pressure cell, mechanical disruption, or use of cell lysing agents. The present invention also provides methods for recovering and purifying TNAP, with or without tags, such as polyhistidine tags, from recombinant cell extracts or culture medium including, but not limited to, ammonium sulfate or ethanol precipitation, acid extraction, chromatofocusing, anion or cation exchange chromatography, phosphocellulose chromatography, hydrophobic interaction chromatography, affinity chromatography, hydroxylapatite chromatography, and lectin chromatography. In some embodiments of the present invention, AP polypeptides purified from recombinant organisms as described herein are provided. In particular embodiments, AP polypeptides were purified from bacterial cell medium of bacterial cells transformed with phoA coding regions, as described herein.

b. Purification of TNAP Polypeptides with Polyhistidine Tags.

The present invention contemplates methods for recovering and purifying TNAP, with tags, such as polyhistidine tags, from recombinant cell cultures. Whether purifying proteins from E. coli, yeast or other eukaryotic cells, the first step is disruption of cells and extraction of the relevant protein fraction. Harsh mechanical or enzymatic treatments affect the heterologous protein's structural integrity and activity. Protein extraction reagents such as BugBuster® (see, User Protocol TB245 Rev. E 0304, Novagen; herein incorporated by reference) are rapid, low-cost alternatives to mechanical methods such as French Press or sonication for releasing expressed heterologous protein in preparation for purification or other applications. Benzonase, His•Bind® or other chromatography matrices (Novagen) are preferably utilized for purification of proteins that have histidine tags. Heterologous proteins that contain highly charged domains may also associate with other cellular components (e.g., basic proteins may bind to DNA). In these cases, the heterologous protein may partition with cellular debris; in theory, they may be dissociated by adding salt to the lysis buffer or digesting the nucleic acid with a nuclease such as Benzonase® Nuclease (see Novagen User Protocol TB261, Novagen; herein incorporated by reference).

The PopCulture® His•Mag™ Purification Kit is designed for purification of His•Tag® fusion proteins directly from E. coli cultures without harvesting cells. The procedure combines PopCulture total culture extraction with magnetic affinity purification using His•Mag Agarose Beads (User Protocol TB054 Rev. F 0106, Novagen; herein incorporated by reference).

B. Engineered Constructs.

1. Expression Vectors.

The present invention also provides expression vectors for expressing TNAP polypeptides. In some embodiments of the present invention, vectors include, but are not limited to chromosomal, nonchromosomal and synthetic DNA sequences (e.g., derivatives of bacterial plasmids, phage DNA, bacteria tumor sequences, T-DNA sequences, baculovirus, yeast plasmids, vectors derived from combinations of plasmids and phage DNA). It is contemplated that any vector may be used as long as it is replicable and viable in the host cell.

In particular, some embodiments of the present invention provide recombinant constructs comprising one or more of the nucleic acid sequences as broadly described herein, (e.g., SEQ ID NOs: 01, 03, 06 and 07). In some embodiments of the present invention, the constructs comprise a vector, such as a bacterial or eukaryotic vector, or viral vector, into which a nucleic acid sequence of the invention has been inserted, in a forward or reverse orientation. In some embodiments of the present invention, the nucleic acid sequences are inserted as a single copy per vector. In some embodiments of the present invention, the nucleic acid sequences are inserted as two or more copies per vector. In some embodiments of the present invention, the nucleic acid sequences are inserted as six or more copies per vector. In preferred embodiments of the present invention, the appropriate nucleic acid sequence is inserted into the vector using any of a variety of procedures. In general, the nucleic acid sequence is inserted into an appropriate restriction endonuclease site(s) by procedures known in the art.

Large numbers of suitable vectors are known to those of skill in the art, and are commercially available. Such vectors for incorporation into host cells include, but are not limited to, the following vectors and their derivatives: 1) Prokaryotic and other host cells—pBI221, pBI121 (Clonetech), pYeDP60, pQE70, pQE60, pQE-9 (Qiagen), pBS, pD10, phagescript, psiX174, pbluescript SK, pBSKS, pNH8A, pNH16a, pNH18A, pNH46A (Stratagene); ptrc99a, pKK223-3, pKK233-3, pDR540, pRIT5 (Pharmacia); pBI2113Not, pBI2113, pBI101, pBI121, pGA482, pGAH, PBIG, and 2) Eukaryotic and other host cells—pHISi-1, pMLBART, Agrobacterium tumefaciens strain GV3101, pSV2CAT, pOG44, PXT1, pSG (Stratagene); pSVK3, pBPV, pMSG, and pSVL (Pharmacia); pLGV23Neo, pNCAT, and pMON200. Any other plasmid or vector may be used as long as they are replicable and viable in the host.

In some preferred embodiments of the present invention, bacteria expression vectors comprise an origin of replication, a suitable promoter and enhancer, and also any necessary ribosome binding sites, polyadenylation sites, splice donor and acceptor sites, transcriptional termination sequences, and 5′ flanking nontranscribed sequences for expression in bacteria. In other embodiments, DNA sequences derived from the SV40 splice, and polyadenylation sites may be used to provide the required nontranscribed genetic elements.

In other embodiments of the present invention, recombinant expression vectors include origins of replication and selectable markers permitting transformation of the host cell (e.g., dihydrofolate reductase or neomycin resistance for eukaryotic cell culture, or tetracycline, kanamycin, or ampicillin resistance in E. coli).

In other embodiments, the expression vector also contains a ribosome binding site for translation initiation and a transcription terminator. In still other embodiments of the present invention, the vector may also include appropriate sequences for amplifying expression.

In one embodiment, the coding part of the phoA gene is ligated into an expression vector and transferred to a matching suitable E. coli strain for protein expression under optimized conditions for protein production. In a preferred embodiment, the production of TNAP protein is at commercially viable levels. In a further embodiment, commercially viable levels of TNAP are enzymes with high specific activity.

a. Expression Vectors for Providing HIS Tags.

The present invention provides expression vectors for providing polyhistidine tagged AP polypeptides. In one embodiment, the coding region of the phoA gene is ligated into a vector that provides nucleic acid sequences for expressing a protein tag. One example is a pET24a vector that adds a hexahistidine (6×HIS), which in turn adds a 6×HIS tag to the C-terminus of an expressed protein, such as a TNAP. Another example is a pQE-30 vector which adds a hexahistidine (6×HIS) tag to the N-terminus of an expressed protein.

b. Expression Vectors for Periplasmic Localization of a Heterologous Protein.

The present invention contemplates expression vectors for targeting AP production to the periplasmic space of a bacterium. Previous efforts to provide commercially viable amounts of a protein, including compositions and methods for avoiding inclusion body formation, included attempts to provide active soluble proteins expressed by vectors that express proteins directly into the periplasm. Advantages of this directed expression include providing a more favorable environment for folding and disulfide bond formation (Raina, et al., (1997) Annu Rev Microbiol., 51:179-202; Rietsch, et al., (1996) Proc. Natl. Acad. Sci. U.S.A. 93(23):13048-53; and Sone., et al., (1997) J. Biol. Chem., 272: 10349-10352; all of which are herein incorporated by reference). Thus in one embodiment, the inventors contemplate heterologous expression and/or transport of TNAP directly into the periplasm of host E. coli. In one embodiment, the inventors contemplate expression vectors comprising nucleic acid targeting sequences, such as periplasmic targeting sequences, for targeting heterologous polypeptides to the periplasm.

c. Expression Vectors for Co-Expression Systems.

The present invention contemplates co-expression of proteins for increasing AP production. Co-expression of multiple copies of heterologous genes in E. coli demonstrated enhanced yield, solubility, and activity of certain heterologous proteins that either make up part of a multi-protein complex, including dimers, or whose expression is increased with co-expression of a protein for increasing desired/target protein production. Further, the inventors contemplate co-expression of a heterologous gene in E. Coli together with adaptor molecules for increasing production of a desired protein, such as co-expressing a chaperone protein with a TNAP protein. It is well known that co-expression greatly facilitated the production of multi-subunit complexes and biochemical pathways and the characterization of protein-protein interactions, among other applications (Novy, R., et al. 2002, in Novations 15 by Novagen; herein incorporated by reference).

Co-expression of multiple copies of heterologous genes or multiple heterologous genes in E. coli can be achieved by either cloning and expressing two or more open reading frames (ORFs) in a single vector or by transforming cells with two or more plasmids with compatible replicons and different drug resistance genes, for example, using two or more plasmids such as Duet Co-expression Vectors (Novagen), described below. The following T7 promoter-based vectors with adaptor molecules are contemplated for use in the present invention for co-expression of multiple desired DNA sequences in E. coli host cells.

In one embodiment, a Duet Co-expression system of up to five vectors, each of which is capable of coexpressing two heterologous proteins or, when transformed with one another, or with other pET vectors, coexpressing up to eight proteins in one cell in E. coli when using appropriate host strains (see, User Protocol TB340, Novagen, herein incorporated by reference) increases TNAP production. In one embodiment, a pETcoco™ System of two vectors that are compatible with many expression vectors and have the added benefit of allowing control over the number of copies present per cell for cloning and expression purposes, (see, User Protocol TB333, Novagen, herein incorporated by reference) increases TNAP production. In one embodiment, a LIC Duet™ Adaptor is used to convert a pET, pRSF, or pCDF Ek/LIC-prepared plasmid into a co-expression vector (see, User Protocol TB384, Novagen, herein incorporated by reference) to increase TNAP production. These adaptors are designed to facilitate the simultaneous cloning of two ORFs into one plasmid and their subsequent coepxression in E. coli. Five adaptors are available, four of which encode fusion tags that aid in purification and/or may enhance solubility of the heterologous protein. The fifth is a “mini” adaptor for minimal vector-encoded fusion sequences.

2. Promoters.

Proteins can be expressed in eukaryotic cells, yeast, bacteria, or other cells under the control of appropriate promoters. The present invention provides a nucleic acid sequence in the expression vector, operatively linked to an appropriate expression control sequence(s) (promoter), to direct mRNA synthesis. Promoters useful in the present invention include, but are not limited to, T7 and T3 promoters, the E. coli lac and trp promoters, the phage lambda P_{L} and P_{R} promoters and the LTR promoter of SV40, cytomegalovirus (CMV) immediate early, herpes simplex virus (HSV) thymidine kinase, and mouse metallothionein-I promoters and other promoters known to control expression of genes in prokaryotic or eukaryotic cells or their viruses.

In some embodiments of the present invention, DNA encoding the polypeptides of the present invention were expressed using bacterial promoters. Bacterial promoters can be inducible, constitutive, leaky, and transient, such as an inducible promoter, e.g. isopropyl-β-D thiogalactopyranoside (IPTG)-inducible promoters found in a variety of commercially available prokaryotic expression vectors.

a. TNAP Promoters.

The present invention provides a TNAP promoter in a TNAP expression vector, for example, pTNAP1. In one embodiment, a promoter of the present invention is a TNAP promoter, see, FIG. 1A.

b. Inducible Promoters.

The present invention provides a promoter for a TNAP gene chosen for optimal expression in a matching strain of E. coli for high levels of protein expression and to optimize protein production conditions. For example, a TNAP gene was isolated and cloned into a plasmid for expression in E. coli, wherein IPTG-inducible promoter sequences were used to induce recombinant protein (such as, for example, SEQ ID NO:04) expression.

In some embodiments of the present invention, following transformation of a suitable host strain and growth of the recombinant strain to an appropriate cell density, the selected inducible promoter is induced by appropriate means (e.g., chemical induction) and cells are cultured for an additional period for optimal production of the desired protein.

3. Host Escherichia (E) coli Strains.

The present invention provides host cells containing the constructs described herein. In some embodiments of the present invention, the host cell is a prokaryotic cell (e.g., a bacterium). In some embodiments of the present invention, the host cell is a gram-negative bacterium. An example of compositions and methods for providing a bacteria cell transgenic for phoA are provided in U.S. Pat. No. 4,375,514, herein incorporated by reference. In some embodiments of the present invention, the host cell is a gram-positive bacterium. An example of a phoA transgenic gram-positive bacterial cell and methods thereof are provided in U.S. Pat. No. 4,745,056, herein incorporated by reference. In some contemplated embodiments of the present invention, the host cell is a eukaryotic cell (e.g., a yeast). An example of a eukaryotic phoA transgenic yeast cell and methods thereof are provided in U.S. Pat. No. 6,884,602; and United States Patent Application No. 20030096341; all of which are herein incorporated by reference.

Specific examples of host cells useful to the present invention include, but are not limited to, E. coli, Streptomyces, Pseudomonas aeruginosa, Pseudomonas syringae, Pseudomonas putida, Pseudomonas fluorescens, Pseudomonas testosteroni, Serratia marcescens and Erwinia herbicola, as well as Saccharomyces cerivisiae, Schizosaccharomyces pombe, Drosophila S2 cells, Spodoptera Sf9 cells, Chinese hamster ovary (CHO) cells, COS-7 lines of monkey kidney fibroblasts, (Gluzman, Cell 23:175 (1981), herein incorporated by reference), 293T, C127, 3T3, HeLa and BHK cell lines, NT-1 (tobacco cell culture line), root cell and cultured roots in rhizosecretion (Gleba, et al., Proc Natl Acad Sci USA 96: 5973-5977 (1999); herein incorporated by reference).

Cell-free translation systems can also be employed to produce such proteins using mRNAs derived from the DNA constructs of the present invention. Appropriate cloning and expression vectors for use with prokaryotic and eukaryotic hosts are described by Sambrook, et al., Molecular Cloning: A Laboratory Manual, 2nd ed., Cold Spring Harbor Laboratory Press, New York (1989); herein incorporated by reference.

In one embodiment, the coding part of the phoA gene is ligated into a suitable expression vector and transferred to a matching suitable E. coli strain for protein expression under optimized conditions for protein production. In a preferred embodiment, the production of TNAP protein is at commercially viable levels. In a further embodiment, commercially viable levels of TNAP are enzymes with high specific activity.
III. Methods for Increasing Thermotoga neapolitana AP Production.

A. Enhancing Solubility and Folding.

Recombinant proteins expressed in E. coli are often produced as aggregates called inclusion bodies. Even when inclusion bodies are formed, some portion of the heterologous protein is usually soluble within the cell. With the high expression levels of certain expression vectors, such as the pET System used in the present invention, there may be a significant amount of soluble protein produced even when most of the heterologous protein mass is aggregated in an inclusion body. In general, conditions that decrease the rate of protein synthesis, such as low induction temperatures or growth in minimal media, tend to increase the percentage of heterologous protein in soluble form. In many applications, it is desirable to express heterologous proteins in their soluble, active form. The following sections describe contemplated compositions and methods to enhance solubility and thus production of a heterologous protein. It should be noted that solubility does not necessarily indicate that a protein is folded properly; some proteins form soluble species that are inactive. In a preferred embodiment of the present invention, methods are contemplated for increasing the production of TNAP with a high specific activity.

1. E. coli Bacteria Growth Conditions for Increasing TNAP Production.

a. Temperature.

Culturing E. coli at 37° C. causes some heterologous proteins to accumulate as inclusion bodies, while incubation at 30° C. may lead to increasing soluble, active heterologous protein (Schein, et al., (1989) Bio/Technology 7:1141-1149; herein incorporated by reference). Culturing and induction, such as IPTG induction, at 25° C. or 30° C., may increase export of heterologous proteins. In some embodiments, prolonged (e.g., overnight) induction at low temperatures (15′-20° C.) may prove optimal for the yield of soluble protein.

b. Lysis Buffer.

Partitioning of a given heterologous protein, such as TNAP, into a soluble or insoluble fraction is strongly influenced by the nature of the lysis buffer used for providing protein extracts. Proteins containing hydrophobic or membrane-associated domains may not actually be present in inclusion bodies however when using a standard lysis buffer, without a non-ionic detergent, these proteins may partition into the insoluble fraction that includes inclusion bodies. Therefore, in some embodiments, the inventors contemplate the addition of millimolar amounts of nonionic detergent, such as Triton® X-100, Saponin, TWEEN® 20 and the like, or zwitterionic detergents CHAPS (3-[(3-Cholamidopropyl)dimethylammonio]-propanesulfonic acid) and the like, to the lysis buffer for increasing the amount of soluble expressed protein.

2. Proteins of the Twin-Arginine Translocation (Tat) Pathway.

In many prokaryotic organisms, secretory proteins harboring a twin-arginine consensus motif are exported in a fully folded conformation via the twin-arginine translocation (Tat) pathway. In E. coli, Tat involves three structurally and functionally different membrane proteins TatA, TatB, and TatC (Lee, et al., Annual Review of Microbiology 60 (First posted online on Jun. 6, 2006, publication date October 2006) (Grkovic, Microbiol Mol Biol Rev. 2002, 66(4):671-701; herein incorporated by reference). Whereas the TatC protein functions in the specific recognition of substrate, TatA might be the major pore-forming subunit for aiding in exportation of proteins.

In one embodiment, the inventors contemplate decreasing inclusion body formation by compositions of AP encoding sequences further comprising tat coding regions (for example, see, Muller, Res Microbiol. (2005) 156(2):131-6. Epub 2005, January 28; herein incorporated by reference). In other attempts for increasing AP secretion, a Thermus thermophilus phoA construct was used in conjunction with the TAT translocation pathway for expressing AP in E. coli (Angelini, et al., 2001, FEBS Lett. 506(2):103-7; herein incorporated by reference).

3. Chaperone Proteins.

Chaperone proteins often increase the production of recombinant proteins in E. coli. For example, a cytoplasmic chaperone was used to increase the secretion of E. coli AP (Kononova, et al., 2001, Biochemistry (Mosc). 66(7):803-7; herein incorporated by reference). In one embodiment, the inventors contemplate increasing protein production by coexpressing AP enzyme with chaperone proteins.

4. Fusion Proteins.

In other attempts for increasing protein secretion, in particular, AP production, E. coli mutants showed increased protein production, a reported 6- to 16-fold increase, when expressing a functional AP fused to a protein, for example, a scFv-PhoA hybrid (Belin, et al., (2004) Protein Eng Des Sel. 17(5):491-500; herein incorporated by reference). Thus in one embodiment, the inventors contemplate TANP expression as a fusion protein.

5. Rare tRNA Supplementation.

Amino acids are usually encoded by more than one codon, and each organism carries its own bias in the usage of the 61 available amino acid codons. The tRNA population of an organism closely reflects the codon bias of the mRNA population of that organism. Analysis of E. coli codon usage reveals that several codons are underrepresented, typically paralleling the E. coli tRNA population. Therefore, when heterologous genes are overexpressed in E. coli, differences in codon usage can impede translation due to the demand for one or more tRNAs that may be rare or lacking in E. coli. Insufficient tRNA pools can lead to translational stalling, premature translation termination, translation frameshifting, and amino acid misincorporation, thus inhibiting heterologous protein expression.

Although the presence of a small number of rare codons often does not severely depress heterologous protein synthesis, heterologous protein expression can be very low when a gene contains clusters of and/or numerous rare E. coli codons. Excessive rare codon usage in the heterologous gene has been implicated as a cause for low-level expression (Sorensen et al., 1989 J Mol. Biol., 207(2):365-77; Zhang et al., 1991, Gene, 105(1):61-72; all of which are herein incorporated by reference) as well as truncation products. The effect appears most severe when multiple rare codons occur near the amino terminus (N-terminus) (Chen, et al., 1990, Nucleic Acids Res., 1990, 18(6):1465-73; herein incorporated by reference). A number of studies have indicated that high usage of the arginine codons AGA and AGG can have severe effects on protein yield. The impact appears to be highest when these codons are present near the N-terminus and when they appear consecutively (Brinkmann, et al., 1989, Gene, 85(1):109-14; Calderone, et al., 1996, J Mol. Biol., 262(4):407-12; Hua, et al., 1994, Biochem Mol Biol Int., 32(3):537-43; Schenk, et al., 1995, Biotechniques, 19(2):196-200; Zahn, 1996, J. Bacteriol., 178(10):2926-33 and Mol. Microbiol., 21(1):69-76; all of which are herein incorporated by reference). Several laboratories have shown that the yield of protein whose genes contain rare codons can be dramatically improved when the population of the cognate tRNA is increased within the host (Brinkmann et al., 1989, Gene, 85(1):109-14; Rosenberg, et al., 1993, J. Bacteriol. 175(3):716-22; Seidel, et al., 1992, Biochemistry, 1(9):2598-608; all of which are herein incorporated by reference). For example, the yield of human plasminogen activator was increased approximately 10-fold in a strain that carried an extra copy of the tRNA for AGG and AGA on a compatible plasmid (Brinkmann et al., 1989, Gene, 85(1):109-14; herein incorporated by reference). Increasing other rare tRNAs for AUA, CUA, CCC, or GGA has also been used to augment the yield and fidelity of heterologous proteins (Kane, 1995, Curr Opin Biotechnol., 6(5):494-500; herein incorporated by reference).

Furthermore, attempts were made to enhance AP protein production in E. coli by co-transforming plasmids, such as pSJS1240, encoding tRNA genes for arginine codons AGA and AGG and isoleucine codon AUA, which are not typically expressed at high levels in E. coli (Wojciechowski, et al., 2002, Protein Sci. 11(4):903-11; herein incorporated by reference). When plasmids encoding rare tRNA genes were co-expressed with a T. maritima phoA gene without the putative signal sequence in an IMPACT-CN system (E. coli strain ER2566, New England Biolabs) 2-5 mg of pure AP protein were obtained per liter culture (Wojciechowski, et al., 2002, Protein Sci. 11(4):903-11; herein incorporated by reference).

Thus the inventors contemplate that supplementing the expression systems of the present invention with rare tRNA codons would increase the amount of protein produced. In one embodiment, the inventors contemplate decreasing inclusion body formation by using hosts and plasmids encoding rare codons for E. coli. An example of such a product for increasing protein production would be using the E. coli Rosetta™ strain (Novagen) that supplement tRNAs rarely utilized in E. coli on a chloramphenicol resistant plasmid (pACYC backbone) compatible with pET vectors as the TNAP expression host strain. Rosetta™ strains (Novagen) are designed to enhance the expression of eukaryotic proteins by supplying codons rarely used in E. coli such as tRNAs for the codons AUA, AGG, AGA, CUA, CCC and GGA on a compatible chloramphenicol-resistant plasmid (B Brinkmann et al., 1989, Gene, 85(1):109-14; Kane, 1995, Curr Opin Biotechnol., 6(5):494-500; Kurland et al., 1996; Seidel et al., 1992; all of which are herein incorporated by reference).

### IV. Methods of Using Alkaline Phosphatases of the Present Inventions.

Alkaline phosphatases in combination with their substrates are widely used in well-known diagnostic, experimental, and industrial applications including but not limited to ELISAs (Reen, et al, (1994) Meth. Mol. Biol. 32:461; herein incorporated by reference), immunohistochemistry (Sugasawara, et al, (1984) J. Clin. Microbiol. 19:230: herein incorporated by reference), and Northern, Southern and Western blot techniques. Alkaline phosphatase—substrate reactions produce a wide variety of well-known types of reaction products, such as chromogenic, fluorogenic, chemiluminescent, and fluorescent products. Examples of well-known substrates may be obtained commercially, for example, Vector Laboratories, Burlingame, Calif., USA, and Molecular Imaging Products Company, OR, USA.

As one example, a number of histochemical chromogenic substrates for alkaline phosphatase are commercially available and give reaction products with a range of colors, such as blue (BCIP blue: 5-bromo-4-chloro-3-indolyl phosphate/NBT Alkaline Phosphatase Substrate Solution) or pink (BCIP Pink: (6-chloro-3-indoxyl phosphate, p-toluidine salt), for brightfield examination. Some of these reaction products are also fluorescent, exhibiting a wide excitation range and a broad emission peak. Other examples are substrate kits, such as a VECTOR Black Substrate Kit, a VECTOR Blue Substrate Kit, a VECTOR Red Substrate Kit (Vector Laboratories, Burlingame, Calif., USA).

The known alkaline phosphatase substrate kits which provide reagents for forming reaction product precipitates are based on either reduction of tetrazolium salts or the production of colored diazo compounds. When stored at 4° C., these kits are stable for about one year. These substrate kits (VECTOR) were developed to produce different colored precipitates which are permanently mounted in non-aqueous media. Three colors can be effectively introduced into a section to localize three antigens in different cells even using the same species of primary antibody, the same biotinylated secondary antibody and the VECTASTAIN® ABC-AP reagent. The BCIP/NBT substrate is frequently used for nitrocellulose or in situ hybridization applications. However, p-Nitro phenylphosphate substrate is the preferred substrate for enzyme immunoassays for neuronal cells.

A specific example for Brightfield Histochemistry and High-resolution Fluorescence Imaging by Confocal Laser Scanning Microscopy is Vector Blue III (Vector Laboratories, Burlingame, Calif., USA). Vector Blue III when added to an alkaline phosphatase yields a stable, strongly fluorescent reaction product with an excitation peak around 500 nm and a large Stokes shift to an emission peak at 680 nm. The reaction product is excited using a mercury lamp with a fluorescein excitation filter or an argon ion laser at 488 nm or 568 nm, and the emission detected using a long-pass filter designed for Cy-5. Thus, a single substrate is suitable for brightfield imaging of tissue sections and high-resolution analysis of subcellular detail, using a confocal laser scanning microscope, in the same specimen.

A specific example for immunology based assays using AP enzymes of the present inventions are as follows. In one embodiment, the AP enzymes of the present inventions are used for detecting antibody binding to an antigen. Antibody binding is detected by techniques known in the art. For example, in some embodiments where protein is detected in cells (e.g., body cells, such as white blood cells), antibody binding is detected using a suitable technique, including but not limited to, radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays. In other embodiments, where protein is detected in tissue samples, immunohistochemistry can be utilized for the detection of antibody binding.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include, but are not limited to, those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a diagnosis and/or prognosis based on the presence or absence of a series of proteins corresponding to cancer markers is utilized.

In other embodiments, the immunoassay described in U.S. Pat. Nos. 5,599,677 and 5,672,480, each of which is herein incorporated by reference, is utilized. In other embodiments, proteins are detected by immunohistochemistry.

Further, an example of a substrate solution is as follows. A BCIP/NBT Alkaline Phosphatase Substrate Solution—Blue substrate, is provided by a working solution of 0.02% BCIP and 0.03% NBT in 0.1M TBS, pH 9.5 (Vector Laboratories, Burlingame, Calif., USA).

Another method of use for an alkaline phosphatase enzyme of the present inventions is for molecular biology reactions, such as dephosphorylation of DNA Fragments with Alkaline Phosphatase. Such that, essentially any protein phosphatase (e.g., bacterial alkaline phosphatase [BAP], calf intestinal phosphatase [CIP], placental alkaline phosphatase, and shrimp alkaline phosphatase [SAP]) will catalyze the removal of 5′ phosphates from nucleic acid templates. Because CIP and SAP are readily inactivated, they are the most widely used phosphatases in molecular cloning. Although CIP is cheaper per unit of activity, SAP enzyme has the advantage of being readily inactivated in the absence of chelators (see, Molecular Cloning, 3rd edition, by Joseph Sambrook and David W. Russell. Cold Spring Harbor Laboratory Press, Cold Spring Harbor, N.Y., USA, 2001; herein incorporated by reference).

Therefore, the inventors contemplate using their Thermotoga neapolitana alkaline phosphatase (AP) enzymes in combination with a wide variety of substrates. Thus in one embodiment, the inventors contemplate substituting known AP enzymes with an AP enzyme of the present inventions. In one embodiment, the AP enzyme of the present inventions would be used with well-known working solutions, buffers and protocols. In one embodiment, the amount used of an AP enzyme of the present inventions would provide an equivalent specific activity to the replaced enzyme. In other embodiments, the inventors contemplate using AP enzyme of the present inventions under novel conditions, such as novel working solutions, ingredients, buffers and protocols.

The inventors further contemplate a kit comprising alkaline phosphatase (AP) enzymes of the present inventions. In some embodiments, the present invention provides kits for the detection of proteins or nucleic acids. In some embodiments, the kits contain antibodies specific for a protein, in addition to detection reagents and buffers. In other embodiments, the kits contain reagents specific for the detection of a specific protein's mRNA or cDNA (e.g., oligonucleotide probes or primers). In preferred embodiments, the kits contain the components necessary to perform an entire detection assay, including controls, directions for performing assays. Further, these kits may comprise any necessary software for analysis and presentation of results.

### EXPERIMENTAL

The following examples serve to illustrate certain embodiments and aspects of the present invention and are not to be construed as liming the scope thereof. In the experimental disclosures that follow, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); pg (picograms); L and l (liters); ml (milliliters); μl (microliters); cm (centimeters); mm (millimeters); μm (micrometers); nm (nanometers); U (units); min (minute); s and sec (second); k (kilometer); deg (degree); C (degrees Centigrade/Celsius).

Chemical sources for protein purification and assays described below; DEAE-Sepharose Sephacryl S 200 and Phenyl-Sepharose were purchased from Pharmacia Fine Chemica AB, Uppsala, Sweden. Histidyldiazobenzylpropionic acid-Agarose, pNPP, adenosine-5′-diphosphate disodium salt (ADP), adenosine-5′-triphosphate disodium salt (ATP), β-glycerol-phosphate, D-glucose-1-phosphate, D-glucose-6-phosphate, D-fructose-6-phosphate, D-fructose-1,6-diphosphate and Triton X-100 were purchased from Sigma, Chemical Co., U.S.A. Ethylenediamine tetraacetic acid disodium salt (EDTA) and alkaline phosphatase (calf intestine) were purchased from Boehringer Mannheim GmbH, Germany.

DNA manipulations. DNA manipulations (e.g., plasmid DNA purification, restriction analysis, PCR, and colony and DNA hybridization) were performed using established protocols (Sambrook et al., (1989) Molecular Cloning: A Laboratory Manual, 2nd Ed. Cold Spring Harbor Laboratory Press, Cold Spring Harbor, N.Y.; Ausubel et al., (1993) In Struhl, K. (ed.), Current Protocols in Molecular Biology. Greene Publishing and Wiley-InterScience, New York). DNA fragments were recovered from agarose gels using the Geneclean II kit (BIO 101, La Jolla, Calif.; herein incorporated by reference). Oligonucleotides used in this study (Table 2) were synthesized by the Michigan State University Macromolecular Structure Facility. Ligation-mediated PCR was performed using the PCR in vitro cloning kit (PanVera, Madison, Wis.; herein incorporated by reference), and primers S1 and S2 (Table 2). The restriction enzyme used for phoA gene analysis was PstI. To overexpress TNAP in E. coli, the T. neapolitana phoA gene was amplified using primers 3 and 4 (Table 2) and subcloned in pET24a(+) between the NdeI and XhoI sites, generating plasmid pTNAP3.

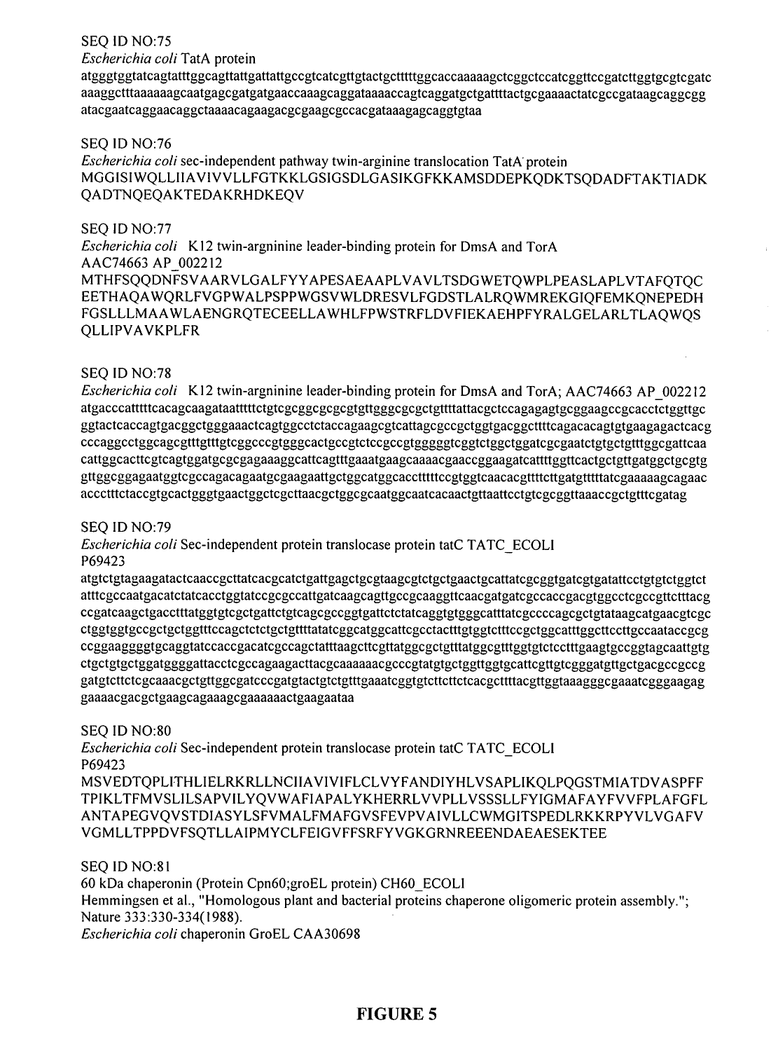

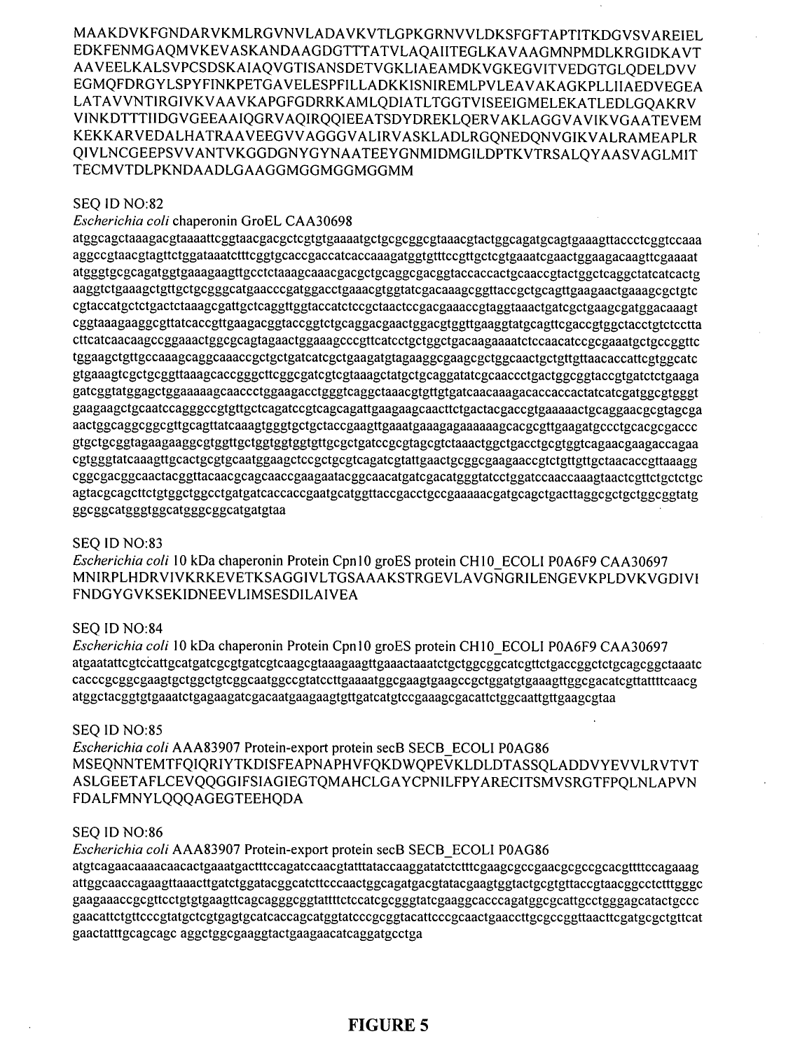

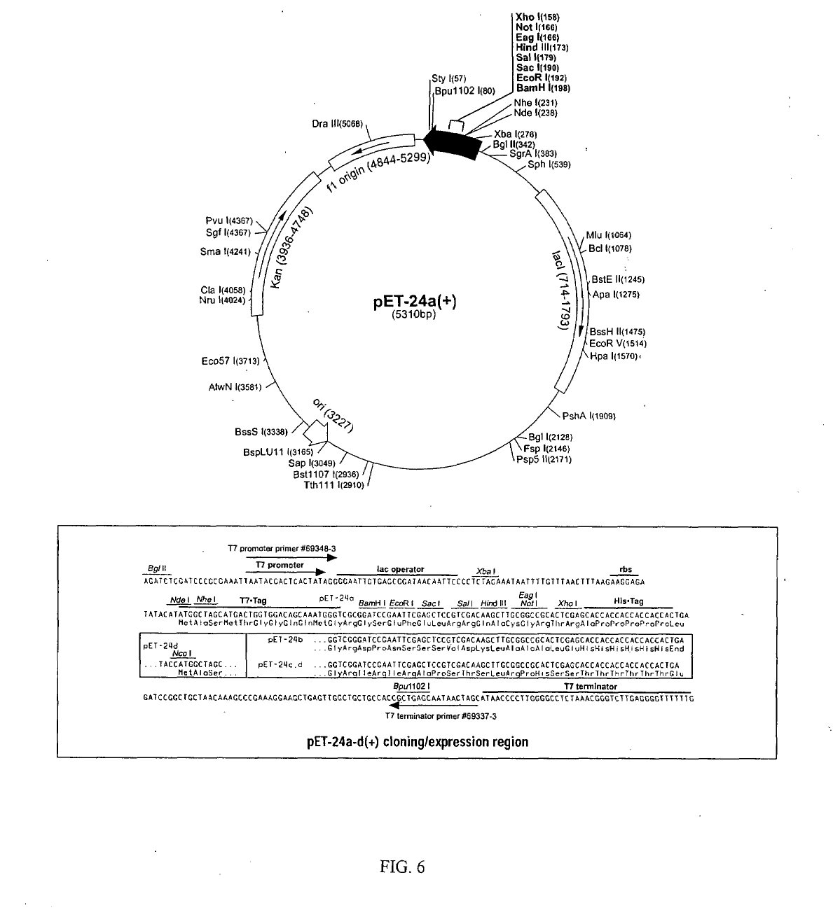

Vectors. Plasmid pCR2.1-TOPO vector; TOPO® Cloning vector (TA cloning kit, Invitrogen, Carlsbad, Calif.; herein incorporated by reference) was used to clone PCR products. Plasmid pET24a(+) (Novagen; herein incorporated by reference) was used as the expression vector for isolated TNAP genes. pET-24a-d(+) vectors carry an N-terminal T7•Tag® sequence plus an optional C-terminal His•Tag® sequence. These vectors differ from pET-21a-d(+) only by their selectable marker (kanamycin vs. ampicillin resistance). Note that the sequence is numbered by the pBR322 convention, so the T7 expression region is reversed on the vector map (see, FIG. 5). The f1 origin is oriented so that infection with helper phage will produce virions containing single-stranded DNA that corresponded to the coding strand.

I. Ligation Reactions: 1. For a standard reaction using DNA fragments with 2-4 base sticky ends, 50-100 ng (0.015-0.03 pmol) of pET vector was used with 0.2 pmol insert (e.g., 50 ng of a 500 bp fragment) in a volume of 20 μl. The following components were added in a 1.5-ml tube [available in the DNA Ligation Kit (Cat. No. 69838-3) and Clonables™ 2× Ligation Premix (Cat. No. 70573-3)]: 2 μl 10× Ligase Buffer (200 mM Tris-HCl, 100 mM MgCl₂, 250 μg/ml acetylated bovine serum albumin, pH 7.6); 2 μL 100 mM dithiothreitol; 1 μl mM ATP; 2 μl 50 ng/μl prepared pET vector; x μl Prepared heterologous gene insert (0.2 pmol); y ul Nuclease-free water to volume; 1 μl T4 DNA Ligase to 0.2-0.4 Weiss U/μl, diluted (with Ligase Dilution Buffer) (add ligase last); 20 μl Total volume; and 2. Gently mix by stirring with a pipet tip. Incubated at 16° C. for 2 h to overnight. A control reaction was performed in which the insert is omitted to check for non-recombinant background, see, protocol instructions).
II. Transformation: Competent cells, such as Novagen® NovaBlue and BL21(DE3), in standard kits were provided in 0.2-ml aliquots. Standard transformation reaction used 20 μl, so each tube contained enough cells for 10 transformations. Singles™ competent cells were provided in 50-μl aliquots, used “as is” for single 50-μl transformations.
III. Host Bacteria: Escherichia coli strains XL1-Blue MRF′ and XLOLR (Stratagene, La Jolla, Calif.; herein incorporated by reference) were used as host and excision plating strains for the T. neapolitana genomic library, respectively. Strain XL2-Blue (Stratagene; herein incorporated by reference) was used to select phoA point mutations. Strain BL21 (DE3) (Novagen, Madison, Wis.; herein incorporated by reference) was used to overexpress the TNAP fragments and genes. E. coli strains were grown in LB medium containing 50 μg/ml kanamycin, when necessary.

Examples I-II describe the isolation and sequencing of an N-terminal peptide from native TNAP whose sequence was used for designing nucleotide TNAP probes of the present invention. These TNAP nucleotide probes were used to screen a T. neapolitana genomic library for identifying and isolating TNAP sequences of the present invention as described in Examples III-V. Examples VI and VII describe the expression and purification of a recombinant thermostable TNAP with a high specific activity.

### Example I

### The Following Describes Materials and Methods Used for Isolated Native TNAP and for Trypsin Digestion of the Native Enzyme Followed by N-Terminal Sequencing

T. neapolitana bacteria. T. neapolitana used in the studies described herein was strain DSM 5068, originally obtained from Deutsche Sammlung von Mikroorganismen, Braunschweig (DSM), Germany. T. neapolitana bacteria were grown as described (Dong et al., (1997) Enzyme Microbial Technol. 21:335-340; herein incorporated by reference in its entirety). In brief, cells were grown at 80-85° C. in sealed culture bottles. Cells were harvested in the late exponential growth phase, chilled on ice, and pelleted by centrifugation (10,000×g, 40 min, 4° C.). The following methods describe isolated/purified hyperthermophilic TNAP by heat-treated T. neapolitana at 100° C. in the presence of Co(II) followed by ion-exchange and affinity chromatography.

Purification of T. neapolitana alkaline phosphatase (TNAP). Native TNAP enzyme was purified from 40-liter T. neapolitana cultures as described (Dong et al., (1997) Enzyme Microbial Technol. 21:335-340; herein incorporated by reference). Procedures were performed under room temperature and aerobic conditions unless otherwise stated. In brief: 1. Preparation of cell extract: Frozen cells (40 g wet mass) were suspended in 100 ml of 50 mM Tris-HCl buffer at pH 7.5 (“Buffer A”) containing 0.15% (w/v) Triton X-100 and stirred for 1 hour. After centrifugation at 16,300×g for 15 min., the pellet was extracted once more by repeating above procedure. The supernatants were pooled together and used as the crude enzyme preparation; 2. Heat treatment and (NH₄)₂SO₄ precipitation: 40 mM CoCl₂ was added to the cell extract after which the solution was heated for 20 min in a 100° C. water bath and then quickly cooled in a room temperature water bath. After centrifugation, the precipitate was discarded and 65% saturation (NH₄)₂SO₄ was added to the soluble fraction. The pellet obtained by ammonium sulfate precipitation was harvested by centrifugation and then suspended in 50 mM Tris-HCl buffer at pH 7.5 and dialyzed extensively against the same buffer at 4° C.; 3. Ion-exchange chromatography: The dialyzed enzyme (25 ml) from above treatment was applied to a DEAE-Sepharose column (2.6 cm×15 cm) equilibrated with Buffer A. The enzyme was eluted by applying a 0.0-0.4 M KCl linear gradient in Buffer A at a flow rate of 10 ml/tube/10 minutes. The AP activity was detected early in the elution; and 4. Affinity chromatography: The active fractions [as determined by enzyme assay as described in EXAMPLE V] from the ion-exchange column were pooled and loaded into a histidyldiazobenzylpropionic acid-Agarose column (1.0×6 cm) equilibrated with Buffer A. After washing, the nonspecific bound proteins were eluted with 1 M NaCl in Buffer A. Finally, the enzyme was eluted by pulse elution with 10 mM sodium phosphate in Buffer A.

In one example, a TNAP enzyme sample was purified approximately 2,880-fold with a 44% yield, see, for example, Dong, et al., supra. The following describes sequencing an N-terminal amino acid peptide of native TNAP for providing the nucleic acid hybridization probes of the present invention (for example, SEQ ID NO:16).

TNAP tryptic digestion and N-terminal sequencing. A TNAP enzyme sample was separated by sodium dodecyl sulfate-polyacrylamide gel electrophoresis (SDS-PAGE), by methods well-known in the art, (for example, Laemmli, (1970) Nature 227, 680-685; herein incorporated by reference), where an exemplary purified enzyme sample showed a single protein band of M(r) 45,000 daltons. A gel slice of this band was digested with trypsin as described in Wilm, et al. (1996) Nature 379:466-469; herein incorporated by reference. Tryptic peptides were separated on an HPLC C18 phase-coated 0.8×250 mm silica column (LC Packings, Switzerland), using methods well known in the art for collecting eluted fractions.

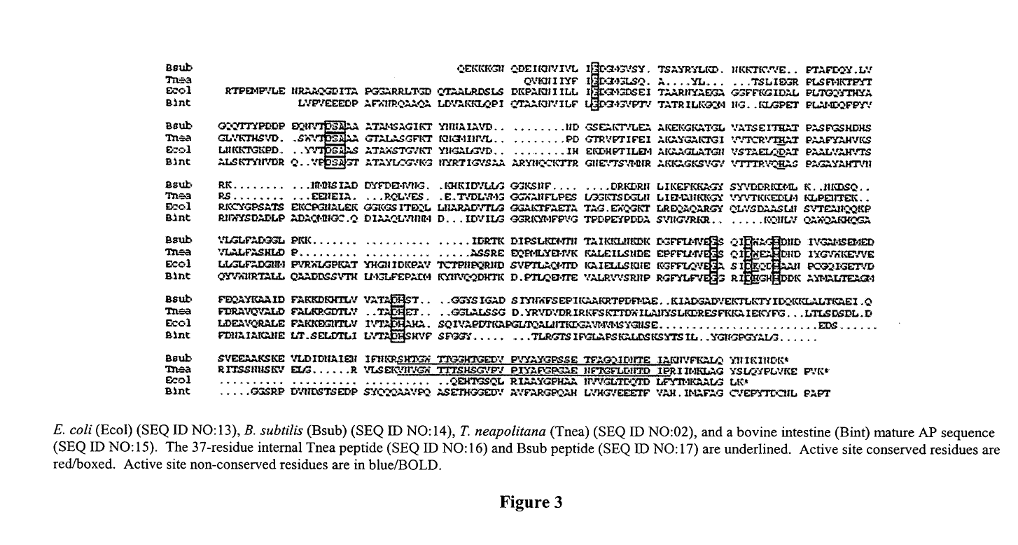

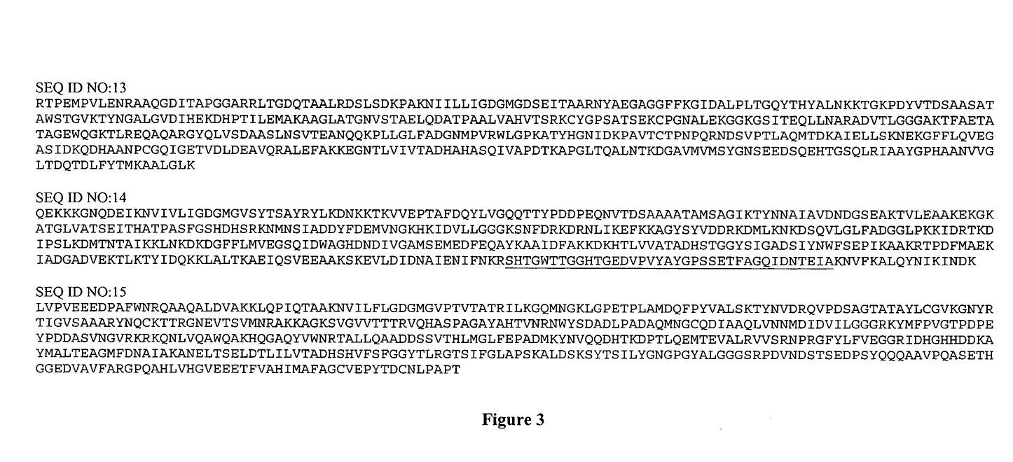

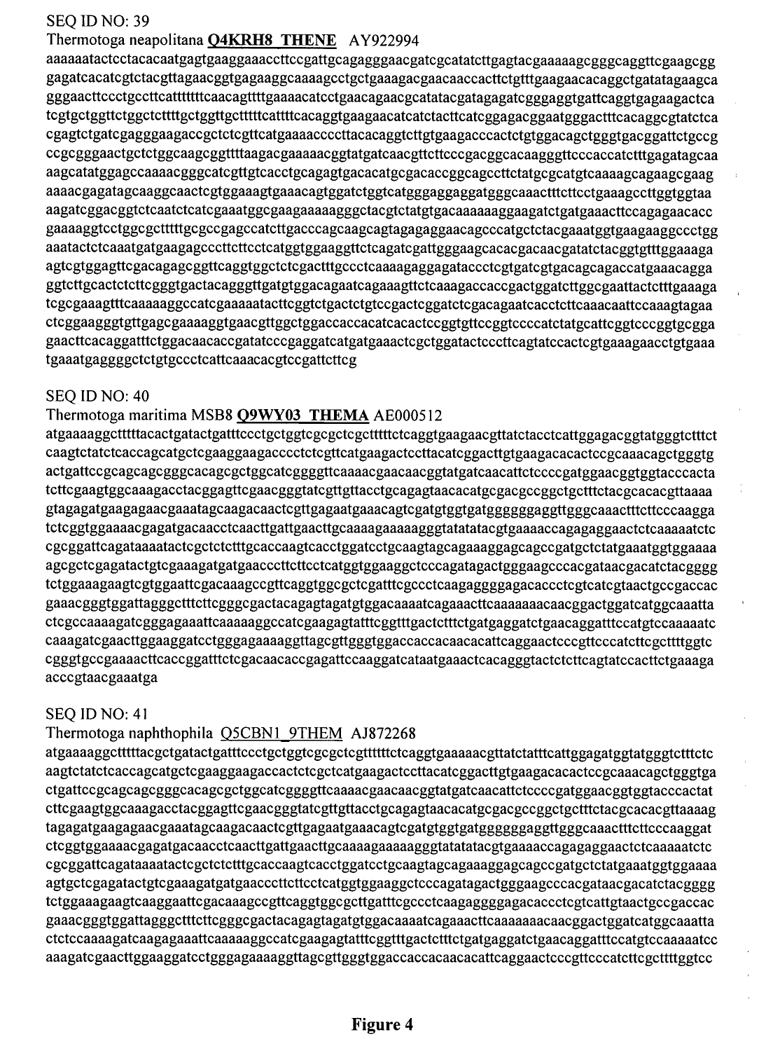

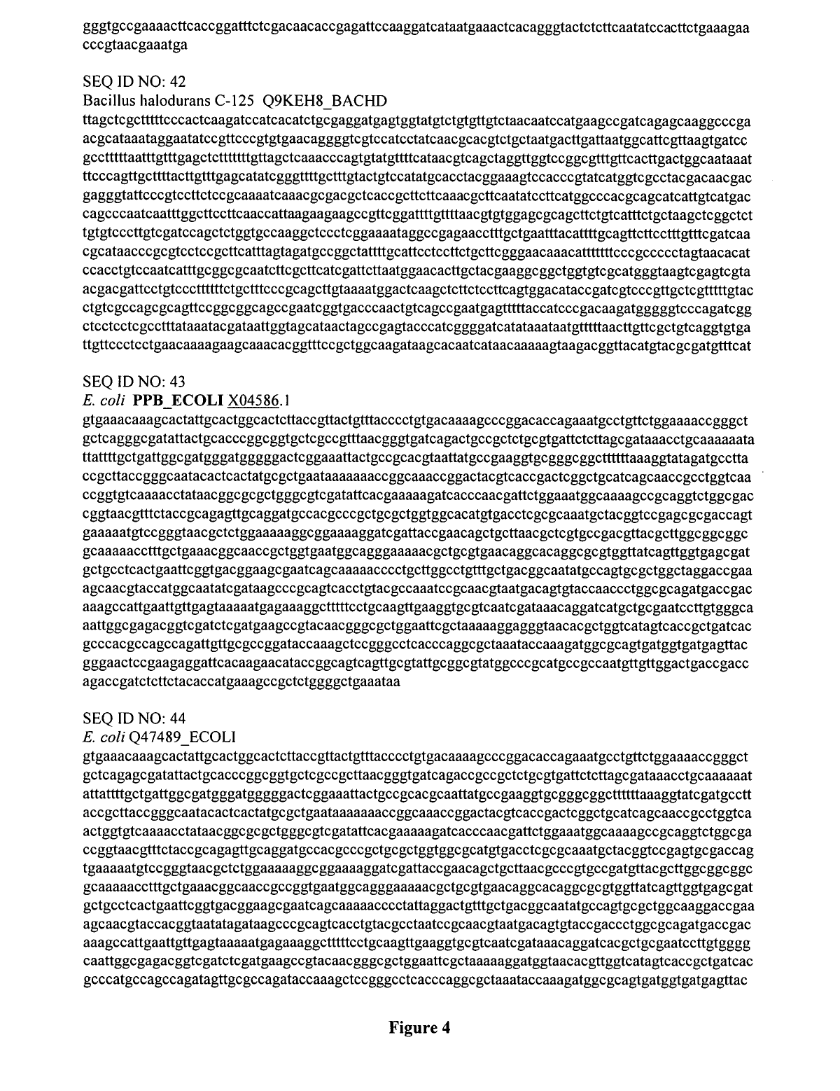

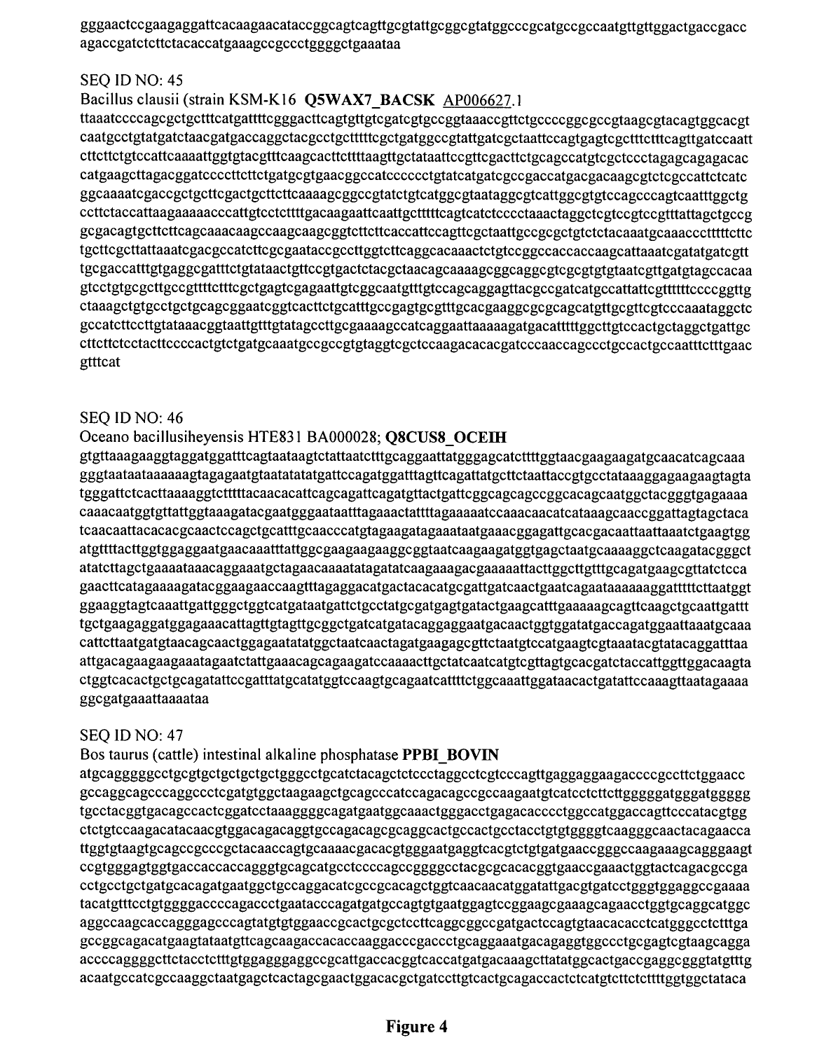

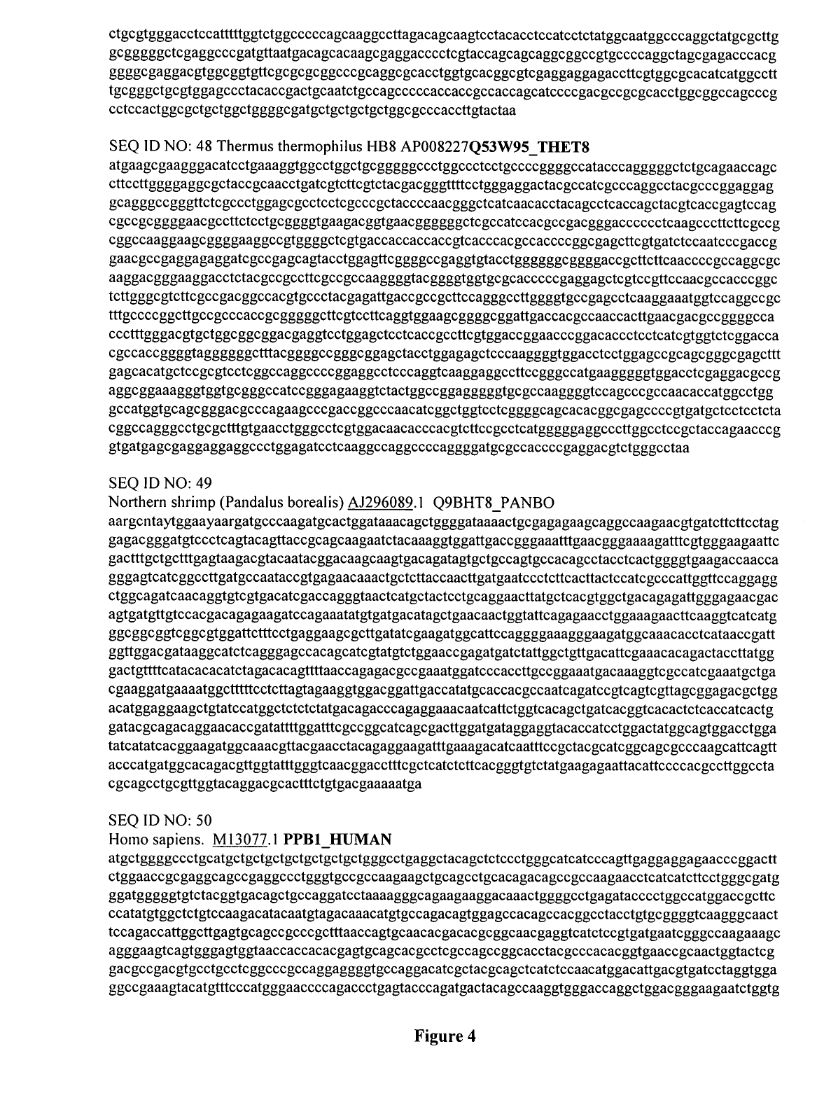

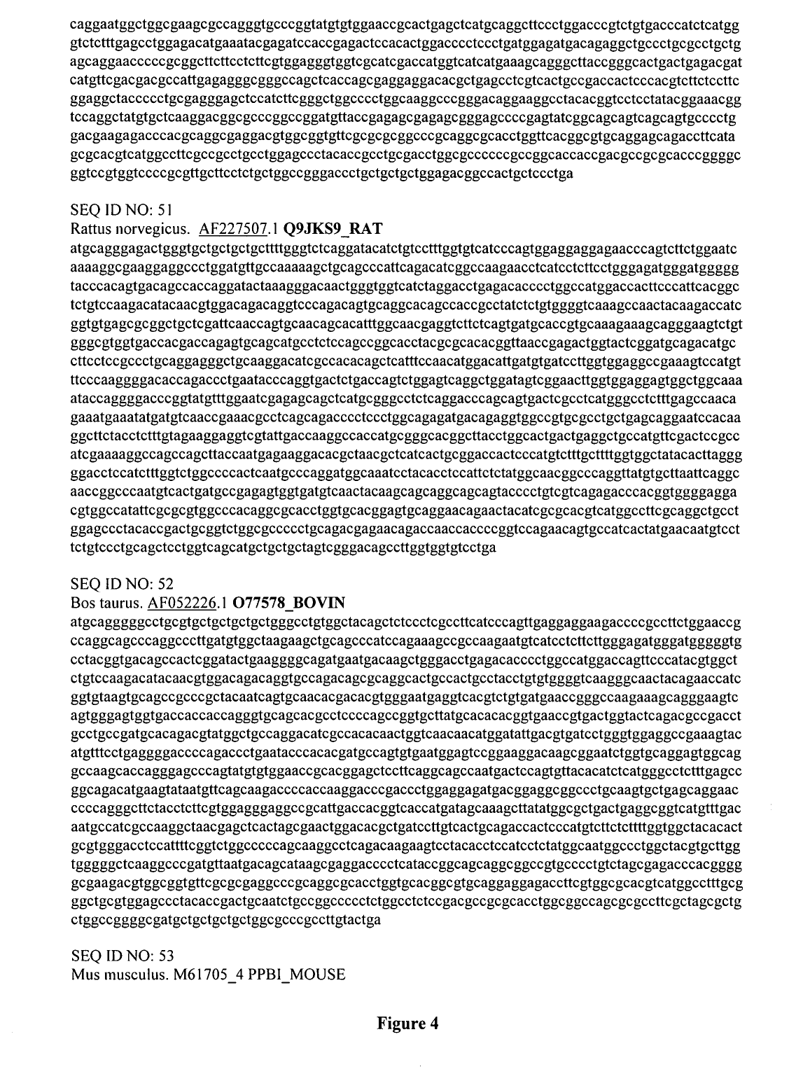

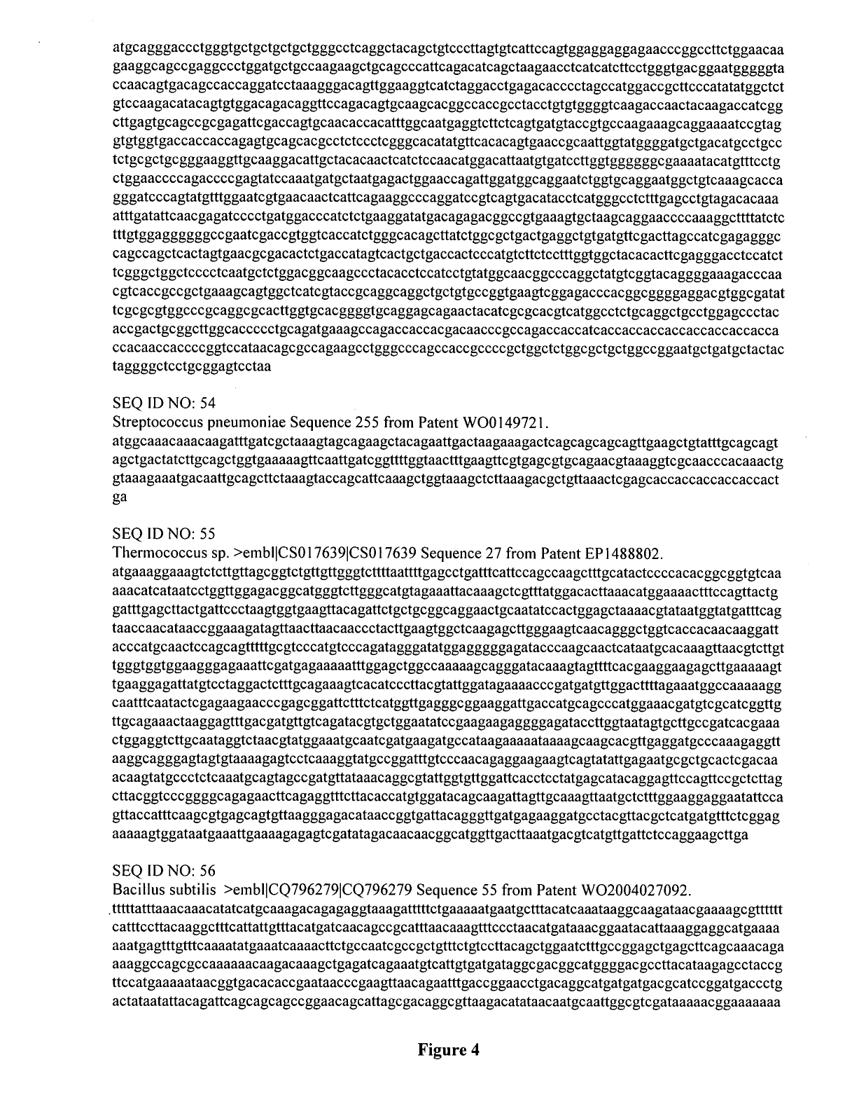

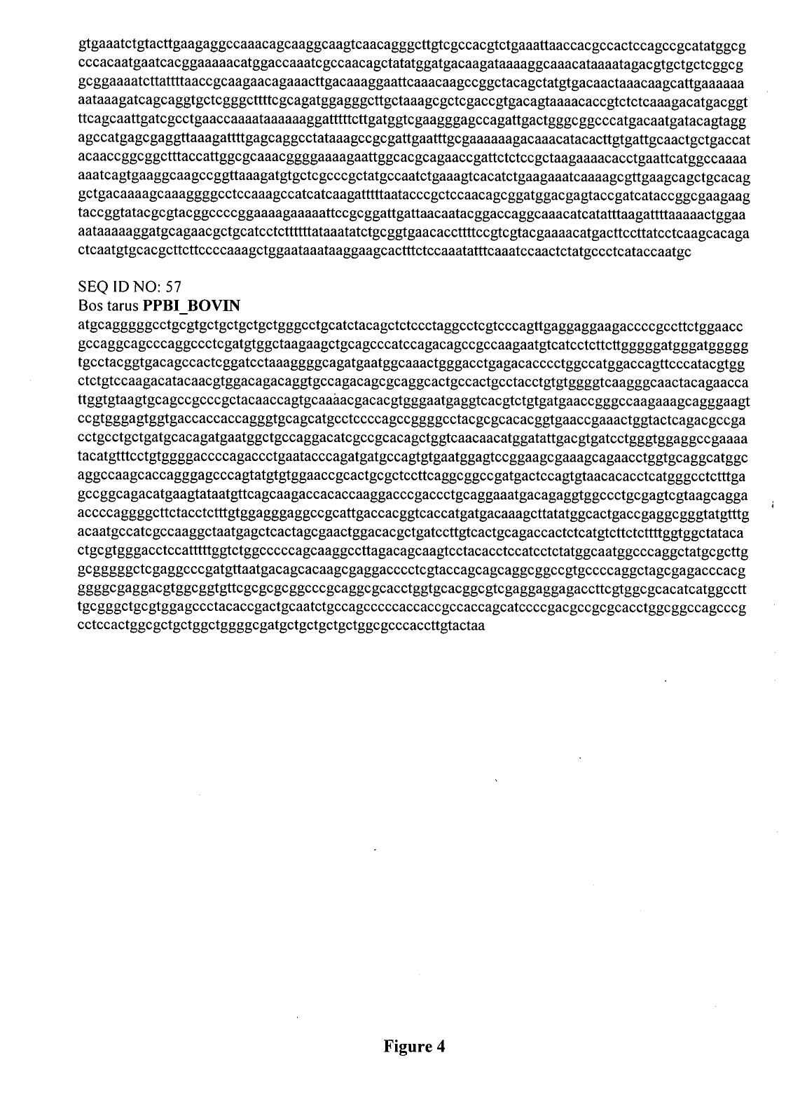

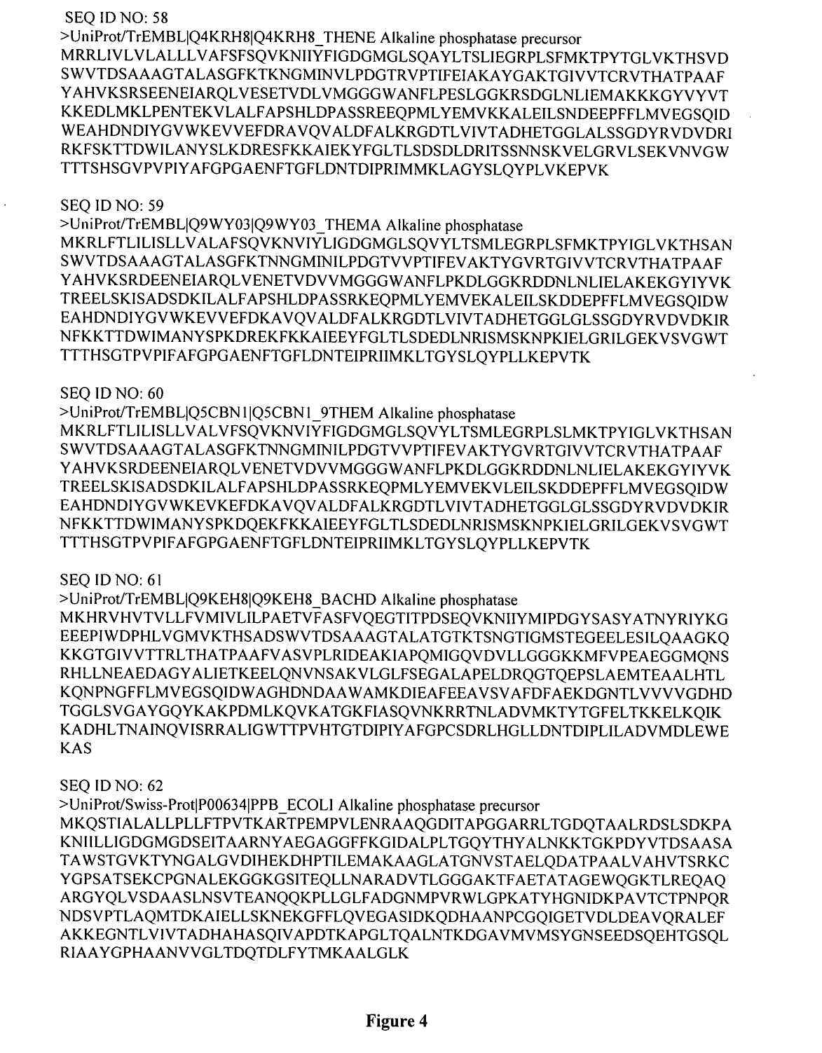

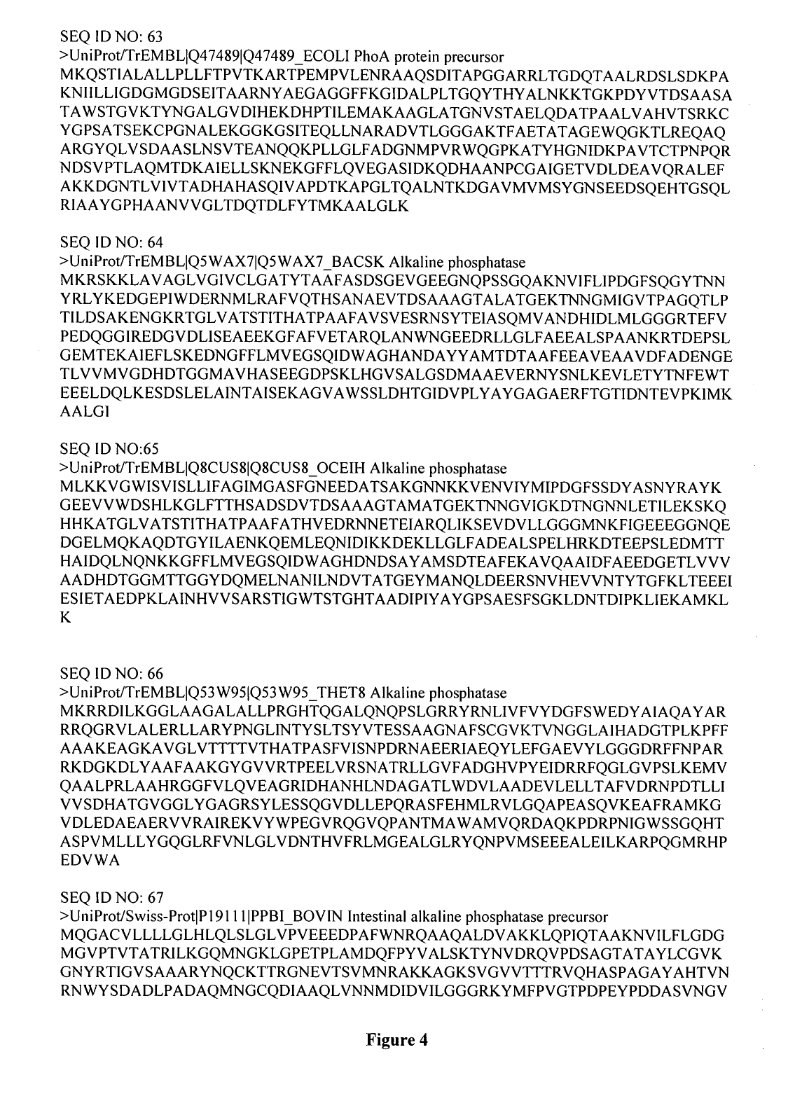

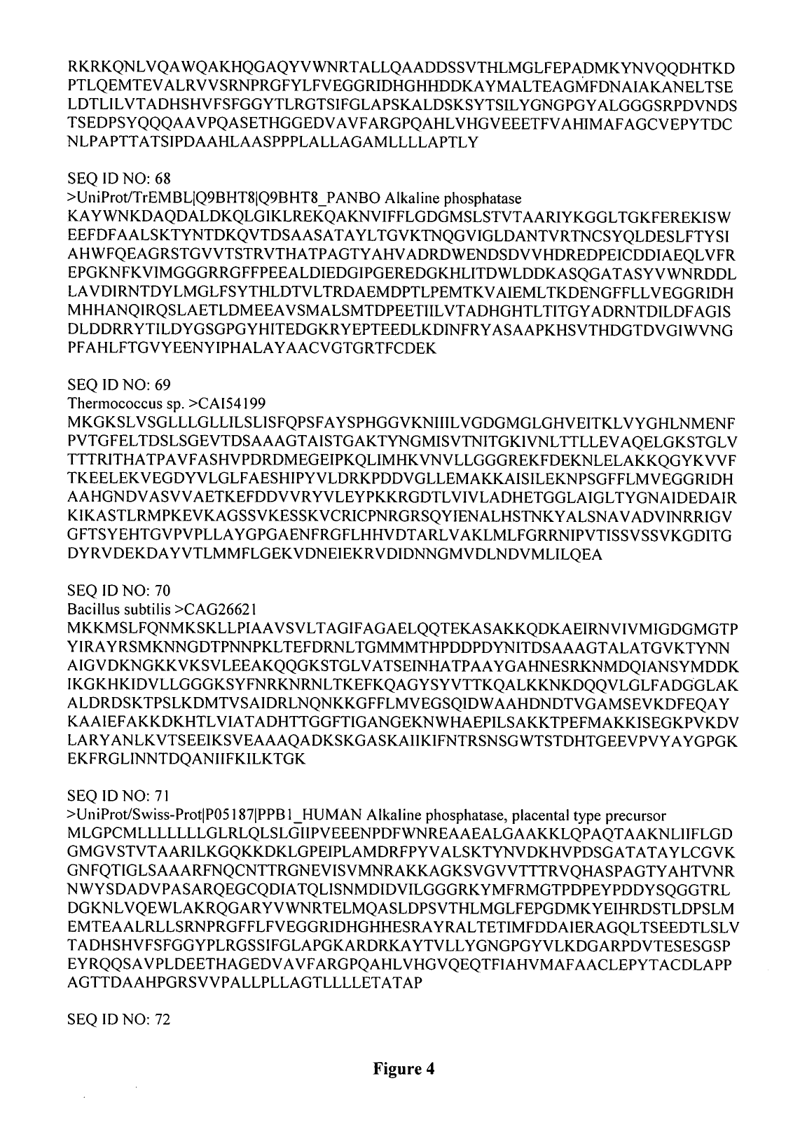

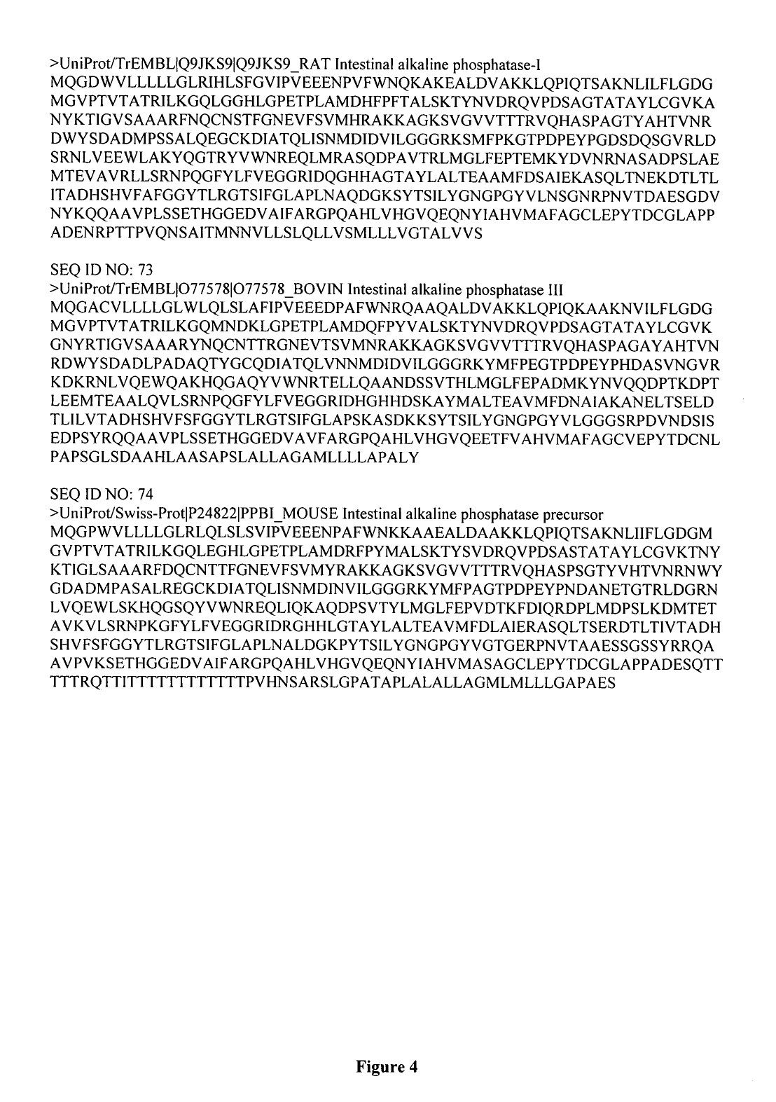

N-terminal sequence. Peptide N-terminal sequence analysis was performed on selected HPLC fractions at the Michigan State University Macromolecular Structure Facility by methods well-known in the art. An exemplary TNAP internal 37 amino acid-sequence VNVGWTTTSHSGVPVPIYAFGPGAENFTGFLDNTDIP (SEQ ID NO:16) was discovered using the methods described, supra. This sequence showed 62% similarity and 54% identity to Bacillus subtilis PhoA residues 411-447, SHTGWTTGGHTGEDVPVYAYGPSSETFAGQIDNTEIA, SEQ ID NO:17, FIG. 3. Further, this sequence showed a high percentage of identity to PhoA from Thermotoga spp. and lower identify levels to PhoA from other gram-negative bacteria such as Halobacterium, Pseudomonas aeruginosa, Flavobacterium, and from gram-positive, thermophilic Bacillus-related species, whose upper temperature limit for growth is 74° C. (optimally 60° C.) (e.g., Oceanobacillus iheyensis HTE831, isolated from deep-sea sediment, Geobacillus kaustophilus, isolated from hot spring and runoff, and Deinococcus geothermalis), for example, see, Table 1.

TABLE 1
TNAP-37 amino acid-sequence homologues.

| % identity | | Sequences similar to TNAP-37 amino acid-sequence | |
|---|---|---|---|
| to TNAP-37 | SEQ ID NO:xx | SEQ ID NO:16 | Organism |
| 100% | SEQ ID NO:16 | VNVGWTTTSHSGVPVPIYAFGPGAENFTGFLDNTDIP | T. neapolitana |
| 86% | SEQ ID NO:18 | VSVGWTTTTHSGTPVPIFAFGPGAENFTGFLDNTEIP | Thermotoga naphthophila |
| 86% | SEQ ID NO:19 | VSVGWTTTHSGTPVPIFAFGPGAENFTGFLDNTEIP | Thermotoga maritima |
| | | | MSB8 |
| 62% | SEQ ID NO:20 | IGWTSTGHTAADIPIYAYGPSAESFSGKLDNTDIP | Oceanobacillus iheyensis |
| | | | HTE831 |
| 59% | SEQ ID NO:21 | WTTTGHTAADVPLTAMGPGAERFTGIYENTRI | Geobacillus kaustophilus |
| 54% | SEQ ID NO:17 | SHTGWTTGGHTGEDVPVYAYGPSSETFAGQIDNTEIA | Bacillus subtilis |
| | | | PhoA residues |
| | | | 411-447 |
| 48% | SEQ ID NO:22 | HSADPVPLFAFGPGGNLFRNQIDQTEVT | Deinococcus geothermalis |
| | | | DSM 11300 |
| 43% | SEQ ID NO:23 | IAFTTDGHTGTDVPVFAHGPNAARFDAARDN | Halobacterium sp. |
| | | | NRC-1 |
| 41% | SEQ ID NO:24 | SSQEHTGTQLRIAAYGPQAANVTGLTDQTDL | Pseudomonas aeruginosa |
| | | | PAO1 |
| 41% | SEQ ID NO:25 | VAVYFITDQHSGELIPVFAAGKGADHFKGIYQNSAI | Flavobacterium sp. |
| | | | MED217 |
| 40% | SEQ ID NO:26 | FSTGGHSATLIPVFAYGPGSEEFIGIYENNEI | Antarctic bacterium |
| | | | TAB5 |

### Example II

### The Following Describes Materials and Methods Used for Production of TNAP Nucleic Acid Probes of the Present Invention

Synthetic degenerate oligonucleotides were designed for amplification of the 37-residue peptide-encoding DNA fragment, as described herein. In particular, primer 1, SEQ ID NO:30 and primer 2, SEQ ID NO:31 (encoding VNVGWT, SEQ ID NO:27 and ENFTGFL, SEQ ID NO:28, respectively, see amino acids in BOLD in Table 2) were used in standard PCR amplification reactions.

TABLE 2
Sequences of the 100-bp T. neapolitana
phoA internal fragment.

| | |
|---|---|
| GTGAACGTTGGCTGGACCACCACATCACACTCCGGTGTTCCGGTCCCCAT | (SEQ ID NO:11) |
| V N V G W T T T S H S G V P V P I | (SEQ ID NO:16) |
| CTATGCATTCGGTCCCGGTGCGGAGAACTTCACAGGATTTCTG | |
| Y A F G P G A E N F T G F L | |

The following oligonucleotides shown in Table 3 (synthesized at the Michigan State University Macromolecular Facility) were used for PCR reactions.

TABLE 3
Oligonucleotides used in this study.

| | | |
|---|---|---|
| SEQ ID NO:xx | Primer | Sequence |

| | | |
|---|---|---|
| SEQ ID NO:30 | 1 | 5′-GTNAAYGTNGGNTGGACNAC |
| SEQ ID NO:31 | 2 | 5′-CNARRAANCCNGTRAARTTYTCNG |
| SEQ ID NO:32 | S1 | 5′-GACCAGATCCACTGTTTCAC |
| SEQ ID NO:33 | S2 | 5′-CACGAGTTGCCTTGCTATC |
| SEQ ID NO:34 | 3 | 5′-TATCTCGAGTTTCACAGGTTCTTTCACG |
| | | AG |
| | | (underlined sequence creates a |
| | | XhoI site) |
| SEQ ID NO:35 | 4 | 5′-ATAGCTAGCCAGGTGAAGAACATCATCT |
| | | AC |
| | | (underlined sequence creates an |
| | | NheI site) |

### Example III

### The Following Describes the Materials and Methods Used for Identifying E. coli Carrying a Vector Comprising a TNAP Gene of the Present Invention

Genomic DNA preparation and library construction. T. neapolitana genomic (chromosomal) DNA was obtained from T. neapolitana 5068 (Belkin, et al (1986) Appl. Environ. Microbiol. 51:1180-1185) (DSM 5068) purified by the method of Goldberg and Ohman (Goldberg and Ohman (1984) J. Bacteriol. 158:1115-1121). Purified chromosomal DNA was partially digested with Sau3A generating genomic fragments. Two-to-twelve kb Sau3A fragments were isolated on a 10-40% sucrose gradient (Ausubel, et al., (ed.) (1993) Current protocols in molecular biology. Greene Publishing & Wiley-Interscience, New York) and cloned (ligated) into a BamHI/CIAP-treated ZAP Express vector using ZAP Express® (Catalog #239212 (ZAP Express® Predigested Vector Kit) and ZAP Express Predigested Gigapack II gold Cloning Kit ((Stratagene Catalog #239615); herein incorporated by reference) wherein the vector was predigested with BamH I, and dephosphorylated to prevent self-ligation. The library was screened by plaque hybridization using the probes described below, following the ZAP Express Cloning Kit instructions (Revision #053007, Stratagene).

TNAP probes. Hybridization TNAP nucleic acid probes for library screening were designed using purified native enzyme N-terminal amino acid sequence VNVGWTTTSHSGVPVPIYAFGPGAENFTGFL (SEQ ID NO:16), see, Table 2. Primers 1 and 2 (Table 3) were used to amplify a 100-bp fragment DNA TNAP probe that was cloned into vector pCR2.1 and sequenced, SEQ ID NO:11. Sequence of this 100-bp fragment confirmed that it encoded the expected AP internal sequence SEQ ID NO:16. Hybridization of a Southern blot of T. neapolitana genomic DNA with this DNA fragment as a probe confirmed that it would identify T. neapolitana genomic DNA. However, attempts at screening the T. neapolitana library using the 100-bp DNA fragment as a probe were unsuccessful (high hybridization background). Therefore, the inventors created a larger phoA-internal DNA fragment by cloning the phoA region upstream of the 100 bp-fragment using the LA PCR in vitro Cloning Kit (Takara Mirus Bio, WI). The resulting 900 bp-fragment contained the complete phoA 5′-fragment (SEQ ID NO:12). The 900-bp phoA fragment was then used to screen the T. neapolitana genomic DNA library. Forty-five positive clones were obtained after screening 3,000 recombinant phages. Two positive clones contained recombinant plasmids pTNAP1 and pTNAP2, with 3.5 and 4.6 kb inserts, respectively. Restriction enzyme analysis, using standard methods, confirmed the presence of the complete T. neapolitana phoA gene in both inserts.

### Example IV

The following describes the materials and methods used for cloning and sequencing TNAP of the present invention deposited in GenBank, under NCBI ACCESSION AY922994 (SEQ ID NO:01 and 02). A coding region was amplified by PCR and subcloned in the expression vector pET24a(+) NheI and XhoI sites (creating pTNAP4), Novagen•pET System Manual, 11th Edition, TB055 11th Edition 01/06; herein incorporated by reference.

Cloning of the T. neapolitana phoA gene. T. neapolitana (DSM 5068) genomic DNA, as described herein, was used as source DNA for the T. neapolitana phoA gene of the present invention.

Nucleotide sequence determination and sequence analysis. Sequences were determined on both strands using the ThermoSequenase radiolabeled terminator cycle sequencing kit (USB, Cleveland, Ohio). The cleavage site after the TNAP signal peptide was identified using the SignalP (v1.1) (Bendtsen, et al. (2004) J. Mol. Biol., 340:783-795, http://www.cbs.dtu.dk/services/SignalP; herein incorporated by reference in its entirety).

Nucleotide sequence of the T. neapolitana phoA gene. The T. neapolitana phoA gene was localized in the pTNAP1 3.5 kb insert by progressive sequencing, starting from the already known 100-bp internal sequence and sequencing toward both ends of the gene with phoA-specific primers. A unique 1,197-nt open reading frame (defined by an ATG and a stop codon) was detected (nucleotides 377 to 1573) in the six reading frames. Four valine GTG codons starting at nucleotides 275, 284, 299, and 344 were also considered as potential starting codons.

Similarity of the deduced peptidic sequence with E. coli AP started upstream of the first methionine (FIG. 1), suggesting that T. neapolitana phoA started with a GTG codon. The second GTG codon (starting at nucleotide 284) was preceded by a ribosome binding site (sequence GGAGGT, SEQ ID NO:36, complementary to the T. maritima 16S rRNA sequence 3′-CCUCCA-5′ SEQ ID NO:37, (Achenbach-Richter, et al, (1987) Syst. Appl. Microbiol. 9:34-39; herein incorporated by reference) (FIG. 1). Starting with valine, the 19 N-terminal amino acids matched the characteristics of a prokaryotic signal peptide (Nielsen et al., (1987) Protein Engin. 10:1-6; Watson (1984) Nucleic Acids Res. 12:5145-5264; all of which are herein incorporated by reference), including a cleavage site (FIG. 1) predicted using the SignalP WWW server, see, supra.

The mature TNAP, starting with sequence QVKNI, SEQ ID NO:38 of SEQ ID NO:08, contained 413 residues and had a calculated molecular weight of 45,668, in good agreement with the Mᵣ of 45,000 determined for the native enzyme by SDS-PAGE (Dong et al., (1997) Enzyme Microbial Technol. 21:335-340; herein incorporated by reference). A sequence identical to the 37-residue peptidic sequence determined by peptide sequencing was identified in the C-terminal part of the TNAP peptidic sequence (FIG. 1). Typical promoter and transcription termination signals were not identified upstream and downstream of the phoA gene, respectively.

Two types of methods were used to determine sequence homology to nucleic acid and amino acid sequences of the present inventions. First, WU-Blast2 comparisons were initiated through The European Bioinformatics Institute (EBI) website. Second, side by side alignments were made using coding regions obtained for amino acid sequences in Table 5.

The isolated T. neapolitana phoa nucleic acid (SEQ ID NO: 03) showed 76% and greater identity to hyperthermophilic bacterial sequences, and at least 60% identity to a thermophilic sequence, see, Table 4. The inventors also found high identity, 89%, to a short fragment (48 nucleic acids) of a nonthermophilic species Streptococcus pneumoniae, SEQ ID NO:54; however the present invention does not intend to include SEQ ID NO:54.

TABLE 4
Percent identity* of T. neapolitana phoA nucleic acid sequences encoding a
TNAP protein with a HIS tag; cloned into pET24a(+).

| % identity to | | | |
|---|---|---|---|
| TNAP na | SEQ ID NO: 03 | Organism | Accession No. |
| 100% | SEQ ID NO: 03 | Thermotoga | none |
| 1269/1269 | | neapolitana | |
| 98% | SEQ ID NO: 39 | Thermotoga | AY922994 |
| 1225/1242 | | neapolitana | Q4KRH8_THENE |
| 77% | SEQ ID NO: 40 | Thermotoga maritima | AE000512 |
| 968/1247 | | MSB8 | Q9WY03_THEMA |
| 76% | SEQ ID NO: 41 | Thermotoga | AJ872268 |
| 960/1247 | | naphthophila | Q5CBN1_9THEM |
| 89% | SEQ ID NO: 54 | Streptococcus | Sequence 255 from |
| 43/48 | | pneumoniae | Patent WO0149721 |
| 60.4% | SEQ ID NO: 48 | Thermus | AP008227 |
| | | thermophilus HB8 | Q53W95_THET8 |
| 59.7% | SEQ ID NO: 43 | E. coli | E. coli PPB_ECOLI |
| | | | X04586.1 |
| 58% | SEQ ID NO: 42 | Bacillus halodurans | BA000004 |
| 332/565 | | C-125 | BAB04593 |
| 58.5% | SEQ ID NO: 49 | Northern shrimp | AJ296089.1 |
| | | (Pandalus borealis) | Q9BHT8_PANBO |
| 58% | SEQ ID NO: 53 | Thermococcus sp. | CS017639 Sequence 27 from |
| 226/384 | | | Patent |
| | | | EP1488802 |
| 56% | SEQ ID NO: 46 | Oceanobacillus | BA000028; |
| 404/718 | | iheyensis HTE831 | Q8CUS8_OCEIH |
| 54% | SEQ ID NO: 56 | Bacillus subtilis | CQ796279 Sequence |
| 452/825 | | | 55 from Patent |
| | | | WO2004027092 |
| 54% | SEQ ID NO: 51 | Rattus norvegicus | Q9JKS9_RAT |
| 328/1269 | | (Rat) | |
| 53.0% | SEQ ID NO: 50 | Homo sapiens | PPB1_HUMAN |
| 421/1269 | | (Human) | |
| 53.0% | SEQ ID NO: 53 | Mus musculus | PPBI_MOUSE |
| 413/1269 | | | |
| 53.7% | SEQ ID NO: 52 | Bos taurus (Bovine) | O77578_BOVIN |
| 324/1269 | | | |
| 38% | SEQ ID NO: 45 | Bacillus clausii | Np**** |
| | | (strain | |
| | | KSM-K16 | |
| 28% | SEQ ID NO: 15 | Bos taurus | FIG. 3 |
| 25% | SEQ ID NO: 14 | B. subtilis | FIG. 3 |

*No filters, 100 alignments; (WU—Blast2-Washington University Basic Local Alignment Search Tool Version 2.0; The European Bioinformatics Institute (EBI)).
**na = information not available
***1269/1269 = number of identical na over total number of na in an aligned sequence.
Np**** = not provided.

### Example V

The following describes the materials and methods used for analyzing the catalytic activity of TNAP of the present invention.

Comparison with eubacterial and mammalian APs. A TNAP amino acid sequence (SEQ ID NO:02) of the present invention was 25 to 39% identical to mesophilic bacterial (i.e., B. clausii and E. coli) and eukaryotic (i.e., yeast and mammalian) enzymes. See, FIG. 3 and Table 5. Whereas TNAP showed a higher similarity 78-94% with published AP sequences from thermophilic and hyperthermophilic bacteria. See, Tables 5, below. Specifically, TNAP SEQ ID NO:02 was approximately 37%, 28.6%, and 25% identical to the B. subtilis (SEQ ID NO:14), E. coli, (SEQ ID NO:13), and bovine intestine (SEQ ID NO:15) enzymes, respectively. Moreover, the TNAP (SEQ ID NO:04), expressed by the cloned T. neapolitana phoA gene (SEQ ID NO:03) was 78% (326/414 aa) identical to a published Thermotoga maritima MSB8 AP (SEQ ID NO:58).

Structural determinants of TNAP catalytic activity. Alignment of these four AP sequences (FIG. 3) showed that the active site residues (red/boxed amino acids) and metal primary ligands (blue/BOLD amino acids) were conserved in the hyperthermophilic enzyme. Of particular interest are the differences in active site amino acids Asp(D)153 (underlined blue/BOLD) and Lys(K)328 (underlined blue/BOLD) in E. coli AP, the corresponding amino acid residues in TNAP, His(H)103 (underlined blue/BOLD) and Trp(W)328 (underlined blue/BOLD). Significantly more active than the E. coli enzyme (30 times), the mammalian homologs show His residues in these two positions. When introduced in E. coli AP, histidines at these two analogous sites increased the enzyme hydrolytic activity and shifted its pH profile. The altered residues at the identical sites, 153 and 328 in TNAP, as compared to H103 and W328 positions in mammalian (bovine) AP might partially explain why TNAP has a higher specific activity (30% more active than calf intestine AP [CIAP] (SEQ ID NO:15)).

Important functional differences were found between TNAP and its homologues. Specifically, certain amino acids in or near the catalytic sites associated with differences in enzyme activity, i.e. His-residues in positions 153 and/or 328 associated with a higher level of catalytic activity. One possible explanation for these differences comes from the analysis of TNAP catalytic site residues. Except for Asp153 and Lys328, the remainder of E. coli AP catalytic residues were conserved in TNAP. Residues Asp153 and Lys328 were instead His153 and Trp328, respectively, in TNAP. These two residues participate in the enzyme's interaction with Mg²⁺ and in phosphate binding (Murphy et al., (1994) Mol. Microbiol. 12:351-357; herein incorporated by reference). Mammalian APs invariably contain His-residues in these two positions. These same two residues have been shown to be the reason of mammalian APs's high specific activity and for the shift in their activity profile toward higher pHs (Murphy et al., (1995) J. Mol. Biol. 253:604 617; herein incorporated by reference).

When mammalian AP (Bint) is compared to E. coli AP, active site residues were conserved in E. coli and mammalian AP with the exception of residues corresponding to TNAP H103 (underlined blue/BOLD), T105 (underlined blue/BOLD), and W235. Mammalian and T. neapolitana enzymes have a His at position 103 while the E. coli enzyme has an Asp(D) (underlined blue/BOLD). Residue 105 is a Ser in mammalian enzymes and Thr(T) (underlined blue/BOLD) in E. coli and T. neapolitana APs. TNAP W235 (blue/BOLD underlined) is different from both mammalian and E. coli enzymes such that position 235 is His in mammalian APs and a Lys in the E. coli enzyme.

TABLE 5A
Percent identity* of AP amino acid sequences.

| % identity to | | | | |
|---|---|---|---|---|
| TNAP | Positives | SEQ ID NO: 02 | Organism | Accession No. |
| 100% | 100% | SEQ ID NO: 02 | Thermotoga | AY922994 |
| 433/433*** | 433/433 | | neapolitana | |
| 100% | 100% | SEQ ID NO: 57 | Thermotoga | Q4KRH8_THENE |
| 433/433 | 433/433 | | neapolitana | |
| 82% | 91% | SEQ ID NO: 58 | Thermotoga | Q9WY03_THEMA |
| 357/432 | 397/432 | | maritima MSB8 | |
| 81% | 91% | SEQ ID NO: 59 | Thermotoga | Q5CBN1_9THEM |
| 354/432 | 394/432 | | naphthophila | |
| 43% | 60% | SEQ ID NO: 60 | Bacillus halodurans | Q9KEH8_BACHD |
| 172/399 | 240/399 | | C-125 | |
| 42% | 50% | SEQ ID NO: 62 | E. coli | Q47489_ECOLI |
| 62/145 | 73/145 | | | |
| 41% | 61% | SEQ ID NO: 63 | Bacillus clausii | Q5WAX7_BACSK |
| 168/407 | 251/407 | | (strain KSM-K16) | |
| | | | (phoB) | |
| 40% | 62% | SEQ ID NO: 64 | Oceanobacillus | Q8CUS8_OCEIH |
| 163/404 | 252/404 | | iheyensis | |
| 37% | na** | SEQ ID NO: 14 | Bacillus subtilis, | FIG. 3 |
| 32% | 48% | SEQ ID NO: 65 | Thermus | Q53W95_THET8 |
| 119/363 | 176/363 | | thermophilus HB8 | |
| 30% | 45% | SEQ ID NO: 70 | Homo sapian | PPB1_HUMAN |
| 78/257 | 118/257 | | | |
| 28.6% | na | SEQ ID NO: 13 | E. coli (Ecol) | FIG. 3 |
| 27% | 46% | SEQ ID NO: 71 | Rattus norvegicus | Q9JKS9_RAT |
| 70/254 | 118/254 | | | |
| 26% | 45% | SEQ ID NO: 72 | Bos taurus | O77578_BOVIN |
| 69/257 | 118/257 | | | |
| 26% | 43% | SEQ ID NO: 73 | Mus musculus | PPBI_MOUSE |
| 94/352 | 153/352 | | | |
| 25% | na | SEQ ID NO: 68 | B. clausii | na |
| 25% | na | SEQ ID NO: 15 | Bos taurus | FIG. 3 |

*No filters, 350 alignments; (WU-Blast2-Washington University Basic Local Alignment Search Tool Version 2.0; The European Bioinformatics Institute (EBI)).
**na = information not available.
***433/433 = number of identical aa over total number of aa in an aligned sequence.

TABLE 5B
Percent identity* of expressed TNAP amino acid sequences (mature
TNAP without a signal sequence) compared to AP amino acid sequences.

| % identity to | | | | |
|---|---|---|---|---|
| TNAP aa | % Positive aa | SEQ ID NO: 04 | Organism | Accession No. |
| 100% | 100% | SEQ ID NO: 04 | Thermotoga | none |
| 422/422 aa | 422/422 | | neapolitana | |
| 94% | 94% | SEQ ID NO: 57 | Thermotoga | Q4KRH8_THENE |
| 392/415 | 392/415 | | neapolitana | |
| 78% | 78% | SEQ ID NO: 58 | Thermotoga maritima | Q9WY03_THEMA |
| 326/414 | 326/414 | | MSB8 | |
| 78% | 87% | SEQ ID NO: 59 | Thermotoga | Q5CBN1_9THEM |
| 323/414 | 361/414 | | naphthophila | |
| 48% | 62% | SEQ ID NO: 62 | E. coli | PPB_ECOLI |
| 40/83 | 52/83 | | | Q47489_ECOLI |
| 41% | 57% | SEQ ID NO: 60 | Bacillus halodurans | Q9KEH8_BACHD |
| 165/401 | 232/401 | | C-125 | |
| 38% | 58% | SEQ ID NO: 63 | Bacillus clausii (strain | Q5WAX7_BACSK |
| 158/408 | 240/408 | | KSM-K16 | |
| 38% | 60% | SEQ ID NO: 64 | Oceanobacillus | Q8CUS8_OCEIH |
| 155/404 | 243/404 | | iheyensis HTE831 | |
| 33% | 50% | SEQ ID NO: 66 | Bos taurus (Bovine) | PPBI_BOVIN |
| 47/142 | 72/142 | | | |
| 30% | 46% | SEQ ID NO: 67 | Northern shrimp | Q9BHT8_PANBO |
| 84/278 | 128/278 | | (Pandalus borealis) | |
| 29% | 44% | SEQ ID NO: 70 | Homo sapiens | PPB1_HUMAN |
| 76/257 | 115/257 | | (Human) | |
| 26% | 44% | SEQ ID NO: 71 | Rattus norvegicus | Q9JKS9_RAT |
| 66/253 | 113/253 | | (Rat) | |
| 26% | 44% | SEQ ID NO: 72 | Bos taurus (Bovine) | O77578_BOVIN |
| 67/257 | 114/257 | | | |
| 24% | 42% | SEQ ID NO: 73 | Mus musculus | PPBI_MOUSE |
| 72/292 | 125/292 | | | |

*No filters, 350 alignments; (WU-Blast2-Washington University Basic Local Alignment Search Tool Version 2.0; The European Bioinformatics Institute (EBI)).
**na = information not available
***433/433 = number of identical aa over total number of aa in an aligned sequence.

### Example VI

### The Following Describes the Materials and Methods Used for Expressing Recombinant TNAP

During the development of the present invention, the inventors found that a functional TNAP protein was not produced in pTNAP1, where TNAP was under control of its own Thermotoga promoter, and transformed into an AP-deficient Xph90a E. coli strain. Specifically, there was no significant increase in phosphatase activity detected in a cell lysate of the recombinant strain when compared to the strain without the expression plasmid as measured by the AP assay described in EXAMPLE VII.

TNAP expression in E. coli. First, pTNAP1 was transfected into the AP-deficient Xph90a nonlysogenic E. coli strain (Inouye, et al., (1981) J. Bacteriol. 146:668-675; herein incorporated by reference). However, no significant increase in phosphatase activity was detected in a cell lysate of the recombinant strain when compared to the same strain without pTNAP1. The inventors contemplated that the native TNAP promoter is not recognized/functional in E. coli. Next, the inventors constructed plasmid pTNAP3, in which T. neapolitana phoA gene expression was under control of the T7 promoter in a pET24a(+) vector (Novagen). Specifically, a 1.3 kb DNA fragment encoding the mature TNAP was amplified by PCR and cloned into the pET24a(+) vector, yielding plasmid pTNAP3. The amplified gene was verified as T. neapolitana phoA by DNA sequencing. The inventors found that by transforming E. coli strain BL21 (DE3) (Novagen, Madison, Wis.) where BL21 is lysogen for DE3, with the pTNAP3 construct, TNAP was expressed at high levels. In a preferred embodiment, the inventors express TNAP in a bacterium that would be a good host for heterologous protein expression, i.e. strain BL21(DE3) expressed fewer proteases than K12 E. coli derivatives.

pTNAP4. The T. neapolitana phoA gene (SEQ ID NO:01) was amplified using primers 4 and 5 containing Nhel an Xhol sites and subcloned in expression vector pET24a(+) (Novagen), generating plasmid pTNAP4. Plasmid pTNAP4 was transformed into the E. coli BL21(DE3) expression strain (Novagen). Transformed E. coli BL21(DE3) (pTNAP4) was grown at 37° C. in LB medium until density reached OD₆₀₀=0.7-0.9, then IPTG (1.2 mM) was added at 20-25° C. for 6 hours to induce protein expression. LB: Per liter: 10 g Tryptone; 5 g Yeast extract; 10 g NaCl; (pH was adjusted to 7.5 with 1N NaOH) then autoclaved to sterilize.

### Example VII

### The Following Describes the Materials and Methods Used for Purifying Recombinant TNAP

Purification of the recombinant TNAP. A recombinant enzyme was purified from E. coli BL21(DE3) containing pTNAP3. Briefly, the cells were harvested from a 1-liter overnight culture by centrifugation at 7,000 rpm for 15 min. The cell pellet was resuspended in 50 mM Tris-HCl (pH 7.0) containing 0.5 mg/ml lysozyme and incubated at room temperature for 20 min. Cells were then lysed in a French pressure cell. After centrifugation at 7,000 rpm for 15 min, the supernatant was used as the crude enzyme preparation. The recombinant protein was then purified by Ni-NTA affinity chromatography. Enzyme purity was judged by observing the bands seen after separation of samples by SDS-PAGE.

Purification of the native TNAP. T. neapolitana cells were suspended in Buffer A and stirred gently for 1 h. (some TNAP was found in the supernatant). Enzyme extraction efficiency was increased when 0.15% Triton® X-100 was added to the extraction buffer. After two extractions with Triton X-100, the majority of TNAP was recovered in the supernatant.

Protein was purified on Ni-NTA Agarose (Qiagen) using the Tris-HCl based buffer system (Invitrogen). Crude TNAP obtained from a cell extraction was highly thermostable. When this crude AP extract was heated at 100° C. for 40 min in the presence of 40 mM Co 2⁺, the residual activity was 97%, and the specific activity of the supernatant was increased 6.4 fold. The inventors found that Co²⁺ promoted strong affinity binding between the TNAP and the ligand in the subsequent affinity chromatography step. In the absence of Co²⁺, the TNAP did not bind to the histidyldiazobenzylpropionic acid-Agarose column, even at pH values between 6 and 10 and room temperature. In the presence of Co²⁺, most of the enzyme remained on the affinity column even after elution 1 M NaCl. The enzyme was totally eluted by 10 mM substrate such as pNPP or 10 mM of an inhibitor such as potassium phosphate. The affinity chromatography step resulted in greater purification of the TNAP. The native molecular weight of the protein was 87,000 estimated by gel filtration chromatography, indicating that the protein was homogenous dimer. The molecular weight was comparable to that of APs from other microorganisms such as E. coli and B. subtilis (see, McComb et al., supra).

Protein determination. Protein concentrations were determined using Bio-Rad solution (Sigma, U.S.A.) with bovine serum albumin as the standard protein. (Bradford, Anal. Biochem., 72:248-254 (1976)).

Alkaline phosphatase enzyme activity assay. TNAP activity was measured by following the release of p-nitrophenol from pNPP in 0.2 M Tris-HCl (pH 10.4) at 80° Celsius. Assays were done on at least two types of enzyme preparations, crude extract and purified enzyme. The reaction was initiated by adding 50 μl enzyme or 50 μl crude extract as part of dilution series into a cuvette containing 1 ml of 0.2 M Tris buffer (pH 9.9 at 60° C.) and 50 μl of 24 mM pNPP, preheated at 80° Celsius. The initial linear change in the absorbance at 410 nm was detected by a recording spectrophotometer (Cary 219, U.S.A.), thermostated at 80° Celsius. One enzyme activity unit represents the hydrolysis of 1 μmole of substrate per min under these standard assay conditions.

The optimal pH for the enzyme activity was measured using 0.2 M Tris-HCl buffer over a range of pH values and between 60° C. and 80° Celsius. pH values of buffers were measured at room temperature and corrected for pH change at high temperature using a Δpka/.ΔT° C. for Tris. (See, Perrin and B. Dempsey “Buffers for pH and Metal Ion Control,” Chapman & Hall, London, 157-163 (1974)). The temperature of maximal activity assays was determined using 0.2 M Tris-HCl at 60° C. and 80° Celsius. Because there was a small amount of non-enzymatic hydrolysis at a higher temperature, a control without enzyme also was performed.

When phosphate esters other than pNPP were used as substrates, AP activity was determined by measuring the amount of phosphate liberated during 10 min incubation at 80° Celsius. The incubation mixture contained 0.1 ml of 0.1 M substrate, 0.1 ml pure enzyme and 0.2 M Tris containing 5 mM CoCl₂ and 5 mM MgCl₂ in a total volume of 1 ml. Controls for non-enzymatic hydrolysis on each substrate were also performed. Samples were assayed for inorganic phosphate released by a modified method (Robyt and White, “Biochemical Techniques: Theory and Practice,” Brooks Cole, Monterey Calif. (1987)). As a comparison, the commercial CIP was also used to hydrolyze these phosphate esters. Reaction conditions for CIP were 0.1 M Tris-HCl buffer (pH 8.5) containing 50 mM MgCl₂ and 5 mM ZnCl₂ at 38° Celsius. Other reaction conditions were the same as above.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in biochemistry, chemistry, molecular biology, or related fields are intended to be within the scope of the following claims.

1. An expression vector comprising a nucleic acid at least 78% identical to SEQ ID NO:03 encoding an alkaline phosphatase polypeptide operably linked to an inducible promoter.

2. The expression vector of claim 1, wherein said alkaline phosphatase polypeptide comprises an amino acid sequence at least 79% identical to SEQ ID NO:04.

3. The expression vector of claim 1, wherein said nucleic acid comprises SEQ ID NO:11.

4. The expression vector of claim 1, wherein said nucleic acid is selected from the group consisting of SEQ ID NO:01, SEQ ID NO:03, SEQ ID NO:05, and SEQ ID NO:07.

5. The expression vector of claim 1, wherein said nucleic acid is derived from a hyperthermophilic bacterium.

6. The expression vector of claim 5, wherein said hyperthermophilic bacterium is a Thermotoga neapolitana.

7. The expression vector of claim 1, wherein said polypeptide comprises a C-terminal 6×His tag.

8. The expression vector of claim 1, further comprising a nucleic acid for increasing polypeptide production.

9. The expression vector of claim 8, wherein said nucleic acid encodes a protein for increasing extracellular export of said polypeptide.

10. The expression vector of claim 8, wherein said protein for increasing polypeptide production is a chaperone protein.

11. The expression vector of claim 8, wherein said polypeptide production increasing nucleic acid is selected from the group consisting of SEQ ID NOs: 75, 78, 79, 82, 84, and 86.

12. The expression vector of claim 1, wherein the inducible promoter is selected from the group consisting of isopropyl-β-D thiogalactopyranoside-inducible promoters.

13. A composition comprising a heterologous nucleic acid, wherein said nucleic acid is at least 78% identical to SEQ ID NO:03.

14. The composition of claim 13, wherein said nucleic acid encodes a polypeptide capable of alkaline phosphatase activity.

15. The composition of claim 14, wherein said alkaline phosphatase polypeptide demonstrates a specific activity of at least 1,500 U/mg at room temperature.

16. The composition of claim 14, wherein said alkaline phosphatase polypeptide demonstrates a specific activity of at least 3,000 U/mg at room temperature.

17. The composition of claim 14, wherein said alkaline phosphatase polypeptide demonstrates a specific activity of at least 10,000 U/mg at room temperature.

18. The composition of claim 13, wherein said nucleic acid derives from a hyperthermophilic bacterium.

19. The composition of claim 18, wherein said nucleic acid derives from a Thermotoga species.

20. The composition of claim 19, wherein said Thermotoga species is a T. neapolitana.

21. The composition of claim 13, further comprising an expression vector.

22. The composition of claim 13, wherein said nucleic acid is selected from the group consisting of SEQ ID NO:01, SEQ ID NO:03, SEQ ID NO:05, and SEQ ID NO:07.

23. The composition of claim 13, wherein said nucleic acid encodes a polypeptide at least 83% identical to SEQ ID NO:04.

24. The composition of claim 13, further comprising an E. coli host cell.

25. A method for providing commercial quantities of alkaline phosphatase, comprising,
a) providing,
i) a microorganism comprising an expression vector, wherein said expression vector comprises a nucleic acid at least 78% identical to SEQ ID NO:03, operably linked to an inducible promoter;
ii) an inducing agent for said inducible promoter;
ii) culture media for said microorganism; and
b) contacting said microorganism with an inducing agent for expressing a commercial quantity of alkaline phosphatase polypeptide in said culture media.

26. The method of claim 25, wherein said commercial quantity is at least 10 mg of purified enzyme per liter of culture media.

27. The method of claim 25, wherein said commercial quantity is at least 15 mg of purified enzyme per liter of culture media.

28. The method of claim 25, wherein said nucleic acid encodes a polypeptide at least 79% identical to SEQ ID NO:4.

29. The method of claim 28, wherein said polypeptide comprises a C-terminal 6×His tag.

30. The method of claim 25, wherein the inducible promoter from the group consisting of isopropyl-β-D thiogalactopyranoside-inducible promoters.

31. The method of claim 25, wherein the inducing agent is isopropyl-β-D thiogalactopyranoside.

32. The method of claim 25, wherein the microorganism is an E. coli.

33. The method of claim 25, wherein said expression vector further comprises a nucleic acid for enhancing polypeptide secretion into said culture media.

34. The method of claim 25, wherein said nucleic acid for enhancing polypeptide secretion is selected from the group consisting of a chaperone polypeptide, a chaperonin polypeptide, a polypeptide-export polypeptide, a polypeptide translocase polypeptide, a Sec export polypeptide, a Sec-independent translocase polypeptide, and a twin-arginine leader-binding polypeptide.

35. The method of claim 34, wherein said secretion enhancing nucleic acid is selected from the group consisting of SEQ ID NOs:75, 78, 79, 82, 84, and 86.
